SEQUENCE LISTING (64 sequences)

SEQ ID NO: 1 - protein, 7 aa, Artificial Sequence
  Description of Artificial Sequence Synthetic peptide
YEYLDYD

SEQ ID NO: 2 - protein, 20 aa, Homo sapiens
VRPEHPAETEYDSLYPEDDL

SEQ ID NO: 3 - protein, 6 aa, Artificial Sequence
  Description of Artificial Sequence Synthetic consensus amino acid sequence
XXXPXX

SEQ ID NO: 4 - protein, 6 aa, Artificial Sequence
  Description of Artificial Sequence Synthetic peptide
MRAPVI

SEQ ID NO: 5 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARLRRPSIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 6 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARMRRPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 7 - protein, 17 aa, Artificial Sequence
  Description of Artificial Sequence Synthetic peptide
QATEYEYLDYDFLPETE

SEQ ID NO: 8 - protein, 15 aa, Artificial Sequence
  Description of Artificial Sequence Synthetic linker peptide
GGGGSGGGGSGGGGS

SEQ ID NO: 9 - protein, 6 aa, Artificial Sequence
  Description of Artificial Sequence Synthetic peptide
LRRPSI

SEQ ID NO: 10 - protein, 6 aa, Artificial Sequence
  Description of Artificial Sequence Synthetic peptide
MRRPVI

SEQ ID NO: 11 - protein, 6 aa, Artificial Sequence
  Description of Artificial Sequence Synthetic peptide
LVMPVM

SEQ ID NO: 12 - protein, 6 aa, Artificial Sequence
  Description of Artificial Sequence Synthetic peptide
LWYPFG

SEQ ID NO: 13 - protein, 6 aa, Artificial Sequence
  Description of Artificial Sequence Synthetic peptide
LRVPFL

SEQ ID NO: 14 - protein, 6 aa, Artificial Sequence
  Description of Artificial Sequence Synthetic peptide
LRSPFG

SEQ ID NO: 15 - protein, 6 aa, Artificial Sequence
  Description of Artificial Sequence Synthetic peptide
LSPPIF

SEQ ID NO: 16 - protein, 6 aa, Artificial Sequence
  Description of Artificial Sequence Synthetic peptide
LLPPYG

SEQ ID NO: 17 - protein, 16 aa, Artificial Sequence
  Description of Artificial Sequence Synthetic peptide
GINWNGGSTGYADSVK

SEQ ID NO: 18 - protein, 8 aa, Artificial Sequence
  Description of Artificial Sequence Synthetic peptide
LNPKVKHM

SEQ ID NO: 19 - protein, 5 aa, Artificial Sequence
  Description of Artificial Sequence Synthetic linker peptide
GGGGS

SEQ ID NO: 20 - DNA, 63 bp, Artificial Sequence
  Description of Artificial Sequence Synthetic oligonucleotide
ccgggacagcagtggtaaccatgtggtattcggcggagggaccaagctgaccgtcctaggtgg

SEQ ID NO: 21 - DNA, 63 bp, Artificial Sequence
  Description of Artificial Sequence Synthetic oligonucleotide
ggccccacctaggacggtcagcttggtccctccgccgaataccacatggttaccactgctgtc

SEQ ID NO: 22 - DNA, 21 bp, Artificial Sequence
  Description of Artificial Sequence Synthetic oligonucleotide
tggggccaaggtaccctggtc

SEQ ID NO: 23 - DNA, 20 bp, Artificial Sequence
  Description of Artificial Sequence Synthetic oligonucleotide
cactgctgtcccgggagtta

SEQ ID NO: 24 - DNA, 68 bp, Artificial Sequence
  Description of Artificial Sequence Synthetic oligonucleotide
agctttcggccgtgtattactgtgcaagannknnknnknnknnknnktggggccaaggtaccctggtc

SEQ ID NO: 25 - DNA, 32 bp, Artificial Sequence
  Description of Artificial Sequence Synthetic oligonucleotide
ttcagctttcggccgtgtattactgtgcaaga

SEQ ID NO: 26 - protein, 17 aa, Artificial Sequence
  Description of Artificial Sequence Synthetic peptide
QATEYEYLDYDFLPETE

SEQ ID NO: 27 - protein, 6 aa, Artificial Sequence
  Description of Artificial Sequence Synthetic peptide
LRDPIM

SEQ ID NO: 28 - protein, 6 aa, Artificial Sequence
  Description of Artificial Sequence Synthetic peptide
LRPPFL

SEQ ID NO: 29 - protein, 6 aa, Artificial Sequence
  Description of Artificial Sequence Synthetic peptide
LTYPHL

SEQ ID NO: 30 - protein, 6 aa, Artificial Sequence
  Description of Artificial Sequence Synthetic peptide
LKWPHL

SEQ ID NO: 31 - protein, 6 aa, Artificial Sequence
  Description of Artificial Sequence Synthetic peptide
LRYPFF

SEQ ID NO: 32 - protein, 6 aa, Artificial Sequence
  Description of Artificial Sequence Synthetic peptide
LRSPVL

SEQ ID NO: 33 - protein, 6 aa, Artificial Sequence
  Description of Artificial Sequence Synthetic peptide
VRHPIM

SEQ ID NO: 34 - protein, 6 aa, Artificial Sequence
  Description of Artificial Sequence Synthetic peptide
LRHPVA

SEQ ID NO: 35 - protein, 6 aa, Artificial Sequence
  Description of Artificial Sequence Synthetic peptide
LRPPVM

SEQ ID NO: 36 - protein, 6 aa, Artificial Sequence
  Description of Artificial Sequence Synthetic peptide
LRFPIA

SEQ ID NO: 37 - protein, 6 aa, Artificial Sequence
  Description of Artificial Sequence Synthetic peptide
LRSPVL

SEQ ID NO: 38 - protein, 6 aa, Artificial Sequence
  Description of Artificial Sequence Synthetic peptide
LRSPPI

SEQ ID NO: 39 - protein, 6 aa, Artificial Sequence
  Description of Artificial Sequence Synthetic peptide
LAQPFG

SEQ ID NO: 40 - protein, 6 aa, Artificial Sequence
  Description of Artificial Sequence Synthetic peptide
MRSPYK

SEQ ID NO: 41 - protein, 6 aa, Artificial Sequence
  Description of Artificial Sequence Synthetic peptide
FSSPHA

SEQ ID NO: 42 - protein, 6 aa, Artificial Sequence
  Description of Artificial Sequence Synthetic peptide
LPSFRV

SEQ ID NO: 43 - protein, 18 aa, Artificial Sequence
  Description of Artificial Sequence Synthetic peptide
GDEGDTDLYDYYPEEDTE

SEQ ID NO: 44 - protein, 18 aa, Artificial Sequence
  Description of Artificial Sequence Synthetic peptide
GDEGDTDLYDYYPEEDTE

SEQ ID NO: 45 - protein, 6 aa, Artificial Sequence
  Description of Artificial Sequence Synthetic peptide
MAAPVI

SEQ ID NO: 46 - protein, 6 aa, Artificial Sequence
  Description of Artificial Sequence Synthetic peptide
MRAPRI

SEQ ID NO: 47 - protein, 6 aa, Artificial Sequence
  Description of Artificial Sequence Synthetic peptide
IPMARV

SEQ ID NO: 48 - protein, 17 aa, Artificial Sequence
  Description of Artificial Sequence Synthetic peptide
QATEYEYLDYDFLPETE

SEQ ID NO: 49 - protein, 17 aa, Artificial Sequence
  Description of Artificial Sequence Synthetic peptide
QATEYEYLDYDFLPETE

SEQ ID NO: 50 - protein, 18 aa, Artificial Sequence
  Description of Artificial Sequence Synthetic peptide
GDEGDTDLYDYYPEEDTE

SEQ ID NO: 51 - protein, 18 aa, Artificial Sequence
  Description of Artificial Sequence Synthetic peptide
MDYQVSSPIYDINYYTSE

SEQ ID NO: 52 - protein, 18 aa, Artificial Sequence
  Description of Artificial Sequence Synthetic peptide
MDYQVSSPIYDINYYTSE

SEQ ID NO: 53 - protein, 18 aa, Artificial Sequence
  Description of Artificial Sequence Synthetic peptide
MDYQVSSPIYDINYYTSE

SEQ ID NO: 54 - protein, 18 aa, Artificial Sequence
  Description of Artificial Sequence Synthetic peptide
MDYQVSSPIYDINYYTSE

SEQ ID NO: 55 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARLRSPFGWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 56 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARLRVPFLWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 57 - protein, 18 aa, Artificial Sequence
  Description of Artificial Sequence Synthetic peptide
GDEGDTDLYDYYPEEDTE

SEQ ID NO: 58 - protein, 18 aa, Artificial Sequence
  Description of Artificial Sequence Synthetic peptide
GDEGDTDLYDYYPEEDTE

SEQ ID NO: 59 - protein, 17 aa, Artificial Sequence
  Description of Artificial Sequence Synthetic peptide
QATEYEYLDYDFLPETE

SEQ ID NO: 60 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARLRPPFLWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 61 - protein, 280 aa, Artificial Sequence
  Description of Artificial Sequence Synthetic consensus antibody
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARLRYPFFWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 62 - protein, 18 aa, Artificial Sequence
  Description of Artificial Sequence Synthetic peptide
MDYQVSSPIYDINYYTSE

SEQ ID NO: 63 - protein, 6 aa, Artificial Sequence
  Description of Artificial Sequence Synthetic 6xHis tag
HHHHHH

SEQ ID NO: 64 - protein, 6 aa, Artificial Sequence
  Description of Artificial Sequence Synthetic tag sequence
HTTPHH

(19) United States
(12) Patent Application Publication
(10) Pub. No.: US 2008/0274100 A1
(43) Pub. Date: Nov. 6, 2008

(54) ANTIBODIES AND USES THEREOF

(76) Inventors: Rachel Ben-Levy, Relem (IL); Yael Nisgav, Petach-Tikva (IL); Yariv Kanfi, Petach Tikva (IL)

Correspondence Address: KENYON & KENYON LLP, ONE BROADWAY, NEW YORK, NY, 10004, US

(21) Appl. No.: 11/969,005

(22) Filed: Jan. 3, 2008

Related U.S. Application Data
(63) Continuation of application No. 11/166,740, filed on Jun. 27, 2005, now abandoned.
Application No. 11/166,740 is a continuation-in-part of application No. 10/880,922, filed on Jun. 30, 2004, now abandoned.
(60) Provisional application No. 60/484,061, filed on Jun. 30, 2003.

(51) Int. Cl. A61K 39/395; C07K 16/00; C07K 16/36; A61P 31/18

(52) U.S. Cl. 424/130.1; 530/387.1; 530/389.3

(57) ABSTRACT

The present invention provides antibodies or fragments thereof that bind to co-receptor CCR5 or CXCR4.

### RELATED APPLICATIONS

This application is a continuation-in-part of U.S. application Ser. No. 10/880,922, which was filed on Jun. 30, 2004.

### FIELD OF THE INVENTION

The present invention relates to antibodies that bind to particular epitopes that are present on cells, such as cancer cells, metastatic cells, leukemia cells, leukocytes, and platelets, and that are important in such diverse physiological phenomena as cell rolling, metastasis, inflammation, and auto-immune diseases. More particularly, the antibodies may have anti-cancer activity, anti-metastatic activity, anti-leukemia activity, anti-viral activity, anti-infection activity, and/or activity against other diseases, such as inflammatory diseases, autoimmune diseases, HIV infection, cardiovascular diseases such as myocardial infarction, retinopathic diseases, and diseases caused by sulfated tyrosine-dependent protein-protein interactions. In addition, the antibodies of the present invention may be used as a targeting agent to direct a therapeutic to a specific cell or site within the body.

### BACKGROUND OF THE INVENTION

### Antibodies, Phage Display, and Tissue Targeting

Tissue-selective targeting of therapeutic agents is an emerging discipline in the pharmaceutical industry. New cancer treatments based on targeting have been designed to increase the specificity and potency of the treatment while reducing toxicity, thereby enhancing overall efficacy. Mouse monoclonal antibodies (MAbs) to tumor-associated antigens have been employed in an attempt to target toxin, radionucleotide, and chemotherapeutic conjugates to tumors. In addition, differentiation antigens, such as CD19, CD20, CD22, and CD25, have been exploited as cancer specific targets in treating hematopoietic malignancies.

Although extensively studied, this approach has several limitations. One limitation is the difficulty of isolating appropriate MAbs that display selective binding. A second limitation is the need for high antibody immunogenicity as a prerequisite for successful antibody isolation. A third limitation is that the final product has non-human sequences, which induce immune responses; e.g., when a mouse MAb is given to a human, a human anti-mouse antibody (HAMA) response will be generated. The HAMA response often results in a shorter serum half-life and prevents repetitive treatments, thus diminishing the therapeutic value of the antibody. This latter limitation has stimulated interest both in engineering chimeric or humanized monoclonal antibodies of murine origin and in discovering human antibodies. Another limitation of this approach is that it enables the isolation of only a single antibody species directed against only known and purified antigens. Moreover, this method is not selective insofar as it allows for the isolation of antibodies against cell surface markers that are present on normal, as well as malignant, cells.

There are many factors that influence the therapeutic efficacy of MAbs for treating cancer. These factors include the specificity and level of antigen expression on tumor cells, antigenic heterogeneity, and accessibility of the tumor mass. Leukemias and lymphomas have been generally more responsive to treatment with antibodies than solid tumors, such as carcinomas. MAbs rapidly bind to leukemia and lymphoma cells in the bloodstream and easily penetrate to malignant cells in lymphatic tissue, thus making lymphoid tumors excellent candidates for MAb-based therapy. An ideal system entails identifying a MAb that recognizes a marker on the cell surface of stem cells that are producing malignant progeny cells.

Phage libraries are used to select random single chain variant fragments (scFvs) that bind to isolated, pre-determined target proteins such as antibodies, hormones, and receptors. In addition, the use of antibody display libraries in general, and phage scFv libraries in particular, facilitates an alternative means of discovering unique molecules for targeting specific, yet unrecognized and undetermined, cell surface moieties.

Leukemia, lymphoma, and myeloma are cancers that originate in the bone marrow and lymphatic tissues and are involved in uncontrolled growth of cells. Acute lymphoblastic leukemia (ALL) is a heterogeneous disease that is defined by specific clinical and immunological characteristics. Like other forms of ALL, the definitive cause of most cases of B cell ALL (B-ALL) is not known; although, in many cases, the disease results from acquired genetic alterations in the DNA of a single cell, causing abnormalities and continuous multiplication. Prognosis for patients afflicted with B-ALL is significantly worse than for patients with other leukemias, both in children and in adults. Chronic lymphocytic leukemia (CLL) is a slowly progressing form of leukemia, characterized by an increased number of lymphocytes. Acute myelogenous leukemia (AML) is a heterogeneous group of neoplasms with a progenitor cell that, under normal conditions, gives rise to terminally differentiated cells of the myeloid series (erythrocytes, granulocytes, monocytes, and platelets). As in other forms of neoplasia, AML is associated with acquired genetic alterations that result in replacement of normally differentiated myeloid cells with relatively undifferentiated blasts, exhibiting one or more type of early myeloid differentiation. AML generally evolves in the bone marrow and, to a lesser degree, in the secondary hematopoietic organs. AML primarily affects adults, peaking in incidence between the ages of 15-40, but it is also known to affect both children and older adults. Nearly all patients with AML require treatment immediately after diagnosis to achieve clinical remission, in which there is no evidence of abnormal levels of circulating undifferentiated blast cells.

To date, a variety of MAbs have been developed that induce cytolytic activity against tumor cells. The murine MAb muMab4D5 produced against the extracellular domain of HEC2 (P185) and found to markedly inhibit the proliferation of human tumor cells over-expressing HER2 was humanized to produce the drug HERCEPTIN® (trastuzumab), which was approved by the FDA and is being used to treat human breast cancer (U.S. Pat. Nos. 5,821,337 and 5,720,954). Following binding, the antibody is capable of inhibiting tumor cell growth that is dependent on the HER2 growth factor receptor. In addition, a chimeric antibody against CD20, Rituxan® (rituximab), which causes rapid depletion of peripheral B cells, including those associated with lymphoma, was recently approved by the FDA (U.S. Pat. No. 5,843,439). The binding of this antibody to target cells results in complement-dependent lysis. This product has recently been approved and is currently being used in the clinic to treat low-grade B cell non-Hodgkin's lymphoma.

Several other humanized and chimeric antibodies are under development or are in clinical trials. In addition, a humanized immunoglobulin (Ig) that specifically reacts with CD33 antigen, expressed both on normal myeloid cells as well as on most types of myeloid leukemic cells, was conjugated to the anti-cancer drug calicheamicin, CMA-676 (Sievers et al., Blood Supp. 308: 504a (1997)). This conjugate, known as the drug MYLOTARG®, has recently received FDA approval (Caron et al. Cancer Supp. 73: 1049-56 (1994)). In light of its cytolytic activity, an additional anti-CD33 antibody (HumM195), currently in clinical trials, was conjugated to several cytotoxic agents, including the gelonin toxin (McGraw et al., Cancer Immunol. Immunother. 39: 367-74 (1994)) and radioisotopes ¹³¹I (Caron et al., Blood 83: 1760-68 (1994)), ⁹⁰Y (Jurcic et al., Blood Supp. 92: 613a (1998)) and ²¹³Bi (Humm et al., Blood Supplement 38: 231P (1997)).

A chimeric antibody against the leukocyte antigen CD45 (cHuLym3) is in clinical studies for treatment of human leukemia and lymphoma (Sun et al., Cancer Immunol. Immunother. 48: 595-602 (2000)). In in vitro assays, specific cell lysis was observed in ADCC (Antibody Dependent Cell-mediated Cytotoxicity) assays (Henkart, Immunity 1: 343-46 (1994); Squier and Cohen, Current Opin. Immunol. 6: 447-52 (1994)).

Therapeutic antibodies have also been specifically engineered to have higher affinity to their target, to be more stable, and for optimal biodistribution. See, e.g., Presta, Current Pharma. Biotechnol., 3: 237-56 (2002); Presta et al., Biochem. Society Transactions, 30(4): 487-90 (2002).

In contrast to mouse monoclonal humanization and construction of chimeric antibodies, the use of phage display technology enables the isolation of scFvs having fully human sequences. A fully human antibody against the human TGFα-2 receptor based on an scFv clone derived from phage display technology was recently developed. This scFv, which was converted into a fully human IgG4 capable of competing with the binding of TGFα-2 (Thompson et al., J. Immunol. Meth. 227: 17-29 (1999)), has strong anti-proliferative activity. Phage display technology, as known to one skilled in the art, is more specifically described in the following publications: Smith, Science 228: 1315 (1985); Scott et al., Science 249: 386-90 (1990); Cwirla et al., PNAS 87: 6378-82 (1990); Devlin et al., Science 249: 404-06 (1990); Griffiths et al., EMBO J. 13(14): 3245-60 (1994); Bass et al., Proteins 8: 309-14 (1990); McCafferty et al., Nature 348: 552-54 (1990); Nissim et al., EMBO J. 13: 692-98 (1994); U.S. Pat. Nos. 5,427,908, 5,432,018, 5,223,409 and 5,403,484.

Ligand for Isolated scFv Antibody Molecules

Platelets, fibrinogen, GPIb, selecting, and PSGL-1 (P-Selectin Glycoprotein Ligand-1) each play an important role in several pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that cross-react with platelets and with these molecules would be useful in the diagnosis and treatment of diseases and disorders involving these and other pathogenic conditions.

### Platelets

Platelets are well-characterized components of the blood system and play several important roles in hemostasis, thrombosis and/or restenosis. Damage to blood vessels sets in motion a process known as hemostasis, which is characterized by a series of sequential events. The initial reaction to damaged blood vessels is the adhesion of platelets to the affected region on the inner surface of the vessel. The next step is the aggregation of many layers of platelets onto the previously adhered platelets, forming the hemostatic plug and scaling the vessel wall. The hemostatic plug is further strengthened by the deposition of fibrin polymers. The clot or plug is degraded only when the damage has been repaired. Circulating platelets are cytoplasmic particles released from the periphery of megakaryocytes. Platelets thus play an important role in hemostasis. Upon vascular injury, platelets adhere to damaged tissue surfaces and attach one another (cohesion). This sequence of events occurs rapidly, forming a structureless mass (commonly called a platelet plug or thrombus) at the site of vascular injury. The cohesion phenomenon, also known as aggregation, may be initiated in vitro by a variety of substances, or agonists, such as collagen, adenosine diphosphate (ADP), epinephrine, serotonin, and ristocetin. Aggregation is one of the numerous in vitro tests performed as a measure of platelet function.

### Importance of Platelets in Metastasis

Tumor metastasis is perhaps the most important factor limiting the survival of cancer patients. Accumulated data indicate that the ability of tumor cells to interact with host platelets represents one of the indispensable determinants of metastasis (Oleksowicz, Thrombosis Res. 79: 261-74 (1995)).

It has been demonstrated that the ability of tumor cells to aggregate platelets correlates with the tumor cells' metastasis potential, and inhibition of tumor-induced platelet aggregation has been shown to correlate with the suppression of metastasis in rodent models. It has been demonstrated that tumor cell interaction with platelets involves membrane adhesion molecules and agonist secretion. Expression of immuno-related platelet glycoproteins has been identified on tumor cell lines. It was demonstrated that platelet immuno-related glycoproteins, GPIb, GPIIb/IIIa, GPIb/IX and the integrin αᵥ subunit are expressed on the surface of breast tumor cell lines (Oleksowicz, (1995), supra; Kamiyama et al., J. Lab. Clin. Med. 117(3): 209-17 (1991)).

Gasic et al. (PNAS 61:46-52 (1968)) showed that antibody-induced thrombocytopenia markedly reduced the number and volume of metastases produced by CT26 colon adenocarcinoma, Lewis lung carcinoma, and B16 melanoma (Karpatkin et al., J. Clin. Invest. 81(4): 1012-19 (1988); Clezardin et al., Cancer Res. 53(19): 4695-700 (1993)). Furthermore, a single polypeptide chain (60 kd) was found to be expressed on surface membrane of HEL cells which is closely related to GPIb and corresponds to an incompletely or abnormally O-glycosylated GPIbα subunit (Kieffer et al., J. Biol. Chem. 261(34): 15854-62 (1986)).

Platelets are also involved in the process of metastasis. When metastatic cancer cells enter the blood stream, multicellular complexes composed of platelets and leukocytes coating the tumor cells are formed. These complexes, which may be referred to as microemboli, aid the tumor cells in evading the immune system. The coating of tumor cells by platelets requires expression of P-selectin by the platelets.

### GPIb Complex

Each step in the process of hemostasis requires the presence of receptors on the platelet surface. One receptor that is important in hemostasis is the glycoprotein Ib-IX complex (also known as CD42). This receptor mediates adhesion (initial attachment) of platelets to the blood vessel wall at sites of injury by binding von Willebrand factor (vWF) in the subendothelium. It also has crucial roles in two other platelet functions important in hemostasis: (a) aggregation of platelets induced by high shear in regions of arterial stenosis and (b) platelet activation induced by low concentrations of thrombin.

The GPIb-IX complex is one of the major components of the outer surface of the platelet plasma membrane. The GPIb-IX complex comprises three membrane-spanning polypeptides—a disulfide-linked 130 kDa α-chain and 25 kDa β-chain of GPIb and a noncovalently associated GPIX (22 kDa). All of the subunits are presented in equimolar amounts on the platelet membrane, for efficient cell-surface expression and function of CD42 complex, indicating that proper assembly of the three subunits into a complex is required for full expression on the plasma membrane. The α-chain of GPIb consists of three distinct structural domains (1) a globular N-terminal peptide domain containing leucine-rich repeat sequences and Cys-bonded flanking sequences; (2) a highly glycosylated mucin-like macroglycopeptide domain; and (3) a membrane-associated C-terminal region that contains the disulfide bridge to GPIbα transmembrane and cytoplasmic sequences.

Several lines of evidence indicate that the vWF and thrombin-binding domain of the GPIb-IX complex reside in a globular region that encompasses approximately 300 amino acids at the amino terminus of GPIbα. The human platelet GPIb-IX complex is a key membrane receptor mediating both platelet function and reactivity. Recognition of subendothelial-bound vWF by GPIb allows platelets to adhere to damaged blood vessels. Further, binding of vWF to GPIbα also induces platelet activation, which may involve the interaction of a cytoplasmic domain of the GPIb-IX with cytoskeleton or phospholipase A2. Moreover, GPIbα contains a high-affinity binding site for α-thrombin, which facilitates platelet activation by an as-yet poorly defined mechanism.

### Selectins and PSGL-1

The P-, E-, and L-Selectins are members of a family of adhesion molecules that, among other functions, mediate rolling of leukocytes on vascular endothelium. P-Selectin is stored in granules in platelets and is transported to the surface after activation by thrombin, histamine, phorbol ester, or other stimulatory molecules. P-Selectin is also expressed on activated endothelial cells. E-Selectin is expressed on endothelial cells, and L-Selectin is expressed on neutrophils, monocytes, T cells, and B cells.

PSGL-1 (also called CD162) is a mucin glycoprotein ligand for P-Selectin, E-Selectin, and L-Selectin that shares structural similarity with GPIb (Afshar-Kharghan et al. (2001), supra). PSGL-1 is a disulfide-linked homodimer that has a PACE (Paired Basic Amino Acid Converting Enzymes) cleavage site. The extracellular portion of PSGL-1 contains three N-linked glycosylation sites and has numerous sialyated, fucosylated O-linked oligosaccharide branches (Moore et al., J. Biol. Chem. 118: 445-56 (1992)). Most of the N-glycan sites and many of the O-glycan sites are occupied. The structures of the O-glycans of PSGL-1 from human HL-60 cells have been determined. Subsets of these O-glycans are core-2, sialylated and fucosylated structures that are required for binding to selecting.

PSGL-1 has 361 residues in HL60 cells, with a 267 residue extracellular region, 25 residue trans-membrane region, and a 69 residue intracellular region. PSGL-1 forms a disulfide-bonded homodimer or heterodimer on the cell surface (Afshar-Kharghan et al., Blood 97: 3306-12 (2001)). The sequence encoding PSGL-1 is in a single exon, so alternative splicing should not be possible. However, PSGL-1 in HL60 cells, and in most cell lines, has 15 consecutive repeats of a 10 residue consensus sequences present in the extracellular region, although there are 14 and 16 repeats of this sequence in polymorphonuclear leukocytes, monocytes, and several other cell lines, including most native leukocytes.

PSGL-1 is expressed on neutrophils as a dimer, with apparent molecular weights of both 250 kDa and 160 kDa, whereas on HL60 the dimeric form is approximately 220 kDa. When analyzed under reducing conditions, each subunit is reduced by half Differences in molecular mass may be due to polymorphisms in the molecule caused by the presence of different numbers of decamer repeats (Leukocyte Typing VI. Edited by T. Kishimoto et al. (1997)).

PSGL-1 is also expressed on most blood leukocytes, such as neutrophils, monocytes, leukocytes, subset of B cells, and all T cells (Kishimoto et al. (1997), supra). PSGL-1 mediates rolling of leukocytes on activated endothelium, on activated platelets, and on other leukocytes and inflammatory sites and mediates rolling of neutrophils on P-Selectin. PSGL-1 may also mediate neutrophil-neutrophil interactions via binding with L-Selectin, thereby mediating inflammation (Snapp et al., Blood 91(1): 154-64 (1998)).

Leukocyte rolling is important in inflammation, and interaction between P-Selectin (expressed by activated endothelium and on platelets, which may be immobilized at sites of injury) and PSGL-1 is instrumental for tethering and rolling of leukocytes on vessel walls (Ramachandran et al., PNAS 98(18): 10166-71 (2001); Afshar-Kharghan et al. (2001), supra). Cell rolling is also important in metastasis, and P- and E-Selectin on endothelial cells is believed to bind metastatic cells, thereby facilitating extravasation from the blood stream into the surrounding tissues.

Thus, PSGL-1 has been found on all leukocytes: neutrophils, monocytes, lymphocytes, activated peripheral T cells, granulocytes, eosinophils, platelets, and on some CD34 positive stem cells and certain subsets of B cells. P-Selectin is selectively expressed on activated platelets and endothelial cells. Interaction between P-Selectin and PSGL-1 promotes rolling of leukocytes on vessel walls, and abnormal accumulation of leukocytes at vascular sites results in various pathological inflammations. Stereo-specific contributions of individual tyrosine sulfates on PSGL-1 are important for the binding of P-Selectin to PSGL-1. Charge is also important for binding: reducing NaCl (from 150 to 50 mM) enhanced binding (Kd ˜75 nM). Tyrosine-sulfation on PSGL-1 enhances, but is not ultimately required for PSGL-1 adhesion on P-Selectin. PSGL-1 tyrosine sulfation supports slower rolling adhesion at all shear rates and supports rolling adhesion at much higher shear rates (Rodgers et al., Biophys. J. 81: 2001-09 (2001)). Moreover, it has been suggested that PSGL-1 expression on platelets is 25-100 fold lower than in leukocytes. Frenette et al., J. Exp. Med. 191(8): 1413-22 (2000)).

A commercially available monoclonal antibody to human PSGL-1, KPL1, was generated and shown to inhibit the interactions between PSGL-1 and P-selectin and between PSGL-1 and L-selectin. The KPL1 epitope was mapped to the tyrosine sulfation region of PSGL-1 (YEYLDYD) (SEQ ID NO:1) (Snapp et al., Blood 91(1):154-64 (1998)).

Pretreatment of tumor cells with O-sialoglycoprotease, which removes sialylated, fucosylated mucin ligands, also inhibited tumor cell-platelet complex formation. In vivo experiments indicate that either of these treatments results in greater monocyte association with circulating tumor cells, suggesting that reducing platelet binding increases access by immune cells to circulating tumor cells (Varki and Varki, Braz. J. Biol. Res. 34(6): 711-17 (2001)).

### Fibrinogen

There are two forms of normal human fibrinogen—normal (γ) and γ′, each of which is found in normal individuals. Normal fibrinogen, which is the more abundant form (approximately 90% of the total fibrinogen found in the body), is composed of two identical 55 kDa β chains, two identical 95 kDa β chains, and two identical 49.5 kDa γ chains. Normal variant fibrinogen, which is the less abundant form (approximately 10% of the fibrinogen found in the body), is composed of two identical 55 kDa β chains, two identical 95 kDa β chains, one 49.5 kDa γ chain, and one variant 50.5 kDa γ′ chain. The gamma and gamma prime chains are both coded for by the same gene, with alternative splicing occurring at the 3′ end. Normal gamma chain is composed of amino acids 1-411 and normal variant gamma prime chain is composed of 427 amino acids, of which amino acids 1-407 are the same as those in the normal gamma chain and amino acids 408-427 are VRPEHPAETEYDSLYPEDDL (SEQ ID NO:2). This region is normally occupied with thrombin molecules.

Fibrinogen is converted into fibrin by the action of thrombin in the presence of ionized calcium to produce coagulation of the blood. Fibrin is also a component of thrombi, and acute inflammatory exudates.

### HIV Infectivity

To trigger the membrane fusion process that leads to viral entry, HIV-1 must first interact with CD4 then with a co-receptor. CD4 binding occurs subsequent to less specific, adhesion factor-mediated interactions with the cell surface that increase the localized concentration of virions. Binding of the HIV-1 gp120 envelope glycoprotein to CD4 induces conformational changes in gp120 that create or expose a binding site for a co-receptor. Once available, the co-receptor binding site interacts with a complex, discontinuous region of the co-receptor that involves, but is not limited to the amino-terminal domain (Nt). The association of gp120 with CCR5 or CXCR4 then drives additional conformational changes within the entire trimeric gp120/gp41 complex that eventually lead to the insertion of the gp41 fusion peptide into the host cell membrane, provoking fusion and entry. See Cormier, E. G. and Dragic, T. “An overview of HIV-1 co-receptor function and its inhibitors” HIV database review articles (2000).

HIV-1 co-receptors belong to the seven transmembrane G-protein coupled chemokine receptor family. The evidence accumulated to date: indicates that there are similarities and differences in the way HIV-1 envelope glycoproteins from R5 and X4 isolates interact with their respective co-receptors. A cluster of residues in the CCR5 Nt participates in gp120-binding and is essential for fusion and entry of both R5 and R5X4 isolates. In contrast, residues dispersed throughout the extracellular domain of CXCR4 are involved in gp120 docking, viral fusion and entry; each HIV-1 isolate uses a slightly different subset of CXCR4 residues in order to gain entry into the target cell. Nevertheless, the gp120 binding sites on CCR5 and CXCR4 comprise negatively charged and tyrosine residues. Certain mutations in CXCR4 even enable it to mediate the entry of R5 isolates. Similarities between CCR5 and CXCR4 gp120-binding sites are further underscored by the ability of R5X4 isolates to interact with both co-receptors. These similarities may account for the ability of a few residue changes in gp120 to induce a switch in co-receptor usage. It should be noted that the extracellular loops of CCR5 and CXCR4 also play an indirect role in viral entry by influencing the overall conformation and/or oligomerization of the co-receptor proteins. It is notable, however, that all chemokine receptors described to date have negatively charged regions in their extracellular domains, yet most do not mediate HIV-1 entry, and some do so only poorly. It also seems that the Nts of most if not all chemokine receptors contain sulfotyrosines. Hence, the unique features that make CCR5 and CXCR4 efficient HIV-1 co-receptors remain to be identified. Perhaps it is the way that the different Tyr-Asp-Glu motifs are exhibited on the surfaces of these receptors, or the ability of CCR5 and CXCR4 to interact with CD4 or other molecules on the cell surface that ultimately renders them efficient mediators of viral entry. See id.

The selective use of the CCR5 and/or CXCR4 co-receptors is the molecular explanation of the previous phenotypic categorization of HIV-1 isolates. CCR5 is the principal co-receptor for HIV variants that are sexually transmitted and persist within the majority of infected individuals (R5 isolates). The appearance of variants that use CXCR4 or both co-receptors (X4 and R5X4 isolates) signals accelerated CD4+ T-cell loss and disease progression. The phenotypic switch from R5 to X4 viruses in vivo typically occurs only after several years of infection. This is surprisingly slow given that changing only a few residues in gp120 can be sufficient to convert an R5 virus into an RSX4 virus in vitro and that such changes must be occurring continuously given the error rate of reverse transcription. These observations imply that the transition to CXCR4 usage is specifically suppressed in vivo. See id.

Among the many chemokine receptors that can mediate HIV-1 entry in vitro, it is believed that only CCR5 and CXCR4 are of pharmacological importance, since they are the principal co-receptors used by HIV-1 to enter primary CD4+ T-cells and macrophages. See id.

The first inhibitors known to prevent HIV-1 fusion and entry were MIP-1α, MIP-1β and RANTES, the natural CC-chemokine ligands of CCR5. The CXC-chemokine SDF-1α has an analogous inhibitory effect on viral entry via CXCR4. Variants of chemokines with increased potency in vitro, usually resulting from N-terminal modifications to the RANTES or SDF-1α structure, have since been developed. Chemokines interfere with HIV-1 replication by several mechanisms: (1) direct competition between the chemokine and the gp120 glycoprotein for binding to the co-receptor; (2) a sustained down-regulation of the co-receptor as a consequence of chemokine binding and signal transduction; and (3) alteration of the differentiation state of the target cell that affects HIV-1 replication late in the viral life cycle. See id.

Several CXCR4- and CCR5-specific murine MAbs are known to inhibit HIV-1 fusion and entry with considerable potency. Co-receptor specific MAbs are not agonists, but most are antagonists. It has been shown that anti-CCR5 MAbs that recognize epitopes in the second extracellular loop (ECL2) are potent inhibitors of HIV-1 entry even though they only moderately inhibit gp120 binding to CCR5. Possibly, these MAbs inhibit important postgp120 binding steps, such as conformational changes in CCR5 or its oligomerization. Few anti-CXCR4 MAbs have been generated and only one has been extensively characterized. MAb 12G5 recognizes an epitope in ECL2 and inhibits HIV-1 fusion and entry both in an isolate- and a cell type specific manner. Differences in gp120 affinities for CXCR4 and post-translational modifications of CXCR4 in different cell types could account for these discrepancies. Other anti-CXCR4 MAbs, whose epitopes remain to be determined, also variably inhibit the entry of the HIV-1 NL-43 isolate. See id.

### Protein Sulfation

Protein sulfation is a widespread post-translational modification that involves enzymatic covalent attachment of sulfate, either to sugar side chains or to the polypeptide backbone. This modification occurs in the trans-Golgi compartment. Such proteins include secretory proteins, proteins targeted for granules, and the extracellular regions of plasma membrane proteins. Tyrosine is an amino acid residue presently known to undergo sulfation. Kehoe et al., Chem. Biol. 7: R57-61 (2000). Other amino acids, e.g., threonine, may also undergo sulfation, particularly in diseased cells.

With respect to GPIb, the negatively-charged N-terminal globular domain of GPIb contains three tyrosine residues known to undergo sulfation. GPIbα (CD42), which is expressed by platelets and megakaryocytes and mediates platelet attachment to and rolling on subendothelium via binding with vWF, also contains a cluster of negatively charged amino acids between Asp-269 and Asp-287. Such a highly acidic and hydrophilic environment is thought to be a prerequisite for sulfation, because tyrosylprotein sulfotransferase specifically recognizes and sulfates tyrosines adjacent to acidic amino residues (Bundgaard et al., J. Biol. Chem. 272:21700-05 (1997)). Full sulfation of the acidic region of GPIbα yields a region with remarkable negative charge density—13 negative charges within a 19 amino acid stretch, making it a candidate site for electrostatic interaction with other proteins. Several lines of evidence indicate that, in transfected CHO cells expressing GPIb-IX complex and in platelet GPIbα, the three tyrosine residues contained in this domain (Tyr-276, Tyr-278, and Tyr-279) undergo sulfation.

With respect to PSGL-1, this protein has three potential sulfation sites (on each of the three tyrosine residues at the N-terminal domain of the molecule) followed by 10-16 decamer repeats that are high in proline, serine, and threonine.

Sulfation of PSGL-1 is known to be relevant for the binding of P-selectin, and stereo-specific contributions of individual tyrosine sulfates on PSGL-1 are important for the binding of P-selectin to PSGL-1. There are some indications, however, that sulfation of only one tyrosine residue is sufficient for P-selectin binding. (J. Biol. Chem., Vol. 273, 12, 7078-87). PSGL-1 tyrosine sulfation supports slower rolling adhesion at all shear rates and supports rolling adhesion at much higher shear rates (Rodgers et al., Biophys. J. 81: 2001-09 (2001)).

It is also thought that sulfated N-terminal tyrosines influence the role of CC-chemokine receptors, such as CCR5, which serve as co-receptors with related seven transmembrane segment (7TMS) receptor for entry of human and simian immunodeficiency viruses (HIV-1, HIV-2, and SIV) into target cells. For example, it is thought that sulfated N-terminal tyrosines contribute to the binding of CCR5 to MIP-1α, MIP-1β, and HIV-1 gp120/CD4 complexes and to the ability of HIV-1 to enter cells expressing CCR5 and CD4. CXCR4, another important HIV-1 co-receptor, is also sulfated (Farzan et al., Cell 96(5): 667-76 (1999)). Tyrosine sulfation plays a less significant role in CXCR4-dependent HIV-1 entry than CCR5-dependent entry; thus demonstrating a possible role for tyrosine sulfation in the CXC-chemokine family and underscores a general difference in HIV-1 utilization of CCR5 and CXCR4 (Farzan et al., J. Biol. Chem., 277(33): 29, 484-89 (2002)).

Antibodies that bind to PSGL-1 and/or GPIb were identified using a phage library and disclosed in U.S. application Ser. Nos. 10/032,423; 10/032,037; 10/029,988; 10/029,926; 09/751,181; 10/189,032; and 60/258,948 and International Application Nos. PCT/US01/49,442 and PCT/US01/49,440. Specific examples of antibodies disclosed in these applications include the Y1, Y17, and L32 antibodies. These antibodies were isolated from the germ line (DP32) and were discovered to specifically bind to an epitope, found on proteins of the hematopoetic cells, which is sulfated at an N-terminal tyrosine and is thought to be involved in cell migration, e.g. tumor metastasis.

The sulfated epitopes previously identified as binding to Y1/Y17/L32 are characterized by the presence of sulfated moieties, such as sulfated tyrosine residues or sulfated carbohydrate or lipid moieties, preferably within a cluster of two or more acidic amino acids, which are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as T-ALL cells, B-CLL cells, AML cells, multiple myeloma cells, and metastatic cells.

These epitopes are useful targets for the therapeutic mediation of these processes (as well as targeting agents) and for diagnostic procedures.

### OBJECTIVES

It is an object of the present invention to provide antibodies and polypeptides that bind to epitopes that are present on various molecules instrumental in processes such as cell rolling, inflammation, immune reactions, infection, autoimmune reactions, metastasis, and HIV entry.

It is also an object of the present invention to provide antibodies and polypeptides that bind to epitopes that are involved in processes such as adhesion, thrombosis and/or restenosis and platelet aggregation and that can be used for the treatment of cardiovascular diseases such as myocardial infarction, and restenosis, and for the treatment of inflammatory diseases.

It is also an object of the present invention to provide antibodies and polypeptides that bind to chemokine receptors such as CCR5 and CXCR4, which are involved in the entry of HIV into cells. Such antibodies and polypeptides are believed to inhibit entry of HIV into cells and thus provide a treatment of HIV infection.

It is also an object of this invention to utilize antibodies and polypeptides in methods for diagnosing, prognosing, or staging various disease states of an individual, such as, e.g., AML, T-ALL, B-leukemia, B-CLL, Pre-B-ALL, multiple myeloma, metastasis, HIV infection, cardiovascular diseases, or other diseases in which such cellular functions or actions as cell rolling, inflammation, immune reactions, infection, autoimmune reactions, metastasis, play a significant role. In addition, these antibodies of the present invention may be used as a targeting agent to direct a therapeutic to a specific cell or site.

It is also an object of the present invention to provide methods of treatment for various disease states such as, e.g., AML, T-ALL, B-leukemia, B-CLL, Pre-B-ALL, multiple myeloma, metastasis, HIV infection, cardiovascular diseases, or other diseases in which such cellular functions or actions as cell rolling, inflammation, immune reactions, infection, autoimmune reactions, metastasis, play a significant role. And another object of the present invention is to provide a method of purging tumor cells.

It is also an object of the present invention to provide antibodies and polypeptides for use in manufacturing a medicament for the treatment of various disease states such as, e.g., AML, T-ALL, B-leukemia, B-CLL, Pre-B-ALL, multiple myeloma, metastasis, HIV infection, cardiovascular diseases including reperfusion injury and artherosclerosis, or other diseases in which such cellular functions or actions as cell rolling, inflammation, immune reactions, infection, autoimmune reactions, metastasis, play a significant role.

It is also an object of the present invention to provide antibodies and polypeptides, which stimulate T-cells, NK cells and/or stimulates antibody dependent cell cytotoxicity and/or apoptosis.

It is also an object of the present invention to provide polypeptides, expression vectors, and recombinant host cells expressing antibodies that are involved in processes such as cell rolling, inflammation, immune reactions, infection, autoimmune reactions, metastasis, HIV entry, adhesion, thrombosis restenosis and platelet aggregation.

It is a further objective of the present invention to provide a library of immunoglobulin binding domains, which specifically are scFv molecules.

These and other objectives of the invention are provided herein.

### SUMMARY OF THE INVENTION

The present invention provides an antibody and polypeptide comprising a consensus sequence: X₁-X₂-X₃-Pro-X₅-X₆ (SEQ ID NO:3) wherein X₁ and X₆ are hydrophobic amino acids, and X₂ X₃ and X₅ are any amino acid, wherein X₂ is preferably a basic amino acid, and wherein the consensus sequence is arranged from N-terminus to C-terminus or from C-terminus to N-terminus. The consensus sequence is preferably within the hypervariable regions of the antibody and more preferably within the CDR3 region. Preferably, the consensus sequence excludes a CDR3 region comprising the amino acid sequence of SEQ ID NO:4. The present invention moreover provides an antibody and polypeptide having the binding capabilities of an scFv antibody of SEQ ID NO:5 or SEQ ID. NO:6.

The present invention additionally provides a process for producing an antibody or polypeptide comprising the steps of providing a phage display library, providing a peptide of SEQ ID NO:7 that binds to an antibody or polypeptide having the binding capabilities of an scFv antibody fragment of SEQ ID NO:4, panning the phage display library for an scFv antibody fragment that binds to the peptide of SEQ ID NO:7, and producing an antibody or polypeptide comprising the scFv antibody fragment that binds to the peptide of SEQ ID NO:7.

The present invention also provides a library of immunoglobulin binding domains, specifically scFv molecules, comprising a diverse antigen-binding domain for complementary binding, wherein the library has diversity only in heavy chain CDR3.

The present invention also provides pharmaceutical compositions comprising the antibodies and polypeptides of the present invention. These pharmaceutical compositions may be used to treat, diagnose, prognose or stage various conditions including conditions related to or involving cell rolling; inflammation; auto-immune disease; platelet aggregation; restenosis; HIV infection; metastasis; growth and/or replication of tumor cells; and growth and/or replication of leukemia cells. These pharmaceutical compositions may be used for inhibiting cell rolling; inhibiting inflammation; inhibiting auto-immune disease; inhibiting platelet aggregation; inhibiting restenosis; inhibiting HIV infection; inhibiting metastasis; inhibiting growth and/or replication of tumor cells, increasing mortality of tumor cells, inhibiting growth and/or replication of leukemia cells, increasing the mortality rate of leukemia cells; altering the susceptibility of diseased cells to damage by anti-disease agents; increasing the susceptibility of tumor cells to damage by anti-cancer agents; increasing the susceptibility of leukemia cells to damage by anti-leukemia agents; inhibiting increase in number of tumor cells in a patient having a tumor; decreasing the number of tumor cells in a patient having cancer; inhibiting increase in number of leukemia cells in a patient having leukemia; and decreasing the number of leukemia cells in a patient having leukemia.

The present invention moreover provides a method of manufacturing a medicament for the treatment of various disease states such as, e.g., AML, T-ALL, B-leukemia, B-CLL cells, Pre-B-ALL, multiple myeloma, metastasis, HIV infection, cardiovascular diseases, or other diseases in which such cellular functions or actions as cell rolling, inflammation, immune reactions, infection, autoimmune reactions, metastasis, play a significant role.

The present invention also provides a method of diagnosing, prognosing, or staging a disease in a patient by providing a sample containing a cell from the patient and determining whether the antibodies or polypeptides of the present invention bind to the cell of the patient, thereby indicating that the patient is at risk for or has the disease. The present invention also provides a method of purging tumor cells from a patient by providing a sample containing cells from the patient and incubating the cells from the patient with an antibody or polypeptide of the present invention.

### DEFINITIONS

Antibodies (Abs), or immunoglobulins (Igs), are protein molecules that bind to antigen. Each functional binding unit of naturally occurring antibodies is composed of units of four polypeptide chains (2 heavy and 2 light) linked together by disulfide bonds. Each of the chains has a constant and variable region. Naturally occurring antibodies can be divided into several classes including, IgG, IgM, IgA, IgD, and IgE, based on their heavy chain component. The IgG class encompasses several sub-classes including, but not restricted to, IgG₁, IgG₂, IgG₃, and IgG₄. Immunoglobulins are produced in vivo by B-lymphocytes, and each such molecule recognizes a particular foreign antigenic determinant and facilitates clearing of that antigen.

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Examples of antibody fragments include Fv, Fab, F(ab′)₂, Fc, and Fd fragments. Therefore, an antibody according to the present invention encompasses a complex of an antibody or fragment thereof.

As used herein in the specification and in the claims, an Fv is defined as a molecule that is made up of a variable region of a heavy chain of a human antibody and a variable region of a light chain of a human antibody, which may be the same or different, and in which the variable region of the heavy chain is connected, linked, fused, or covalently attached to, or associated with, the variable region of the light chain. The Fv can be a single chain Fv (scFv) or a disulfide stabilized Fv (dsFv). An scFv is comprised of the variable domains of each of the heavy and light chains of an antibody, linked by a flexible amino-acid polypeptide spacer, or linker. The linker may be branched or unbranched. Preferably, the linker is 0-15 amino acid residues, and most preferably the linker is (Gly₄Ser)₃ (SEQ ID NO:8).

The Fv molecule, itself, is comprised of a first chain and a second chain, each chain having a first, second and third hypervariable region. The hypervariable loops within the variable domains of the light and heavy chains are termed Complementary Determining Regions (CDR). There are CDR1s, CDR2s, and CDR3s in each of the heavy and light chains. These regions are believed to form the antigen-binding site and can be specifically modified to yield enhanced binding activity. The most variable of these regions in nature is the CDR3 of the heavy chain. The CDR3 is understood to be the most exposed region of the Ig molecule and, as shown and provided herein, is the site primarily responsible for the selective and/or specific binding characteristics observed.

A fragment of a Fv molecule is defined as any molecule smaller than the original Fv that still retains the selective and/or specific binding characteristics of the original Fv. Examples of such fragments include but are limited to (1) a minibody, which comprises a fragment of the heavy chain only of the Fv, (2) a microbody, which comprises a small fractional unit of antibody heavy chain variable region (International Application No. PCT/IL99/00581), (3) similar bodies having a fragment of the light chain, and (4) similar bodies having a functional unit of a light chain variable region.

As used herein the term “Fab fragment” is a monovalent antigen-binding fragment of an immunoglobulin. A Fab fragment is composed of the light chain and part of the heavy chain.

An F(ab′)₂ fragment is a bivalent antigen binding fragment of an immunoglobulin obtained by pepsin digestion. It contains both light chains and part of both heavy chains.

An Fc fragment is a non-antigen-binding portion of an immunoglobulin. It contains the carboxy-terminal portion of heavy chains and the binding sites for the Fc receptor.

A Fd fragment is the variable region and first constant region of the heavy chain of an immunoglobulin.

Polyclonal antibodies are the product of an immune response and are formed by a number of different B-lymphocytes. Monoclonal antibodies are derived from one clonal B cell.

Hydrophobic amino acids are generally valine (V), isoleucine (I), leucine (L), methionine (M), phenylalanine (F), tryptophan (W), cysteine (C), alanine (A), tyrosine (Y), threonine (T), serine (S), proline (P), and glycine (G). Basic amino acids are generally arginine, histidine, and lysine.

A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends.

The term “epitope” is used herein to mean the antigenic determinant or recognition site or antigen site that interacts with an antibody, antibody fragment, antibody complex or a complex having a binding fragment thereof or T cell receptor. The term epitope is used interchangeably herein with the terms ligand, domain, and binding region.

Selectivity is herein defined as the ability of a targeting molecule to choose and bind one entity or cell state from a mixture of entities or entity states, all entities or entity states of which may be specific for the targeting molecule.

The term “affinity” as used herein is a measure of the binding strength (association constant) between a binding molecule (e.g., one binding site on an antibody) and a ligand (e.g., antigenic determinant). The strength of the sum total of noncovalent interactions between a single antigen-binding site on an antibody and a single epitope is the affinity of the antibody for that epitope. Low affinity antibodies bind antigen weakly and tend to dissociate readily, whereas high-affinity antibodies bind antigen more tightly and remain bound longer. The term “avidity” differs from affinity, because the former reflects the valence of the antigen-antibody interaction.

Specificity of antibody-antigen interaction: Although the antigen-antibody reaction is specific, in some cases antibodies elicited by one antigen can cross-react with another unrelated antigen. Such cross-reactions occur if two different antigens share a homologous or similar structure, epitope, or an anchor region thereof, or if antibodies specific for one epitope bind to an unrelated epitope possessing similar structure conformation or chemical properties.

A platelet is a disc-like cytoplasmic fragment of a megakaryocyte that is shed in the marrow sinus and subsequently circulates in the peripheral blood stream. Platelets have several physiological functions including a major role in clotting. A platelet contains centrally located granules and peripheral clear protoplasm, but has no definite nucleus. Agglutination as used herein means the process by which suspended bacteria, cells, discs, or other particles of similar size are caused to adhere and form into clumps. The process is similar to precipitation but the particles are larger and are in suspension rather than being in solution.

The term aggregation means a clumping of platelets induced in vitro, and thrombin and collagen, as part of a sequential mechanism leading to the formation of a thrombus or hemostatic plug.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:
glycine (G), alanine (A), valine (V), leucine (L) and isoleucine (I)
aspartic acid (D) and glutamic acid (E)
alanine (A), serine (S) and threonine (T)
histidine (H), lysine (K) and arginine (R)
asparagine (N) and glutamine (Q)
phenylalanine (F), tyrosine (Y) and tryptophan (W)

Conservative amino acid substitutions can be made in, e.g., regions flanking the hypervariable regions primarily responsible for the selective and/or specific binding characteristics of the molecule, as well as other parts of the molecule, e.g., variable heavy chain cassette. Additionally or alternatively, modification can be accomplished by reconstructing the molecules to form full-size antibodies, diabodies (dimers), triabodies (timers), and/or tetrabodies (tetramers) or to form minibodies or microbodies.

A promoter is a region on DNA at which RNA polymerase binds and initiates transcription.

A phage display library (also termed phage peptide/antibody library, phage library, or peptide/antibody library) comprises a large population of phages (108 or larger), each phage particle displaying a peptide sequence.

A pharmaceutical composition refers to a formulation which comprises a peptide or polypeptide of the invention and a pharmaceutically acceptable carrier, excipient or diluent thereof, or an antibody-pharmaceutical agent (antibody-agent) complex and a pharmaceutically acceptable carrier, excipient or diluent thereof.

An agent refers to an agent that is useful in the treatment of active disease, prophylactic treatment, or diagnosis of a mammal including, but not restricted to, a human, bovine, equine, porcine, murine, canine, feline, or any other warm-blooded animal. The agent is selected from the group of radioisotope, toxin, oligonucleotide, recombinant protein, antibody fragment, pharmaceutical agents, anti-cancer agents, anti-leukemic agents, anti-metastasis agents, anti-neoplastic agents, anti-disease agents, anti-adhesion agents, anti-thrombosis agents, anti-restenosis agents, anti-autoimmune agents, anti-aggregation agents, anti-bacterial agents, anti-viral agents, and anti-inflammatory agents. Other examples of such agents include, but are not limited to anti-viral agents including acyclovir, ganciclovir, and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid, and derivatives, combinations and modifications thereof.

An anti-leukemia agent is an agent with anti-leukemia activity. For example, anti-leukemia agents include agents that inhibit or halt the growth of leukemic or immature pre-leukemic cells, agents that kill leukemic or pre-leukemic cells, agents that increase the susceptibility of leukemic or pre-leukemic cells to other anti-leukemia agents, and agents that inhibit metastasis of leukemic cells. In the present invention, an anti-leukemia agent may also be an agent with anti-angiogenic activity that prevents, inhibits, retards or halts vascularization of tumors.

An anti-cancer agent is an agent with anti-cancer activity. For example, anti-cancer agents include agents that inhibit or halt the growth of cancerous or immature pre-cancerous cells, agents that kill cancerous or pre-cancerous, agents that increase the susceptibility of cancerous or pre-cancerous cells to other anti-cancer agents, and agents that inhibit metastasis of cancerous cells. In the present invention, an anti-cancer agent may also be agent with anti-angiogenic activity that prevents, inhibits, retards, or halts vascularization of tumors.

The expression pattern of a gene can be studied by analyzing the amount of gene product produced under various conditions, at specific times, in various tissues, etc. A gene is considered to be “over-expressed” when the amount of gene product is higher than that found in a normal control, e.g., non-diseased control.

A given cell may express on its surface a protein having a binding site (or epitope) for a given antibody, but that binding site may exist in a cryptic form (e.g. be sterically hindered or be blocked, or lack features needed for binding by the antibody) in the cell in a state, which may be called a first stage (stage I). Stage I may be, e.g., a normal, healthy, non-diseased status. When the epitope exists in cryptic form, it is not recognized by the given antibody, i.e., there is no binding of the antibody to this epitope or to the given cell at stage I. However, the epitope may be exposed by, e.g., undergoing modifications itself, or being unblocked because nearby or associated molecules are modified or because a region undergoes a conformational change. Examples of modifications include changes in folding, changes in post-translational modifications, changes in phospholipidation, changes in sulfation, changes in glycosylation, and the like. Such modifications may occur when the cell enters a different state, which may be called a second stage (stage II). Examples of second states, or stages, include activation, proliferation, transformation, or in a malignant status. Upon being modified, the epitope may then be exposed, and the antibody may bind.

Peptido-mimetics (peptide mimetics) are molecules that no longer contain any peptide bonds, i.e., amide bonds, between amino acids; however, in the context of the present invention, the term peptide mimetic is intended to include molecules that are no longer completely peptidic in nature, such as pseudo-peptides, semi-peptides and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. These molecules include small molecules, lipids, polysaccharides, or conjugates thereof.

### BRIEF DESCRIPTION OF THE DRAWINGS

FIG. 1 depicts the numerical data from phage ELISA of selected clones according to the present invention to analyze binding to PSGL-1

FIG. 2 depicts numerical data from scFv ELISA of selected clones according to the present invention to analyze binding to PSGL-1.

FIG. 3 depicts numerical data from FACS analysis of scFv from selected clones according to the present invention and L32 to analyze binding to ML-2 cells that express PSGL-1.

FIG. 4 depicts numerical data from ELISA of scFv from selected clones according to the present invention and L32 to analyze binding to glycocalicin.

FIG. 5 depicts numerical data from FACS analyses of scFv from selected clones according to the present invention and L32 to analyze binding to platelets and granulocytes.

FIG. 6 shows a comparison of the granulocyte/platelet binding ratio of selected clones according to the present invention and L32.

FIG. 7 depicts results analyzing the binding of S15 to ML-2 cells in the presence and absence of KPL-1.

FIG. 8 depicts numerical data from FACS analyses providing a comparison of the binding of purified scFvs to ML-2 cells in PBS.

FIG. 9 depicts numerical data from FACS analyses providing a comparison of the binding of purified scFvs to ML-2 cells in PBS.

FIG. 10 depicts numerical data from FACS analyses providing a comparison of the binding of purified scFvs to ML-2 cells in 50% plasma.

FIG. 11 depicts numerical data from FACS analyses providing a comparison of the binding of purified scFvs to ML-2 cells in 50% plasma.

FIG. 12 depicts a FACS analysis of the dose response of purified scFvs to ML-2 cells.

FIG. 13 depicts the binding of selected phage clones to GPIb and PSGL-1 sulfated peptides.

FIG. 14 shows binding of scFvs to glycocalicin.

FIG. 15 is a graph of binding of various scFvs at increasing concentrations to washed platelets using flow cytometry

FIG. 16 depicts binding of various scFvs to glycocalicin via an ELISA assay.

FIG. 17 depicts the effect of A3R scFv on platelet aggregation induced by ristocetin.

FIG. 18 depicts the effect of Y1 scFv, A3R scFv and control PBS on platelet adhesion to polystyrene using CPA assay.

FIG. 19 depicts the level of A3R scFv bound to guinea pig platelets following bolus injection.

FIG. 20 depicts plasma concentration of A3R scFv in guinea pig following bolus injection.

FIG. 21 depicts numerical data from direct binding of scFv to peptides based on sulfated regions of PSGL-1, GPIb and CCR5.

FIG. 22 depict S15 IgG induced ADCC in B-CLL patient samples.

FIG. 23 depicts involvement of different effector cell populations in S15 IgG induced ADCC in B-CLL patient samples.

FIG. 24 depicts the ability of S15 IgG and rituximab to induce apoptosis in B-CLL patient samples.

FIG. 25 shows the ability of scFv S11 to bind with high affinity to cells expressing CXCR4.

FIG. 26 shows the ability of scFv S11 to bind with high affinity to cells expressing CXCR4.

FIG. 27 shows that the ability of scFv S11 to bind to CXCR4 depends upon tyrosine sulfation of CXCR4.

FIG. 28 shows that binding of CXCR4 by scFv S11 specifically requires the N-terminal residue tyr-21.

### DETAILED DESCRIPTION OF THE INVENTION

The present invention relates to an antibody or fragment thereof comprising a consensus sequence: X₁-X₂-X₃-Pro-X₅-X₆ (SEQ ID NO:3), wherein X₁ and X₆ are hydrophobic amino acids and X₂, X₃ and X₅ are any amino acid, wherein X₂ is preferably a basic amino acid, and wherein the consensus sequence can be arranged either from the N-terminus to the C-terminus or from the C-terminus to the N-terminus (such antibody generally referred to herein as the consensus antibody).

In one embodiment of the consensus antibody of the present invention, X₂ is selected from the group consisting of arginine and lysine and X₁ and X₆ are selected from the group consisting of leucine, valine, methionine, alanine, phenylalanine, and isoleucine. Preferably, the consensus antibody of this embodiment comprises a consensus sequence selected from the group consisting of SEQ ID NO:9 and SEQ ID NO:10. More preferably, in this embodiment, the consensus antibody comprises SEQ ID NO:5 (the CDR3 sequence is SEQ ID NO:9) and such consensus antibody is designated and referred to herein as S15. Alternatively, the consensus antibody in this embodiment more preferably is SEQ ID NO:6 (the CDR3 sequence is SEQ ID NO:10) and such consensus antibody is designated and referred to herein as A3R.

In another embodiment of the consensus antibody of the present invention, X₂ and X₃ are arginine and the hydrophobic amino acid X₆ is preferably isoleucine. Preferably, the consensus antibody of this another embodiment comprises a consensus sequence selected from the group consisting of SEQ ID NO:9 and SEQ ID NO:10. More preferably, the consensus antibody of this another embodiment is A3R and/or S15.

In another embodiment of the consensus antibody of the present invention, X₁ is selected from the group consisting of leucine and methionine, X₂ and X₃ are arginine, X₅ is selected from the group consisting of serine and valine and X₆ isoleucine. Preferably, the consensus antibody of this another embodiment comprises a consensus sequence selected from the group consisting of SEQ ID NO:9 and SEQ ID NO:10.

In another embodiment of the consensus antibody of the present invention, X₁ is leucine, X₂ is selected from the group consisting of a basic amino acid and X₆ is selected from the group of hydrophobic amino acids. Preferred consensus antibodies of this embodiment comprises D series antibodies of SEQ ID NO:11 to SEQ ID NO:16. More preferably, the consensus antibody of this embodiment is D1 (the CDR3 region is SEQ ID NO:14 and the full scFv is SEQ ID NO:55) or D3 (the CDR3 region is SEQ ID NO:13 and the full scFv is SEQ ID NO:56).

The consensus antibody of the present invention preferably binds preferentially a first epitope over a second epitope, wherein at least one of the first and second epitope is sulfated. Examples of the first and second epitope include a PSGL-1 epitope and a GPIb epitope. More preferably, the consensus antibody binds epitopes of both PSGL-1 and GPIb and preferably shows binding with stronger affinity to a PSGL-1 epitope over a GPIb epitope or binding with stronger affinity to a GPIb epitope over a PSGL-1 epitope. Alternatively, the consensus antibody binds the first and second epitopes (e.g., PSGL-1 and GPIb) with a similar affinity. Examples of suitable antibodies that bind to PSGL-1 and GPIb with similar affinity include D1 and D3, either the full scFv or the CDR3 region.

In embodiments where the consensus antibody is A3R, preferably the consensus antibody binds an epitope of sulfated GPIb with stronger affinity than its binding to a sulfated PSGL-1 epitope. Specifically, with respect to a comparison of the binding properties of S15 and A3R at similar concentrations, it has been found that A3R binds to healthy GPIb platelets with stronger affinity than S15. It has also been found that at similar concentrations S15 binds to healthy whole blood cells (containing granulocytes; lymphocytes; and monocytes which express PSGL-1) with stronger affinity than A3R.

Thus, the present invention provides an antibody that binds specifically to sulfated PSGL-1 and/or sulfated GPIb with an affinity substantially similar to that of S15 and preferably binds to PSGL-1 with a stronger affinity than to GPIb. More preferably the antibody binds to a sulfated PSGL-1 epitope that is sulfated at the third N-terminal tyrosine residue at position 51. In another embodiment, the antibody of the present invention binds specifically to sulfated PSGL-1 and/or sulfated GPIb with an affinity substantially similar to that of A3R and preferably binds to GPIb with a stronger affinity than to PSGL-1. More preferably, the antibody of this embodiment binds to a sulfated GPIb epitope that is sulfated at the first N-terminal tyrosine residue at position 46. Alternatively, the present invention provides an antibody that binds specifically to sulfated PSGL-1 (preferably sulfated at the third N-terminal tyrosine residue at Tyr-51) and to sulfated GPIb (preferably sulfated at the first N-terminal tyrosine at Tyr-276) with an affinity substantially similar to that of D1 and/or D3

As a result of the difference in the affinity of the various antibodies of the present invention (e.g., S15 and A3R) to sulfated PSGL-1 and GPIb and because these two antibodies differ in positions X₁ and X₅ of the consensus sequence, the X₁ and X₅ of the consensus sequence of the consensus antibody of the present invention may contribute to the binding of the consensus antibody to tyrosine sulfation sites. Accordingly, the amino acids in the X₁ and X₅ positions of the consensus sequence may be specifically selected depending on the particular sulfated epitope is targeting for binding. In other words, positions X₁ and X₅ may be altered to tailor an antibody to specifically bind to a particular sulfated epitope. Furthermore, because S15 preferably binds to a PSGL-1 epitope comprising sulfate modification at the third N-terminal tyrosine and A3R preferably binds to a GPIb epitope having a sulfate modification at the first N-terminal tyrosine (while D1 and D3 bind both PSGL-1 and A3R equally), X₁ and X₅ of the consensus sequence may be relevant to binding of S15 and A3R to the third and first sulfated tyrosine of PSGL-1 and GPIb, respectively.

Antibodies that bind to PSGL-1 and/or GPIb were identified using a phage library and disclosed in U.S. application Ser. Nos. 10/032,423; 10/032,037; 10/029,988; 10/029,926; 09/751,181; 10/189,032; and 60/258,948 and International Application Nos. PCT/US01/49,442 and PCT/US01/49,440. Specific examples of antibodies disclosed in these applications include the Y1, Y17, and L32 antibodies. These antibodies were isolated from the germ line (DP32) and were discovered to specifically bind to an epitope, found on proteins of the hematopoetic cells, which is sulfated at an N-terminal tyrosine and is thought to be involved in cell migration, e.g. tumor metastasis.

The sulfated epitopes previously identified as binding to Y1/Y17/L32 are characterized by the presence of sulfated moieties, such as sulfated tyrosine residues or sulfated carbohydrate or lipid moieties, preferably within a cluster of two or more acidic amino acids, which are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as T-ALL cells, B-CLL cells, AML cells, multiple myeloma cells, and metastatic cells.

The consensus antibodies of the present invention, which were isolated from the DP32 family, bind to proteins having sulfated tyrosine epitopes. Such proteins include, but are not limited to, PSGL-1, GPIb, α-2-antiplasmin; aminopeptidase B; CC chemokine receptors such as CCR2, CCR5, CCR3, CXCR3, CXCR4, CCR8, and CCR2b; seven-transmembrane-segment (7TMS) receptors; coagulation factors such as factor V, VIII, and IX; fibrinogen gamma chain; heparin cofactor II; secretogranins such as secretogranin I and II; vitronectin, amyloid precursor, α-2-antiplasmin; cholecystokinin; α-choriogonadotropin; complement C4; dermatan sulfateproteoglycan; fibronectin; and castrin. In a preferred embodiment, the consensus antibody of the present invention binds to sulfated CC chemokine receptors such as CCR5, CXCR4, and CCR2b. Sulfated tyrosines may contribute to the binding of CCR5 to MIP-1α, MIPβ, and HIV-1 gp120/CD4 and to the ability of HIV-1 to enter cells expressing CCR5 and CD4.

Moreover, the binding of the antibodies of the present invention may be dependent on the stage of development of the cell (AML subtype is classified based on the French-American-British system using the morphology observed under routine processing and cytochemical staining). The antibodies may bind to AML cells that are of subtype M3 or above, but not M0 or M1 subtype cells. In addition, the antibodies may or may not bind M2 subtype cells. Accordingly, the antibodies of the present invention show low binding to normal, healthy bone marrow (e.g., CD34+ cells). It is thought that such differences are based on alterations in PSGL-1 expression and/or sulfation, as well as possible conformational changes in PSGL-1 that expose a slightly different epitope.

Therefore, the consensus antibody may not bind undifferentiated cells in the bone marrow such as M₀, M₁, M₂, and M₃ cells. It is believed that PSGL-1, to which the consensus antibody binds, is not expressed in significant levels or is not sulfated on these undifferentiated cells. The consensus antibody of the present invention may also not bind to healthy bone marrow cells (such as CD34+ cells).

In a more preferred embodiment, the consensus antibody of the present invention binds to sulfated PSGL-1. The S15 antibody, in particular, exhibits enhanced selectivity for sulfated PSGL-1. White cells involved in inflammation, such as monocytes, neutrophils, and lymphocytes, are primarily recruited by the four adhesion molecules, PSGL-1, P-selectin, VLA-4, and VCAM-1 in the inflammatory processes of diseases such as atherosclerosis (Huo and Ley, Acta Physiol. Scand., 173: 35-43 (2001); Libby, Sci. Am. May: 48-55 (2002); Wang et al., J. Am. Coll. Cardiol. 38: 577-582 (2001)). The interference of the consensus antibody, and particularly S15, with any of these central molecules suggests a potential role for the consensus antibody in abrogating related diseases.

Specifically, P-selectin controls cell attachment and rolling. Additionally, P-selectin-PSGL-1 interactions activate a number of other molecules on cells which are integrally connected with tumorigenesis (when concerned with malignant cells) and inflammatory responses (when concerned with white blood cells) (Shebuski and Kilgore, J. Pharmacol. Exp. Ther. 300: 729-735 (2002)). Based on this understanding of P-selectin's ability to regulate cellular processes, it is apparent that the enhanced scFv selectivity of the consensus antibody and S15 for sulfated PSGL-1 may make them a superior molecule for treating a variety of malignant and inflammatory diseases. Moreover, models of malignant disease have shown that P-selectin binding to malignant cells requires sulfation of PSGL-1 (Ma and Geng, J. Immunol. 168: 1690-1696 (2002)). This requirement is similar to that for binding of the consensus antibody and particularly for S15 binding. Thus, one can expect that the consensus antibody and particularly S15 could abrogate P-selectin facilitation of progressing malignant disease.

The consensus antibody of the present invention, particularly in embodiments where X₂ and X₃ are arginine, and X₆ is isoleucine, and preferably A3R, also exhibits enhanced selectivity for sulfated GPIb. GPIb is involved in aggregation of platelets involved by high shear in regions of arterial stenosis and platelet activation induced by low concentrations of thrombin. Based on this understanding of GPIb, it is apparent that the enhanced scFv selectivity of the consensus antibody and A3R for sulfated GPIb may make it a superior molecule for treating a variety of cardiovascular and inflammatory diseases.

Preferably, the consensus antibody of the present invention binds to an epitope present on at least one cell type involved in inflammation or tumorogenesis, including T-ALL cells, AML cells, Pre-B-ALL cells, B-leukemia cells, B-CLL cells, multiple myeloma cells, and metastatic cells. Further preferably, the consensus antibody of the present invention may bind to epitopes on a lipid, carbohydrate, peptide, glycolipid, glycoprotein, lipoprotein, and/or lipopolysaccharide molecule. Such epitopes preferably have at least one sulfated moiety. Alternatively, but also preferably, the consensus antibody of the present invention cross-reacts with two or more epitopes, each epitope having one or more sulfated tyrosine residues, and at least one cluster of two or more acidic amino acids, an example of which is PSGL-1. These antibodies or fragments thereof of the present invention may be internalized into the AML cells, for example, following binding to PSGL-1. Such internalization may occur via endocytosis and an active process that is process, time and temperature dependent.

It is the hypervariable regions of the consensus antibody and the antibodies that bind with substantially the same affinity as S15, A3R, S1, S11, D1 and D3 according to the present invention that participate in forming the antigen binding sites. The antigen-binding site is complementary to the structure of the epitopes to which the antibodies bind, therefore these binding sites are referred to as complementarity-determining regions (CDRs). There are three CDRs on each light and heavy chain of an antibody (CDR), CDR2, and CDR3), each located on the loops that connect the β strands of the V_{H} and V_{L} domains. The most variable of these regions is the CDR3 region of the heavy chain. The CDR3 region is understood to be the most exposed region of the Ig molecule and, as provided herein, has a central role in determining the selective and/or specific binding characteristics observed.

DP32, which is one of the 49 germ lines present in the phage display library, is the specific germ line of the phage library from which the consensus antibody of the present invention was isolated. Therefore, DP32 provides the antibodies of the present invention with at least the heavy and light chain framework variable regions, light chain CDR, CDR2, and CDR3 regions, and/or heavy chain CDR1 and CDR2. DP32 also provides a three-dimensional structure on which the hypervariable regions were conformed. It is well known that the specificity of an antibody is determined by its three-dimensional conformation. Thus, the limitations imposed by DP32 may have a significant role in determining the specificity of the antibodies of the present invention. Moreover, DP32 has various charged amino acids, which may have a structural role in the antibodies' antigen recognition.

According to the present invention, CDRs may also be inserted into cassettes to produce antibodies. A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends. The amino acid sequence of the cassette may ostensibly be fixed, whereas the replaced, inserted, or attached sequence can be highly variable. The cassette can be comprised of several domains, each of which encompasses a function crucial to the final construct. The cassette of a particular embodiment of the present invention comprises, from the N-terminus, framework region 1 (FR1), CDR1, framework region 2 (FR2), CDR2, framework region 3 (FR3), and framework region 4 (FR4).

In an embodiment of the invention, it is possible to replace distinct regions within the cassette. For example, the CDR2 and CDR1 hypervariable regions of the cassette may be replaced or modified by non-conservative or, preferably, conservative amino acid substitutions.

In the present invention, the consensus antibody and the antibodies that bind with substantially the same affinity as A3R and S15, have a heavy and a light chain, and each chain has a first, second, and third hypervariable region, which are the CDR3, CDR2, and CDR1 regions, respectively. The binding selectivity and specificity are determined particularly by the CDR3 region of a chain, possibly by the CDR3 region of the light chain and, preferably, by the CDR3 region of the heavy chain, and secondarily by the CDR2 and CDR1 regions of the light chain and, preferably, of the heavy chain. The binding selectivity and specificity may also be secondarily influenced by the upstream or downstream regions flanking the first, second, and/or third hypervariable regions.

Preferably, the consensus sequence of the consensus antibody is within the hypervariable regions of the consensus antibody. In particular, the consensus sequence may be in the CDR3 region, the CDR2 region, or the CDR1 region of the consensus antibody. All of or only a portion of the consensus sequence may be in the CDR3 region, the CDR2 region, or the CDR1 region. For example, the consensus sequence may overlap two hypervariable regions or may be partially within one or more hypervariable regions and partially within another part of the variable region of the consensus antibody. Preferably, the consensus sequence is in the CDR3 region. Also preferably, the consensus sequence excludes a CDR3 region comprising the amino acid sequence of SEQ ID NO:4.

In particular, in one embodiment, the consensus antibody preferably includes one or more amino acid sequences of SEQ ID NO:9, SEQ ID NO:17, and SEQ ID NO:18. In an alternative embodiment, the consensus sequence preferably includes one or more amino acid sequences of SEQ ID NO:10, SEQ ID NO:17, and SEQ ID NO:18. The amino acid sequences are preferably within the hypervariable regions of the consensus antibody. In particular, the amino acid sequences may be in the CDR3 region, the CDR2 region, or the CDR1 region of the consensus antibody. All or only a portion of the amino acid sequences may be in the CDR3 region, the CDR2 region, or the CDR1 region. Preferably the amino acid sequence of SEQ ID NO:9 or SEQ ID NO:10 is in the CDR3 region, the amino acid sequence of SEQ ID NO:17 is in the CDR2 region, and the amino acid sequence of SEQ ID NO:18 is in the CDR1 region of the consensus antibody.

The present invention also provides antibodies that bind to sulfated PSGL-1 and/or sulfated GPIb with substantially the same affinity as S15, A3R, S1, S11, D1 and/or D3. In one embodiment, the antibodies bind to sulfated PSGL-1 and/or sulfated GPIb with substantially the same affinity as S11 and in this embodiment the antibody preferably comprise one or more of amino acid sequence of SEQ ID NO:9, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:9 is in the CDR3 region, the amino acid sequence of SEQ ID NO:17 is in the CDR2 region, and the amino acid sequence of SEQ ID NO:18 is in the CDR1 region of the antibody.

In another embodiment, the antibodies of the present invention bind to sulfated PSGL-1 and/or sulfated GPIb with substantially the same affinity as A3R and in this embodiment the antibody preferably comprise one or more of amino acid sequence of SEQ ID NO:10, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:10 is in the CDR3 region, the amino acid sequence of SEQ ID NO:17 is in the CDR2 region, and the amino acid sequence of SEQ ID NO:18 is in the CDR1 region of the antibody.

In another embodiment, the antibodies of the present invention bind to sulfated PSGL-1 and/or sulfated GPIb with substantially the same affinity as S1 and in this embodiment the antibody preferably comprise one or more of amino acid sequence of SEQ ID NO:28, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:28 is in the CDR3 region, the amino acid sequence of SEQ ID NO:17 is in the CDR2 region, and the amino acid sequence of SEQ ID NO:18 is in the CDR1 region of the antibody.

In another embodiment, the antibodies of the present invention bind to sulfated PSGL-1 and/or sulfated GPIb with substantially the same affinity as S11 and in this embodiment the antibody preferably comprise one or more of amino acid sequence of SEQ ID NO:31, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:31 is in the CDR3 region, the amino acid sequence of SEQ ID NO:17 is in the CDR2 region, and the amino acid sequence of SEQ ID NO:18 is in the CDR1 region of the antibody.

In another embodiment, the antibodies of the present invention bind to sulfated PSGL-1 and/or sulfated GPIb with substantially the same affinity as D1 and in this embodiment the antibody preferably comprise one or more of amino acid sequence of SEQ ID NO:14, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:14 is in the CDR3 region, the amino acid sequence of SEQ ID NO:17 is in the CDR2 region, and the amino acid sequence of SEQ ID NO:18 is in the CDR1 region of the antibody.

In another embodiment, the antibodies of the present invention bind to sulfated PSGL-1 and/or sulfated GPIb with substantially the same affinity as D3 and in this embodiment the antibody preferably comprise one or more of amino acid sequence of SEQ ID NO:13, SEQ ID NO:17, and SEQ ID NO:18. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:13 is in the CDR3 region, the amino acid sequence of SEQ ID NO:17 is in the CDR2 region, and the amino acid sequence of SEQ ID NO:18 is in the CDR1 region of the antibody.

In another embodiment, antibodies or fragments of antibodies, of the present invention bind to cells expressing the chemokine receptor CCR5 and/or cells expressing the chemokine receptor CXCR4 with substantially the same or better affinity as S11 scFv. In this embodiment, the antibody or preferably comprise one or more of amino acid sequence of SEQ ID NO:17; SEQ ID NO:18, and SEQ ID NO:31. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:31 is in the CDR3 region, the amino acid sequence of SEQ ID NO:17 is in the CDR2 region, and the amino acid sequence of SEQ ID NO:18 is in the CDR1 region of the antibody.

For all of the amino acid sequences of ≦25 amino acid residues described and detailed herein (e.g., CDRs, CDR flanking regions), it is to be understood and considered as a further embodiment of the invention that these amino acid sequences include within their scope one or two amino acid substitution(s) and that preferably the substitutions are conservative amino acid substitutions. For all of the amino acid sequences of >25 amino acid residues described and detailed herein, it is to be understood and considered as an embodiment of the invention that these amino acid sequences include within their scope an amino acid sequence with ≧90% sequence similarity to the original sequence (Altschul et al., Nucleic Acids Res. 25: 3389-402 (1997)). Similar or homologous amino acids are defined as non-identical amino acids which display similar properties, e.g., acidic, basic, aromatic, size, positively or negatively charged, polarity, non-polarity.

Percent amino acid similarity or homology or sequence similarity is determined by comparing the amino acid sequences of two different peptides or polypeptides. Antibody sequences were determined by DNA sequencing. The two sequences are aligned, usually by use of one of a variety of computer programs designed for the purpose, and amino acid residues at each position are compared. Amino acid identity or homology is then determined. An algorithm is then applied to determine the percentage amino acid similarity. It is generally preferable to compare amino acid sequences, due to the greatly increased sensitivity to detection of subtle relationships between the peptide, polypeptide or protein molecules. Protein comparison can take into account the presence of conservative amino acid substitutions, whereby a mismatch may yet yield a positive score if the non-identical amino acid has similar physical and/or chemical properties (Altschul et al. (1997), supra).

In an embodiment of the invention, the three hypervariable regions of each of the light and heavy chains can be interchanged between the two chains and among the three-hypervariable sites within and/or between chains.

According to the present invention, the consensus antibodies and the antibodies that bind with substantially the same affinity as S15, A3R and/or D1/D3 include IgG, IgA, IgD, IgE, or IgM antibodies. The IgG class encompasses several sub-classes including IgG₁, IgG₂, IgG₃, and IgG₄.

Antibodies may be provided in many forms, such as fragments, complexes, and multimers. According to the present invention, antibody fragments include Fv, scFv, dsFv, Fab, Fab₂, and Fd molecules. Smaller antibody fragments, such as fragments of Fvs and fragments of Fabs, are also included in the term “fragments”, as long as they retain the binding characteristics of the original antibody or larger fragment. Examples of such fragments would be (1) a minibody, which comprises a fragment of the heavy chain only of the Fv, (2) a microbody, which comprises a small fractional unit of antibody heavy chain variable region (International Application No. PCT/IL99/00581), (3) similar bodies having a fragment of the light chain, and (4) similar bodies having a functional unit of a light chain variable region. Constructs include, for example, multimers such as diabodies, triabodies, and tetrabodies. The term “antibody” is intended to encompass all of these molecules, as well as derivatives, combinations, modifications, homologs, mimetics, and variants thereof, unless it is specified otherwise or indicated otherwise based on context and/or knowledge in the art.

It has been established that scFv penetrate tissues and are cleared from the blood more rapidly than a full size antibody because they are smaller in size (Adams et al., Br. J. Cancer 77: 1405-12 (1988); Hudson, Curr. Opin. Immunol. 11(5): 548-557 (1999); Wu et al., Tumor Targeting 4: 47 (1999)). Thus, scFv are often employed in diagnostics, prognostics, or staging involving radioactive labels such as tumor imaging to allow for a more rapid clearance of the radioactive label from the body. A number of cancer targeting scFv multimers have recently undergone pre-clinical evaluation for in vivo stability and efficacy (Adams et al. (1988), supra; Wu (1999), supra).

Typically, scFv monomers are designed with the C-terminal end of the V_{H} domain tethered by a polypeptide linker to the N-terminal residue of the V_{L}. Optionally an inverse orientation is employed: the C-terminal end of the V_{L} domain is tethered to the N-terminal residue of V_{H} through a polypeptide linker (Power et al., J. Immun. Meth. 242: 193-204 (2000)). The polypeptide linker is typically around fifteen amino acids in length.

When the linker is reduced to about three to seven amino acids, the scFvs can not fold into a functional Fv domain and instead associate with a second scFv to form a diabody. Further reducing the length of the linker to less than three amino acids forces the scFv association into trimers or tetramers, depending on the linker length, composition and Fv domain orientations. (Powers (2000), supra).

Recently, it has been discovered that multivalent antibody fragments such as scFv dimers, trimers, and tetramers often provide higher affinity over the binding of the parent antibody to the target. This higher Unity offers potential advantages including improved pharmaco-kinetics for tumor targeting applications. Additionally, in studying P-Selectin and its ligand PSGL-1, which are involved in tethering and rolling of leukocytes, scientists have concluded that cells expressing dimeric forms of PSGL-1 established more stable rolling adhesions because of this higher binding affinity. These adhesions are more sheer resistant and exhibited less fluctuation in rolling velocities. (Ramachandran et al., PNAS, 98(18) 10166-71 (2001)).

The greater binding affinity of these multivalent forms may be beneficial in diagnostics and therapeutic regimens. For example, a scFv may be employed as a blocking agent to bind a target receptor and thus block the binding of the “natural” ligand. In such instances, it is desirable to have a higher affinity association between the scFv and the receptor to decrease chances for disassociation, which may allow an undesirable binding of the natural ligand to the target. In addition, this higher affinity may be useful when the target receptors are involved in adhesion and rolling or when the target receptors are on cells present in areas of high sheer flow, such as platelets.

Once an antibody having desired binding capabilities has been selected and/or developed, it is well within the ability of one skilled in the art using the guidance provided herein to produce constructs and fragments which retain the characteristics of the original antibody. For example, fill antibody molecules, Fv fragments, Fab fragments, Fab₂ fragments, dimers, trimers, and other constructs can be made which retain the desired characteristics of the originally selected or developed antibody.

If it is desired to substitute amino acids, but still retain the characteristics of an antibody, it is well within the skill in the art to make conservative amino acid substitutions. Modifications such as conjugating to various agents may also be made to antibodies without altering their binding characteristics. Other modifications, such as those made to produce more stable antibodies may also be made to antibodies or fragments without altering their specificity. For example, peptoid modification, semipeptoid modification, cyclic peptide modification, N terminus modification, C terminus modification, peptide bond modification, backbone modification, and residue modification may be performed. It is also within the ability of the skilled worker following the guidance of the present specification to test the modified antibodies or fragments to assess whether their binding characteristics have been changed.

Likewise, it is within the ability of the skilled worker using the guidance provided herein to alter the binding characteristics of an antibody to obtain a molecule with more desirable characteristics. For example, once an antibody having desirable properties is identified, random or directed mutagenesis may be used to generate variants of the antibody, and those variants may be screened for desirable characteristics.

Using conventional methods known in the art, one of skill would also be able to determine additional antibodies that have the binding capabilities of the consensus antibody and/or that bind specifically to sulfated PSGL-1 or sulfated GPIb and bind with affinity substantially similar to that of the S15 and A3R. For example, additional antibodies such as D1 and D3, can be isolated using the biopanning methods described herein, wherein the molecule or cell that the consensus antibody binds is used to screen a particular phage display library, particularly a library prepared from a leukemia, lymphoma, and myeloma patient.

Antibodies according to the present invention, may also have a tag that may be inserted or attached thereto to aid in the preparation and identification thereof, and in diagnostics. The tag can later be removed from the molecule. Examples of useful tags include: AU1, AU5, BTag, c-myc, FLAG, Glu-Glu, HA, His6, HSV, HTTPHH, IRS, KT3, Protein C, S-TAG®, T7, V5, and VSV-G (Jarvik and Telmer, Ann. Rev. Gen., 32, 601-18 (1998)). The tag is preferably c-myc or KAK.

The present invention provides for scFv antibodies. As used herein, a scFv is defined as a molecule which is made up of a variable region of a heavy chain of a human antibody and a variable region of a light chain of a human antibody, which may be the same or different, and in which the variable region of the heavy chain is connected, linked, fused, or covalently attached to, or associated with, the variable region of the light chain.

A scFv construct may be a multimer (e.g., dimer, trimer, tetramer, and the like) of scFv molecules that incorporate one or more of the hypervariable domains of the antibody. All scFv derived constructs and fragments retain enhanced binding characteristics so as to bind selectively and/or specifically to a target cell in favor of other cells. The binding selectivity and/or specificity is primarily determined by hypervariable regions. The antibodies of the subject invention can be constructed to fold into multivalent Fv forms, which may improve binding affinity and specificity and increased half-life in blood.

Mulitvalent forms of scFv have been designed and produced by others. One approach has been to link two scFvs with linkers. Another approach involves using disulfide bonds between two scFvs for the linkage. The simplest approach to production of dimeric or trimeric Fv was reported by Holliger et al., PNAS 90: 6444-48 (1993) and Kortt et al., Protein Eng. 10: 423-33 (1997). One such method was designed to make dimers of scFvs by adding a sequence of the FOS and JUN protein region to form a leucine zipper between them at the c-terminus of the scFv (Kostelny et al., J Immunol. 148(5): 1547-53 (1992); De Kruif et al., J Biol Chem. 271(13): 7630-34 (1996)). Another method was designed to make tetramers by adding a streptavidin coding sequence at the c-terminus of the scFv. Streptavidin is composed of 4 subunits, so when the scFv-streptavidin is folded, 4 subunits accommodate themselves to form a tetramer (Kipriyanov et al., Hum Antibodies Hybridomas 6(3): 93-101 (1995)). In yet another method, to make dimers, trimers, and tetramers, a free cysteine is introduced in the protein of interest. A peptide-based cross linker with variable numbers (2 to 4) of maleimide groups was used to cross link the protein of interest to the free cysteines (Cochran et al., Immunity 12(3): 241-50 (2000)).

In this system, the phage library (as described herein above) can be designed to display scFvs, which can fold into the monovalent form of the Fv region of an antibody. Further, and also discussed herein above, the construct is suitable for bacterial expression. The genetically engineered scFvs comprise heavy chain and light chain variable regions joined by a contiguously encoded 15 amino acid flexible peptide spacer. The preferred spacer is (Gly₄Ser)₃ (SEQ ID NO:8). The length of this spacer, along with its amino acid, constituents provides for a nonbulky spacer, which allows the V_{H} and the V_{L} regions to fold into a functional Fv domain that provides effective binding to its target.

Varying the length of the spacers is yet another preferred method of forming dimers, trimers, and triamers (often referred to in the art as diabodies, triabodies, and tetrabodies, respectively). Dimers are formed under conditions where the spacer joining the two variable chains of a scFv is shortened to generally 5-12 amino acid residues. This shortened spacer prevents the two variable chains from the same molecule from folding into a functional Fv domain. Instead, the domains are forced to pair with complimentary domains of another molecule to create two binding domains. In a preferred method, a spacer of only 5 amino acids (Gly₄Ser) (SEQ ID NO:19) was used for diabody construction. This dimer can be formed from two identical scFvs, or from two different populations of scFvs and retain the selective and/or specific enhanced binding activity of the parent scFv(s), and/or show increased binding strength or affinity.

In a similar fashion, triabodies are formed under conditions where the spacer joining the two variable chains of a scFv is shortened to generally less than 5 amino acid residues, preventing the two variable chains from the same molecule from folding into a functional Fv domain instead, three separate scFv molecules associate to form a trimer. In a preferred method, triabodies were obtained by completely removing this flexible spacer. The triabody can be formed from three identical scFvs, or from two or three different populations of scFvs, and retain the selective and/or specific enhanced binding activity of the parent scFv(s), and/or show increased binding strength or affinity.

Tetrabodies are similarly formed under conditions where the spacer joining the two variable chains of a scFv is shortened to generally less than 5 amino acid residues, preventing the two variable chains from the same molecule from folding into a functional Fv domain. Instead, four separate scFv molecules associate to form a tetramer. The tetrabody can be formed from four identical scFvs, or from 1-4 individual units from different populations of scFvs and should retain the selective and/or specific enhanced binding activity of the parent scFv(s), and/or show increased binding strength or affinity. Whether triabodies or tetrabodies form, under conditions where the spacer is generally less than 5 amino acid residues long, depends on the amino acid sequence of the particular scFv(s) in the mixture and the reaction conditions.

The present invention also provides polypeptides comprising a consensus sequence: X₁-X₂-X₃-Pro-X₅-X₆ (SEQ ID NO:3), wherein X₁ and X₆ are hydrophobic amino acids and X₂, X₃, and X₅ are any amino acid. In one embodiment, of the polypeptide, X₂ is selected from the group consisting of arginine, lysine, and X₁ and X₆ are selected from the group consisting of leucine, valine, methionine, alanine, phenylalanine, and isoleucine. The polypeptide may preferably comprise SEQ ID. NO:5. Alternatively, the polypeptide may comprise SEQ ID NO:6. In another embodiment of the polypeptide according to the present invention, X₂ and X₃ are arginine and the hydrophobic amino acid X₆ is preferably isoleucine. In another embodiment of the polypeptide according to the present invention, X₁ is selected from the group consisting of leucine and methionine, X₂ and X₃ are arginine, X₅ is selected from the group consisting of serine and valine and X₆ is isoleucine. The polypeptides of the present invention may be substantially circular or looped.

The present invention also provides polypeptides that bind specifically to PSGL-1, wherein the polypeptide binds with an affinity substantially similar to S15. Alternatively or in addition, the polypeptides can bind specifically to GPIb with an affinity substantially similar to A3R.

The present invention also provides polypeptides that bind to CCR5 and/or CXCR4, wherein the polypeptide binds with an affinity substantially similar or better than S11. Alternatively or in addition, the polypeptides can bind specifically to cells expressing CCR5 and/or CXCR4 with an affinity substantially similar or better than S11.

The present invention further provides isolated or purified polypeptides, such as recombinant nucleic acids, that encode the antibodies and polypeptides of the present invention. Such isolated or purified polypeptides can be produced in prokaryotic or eukaryotic expression systems. Such expression systems include expression vectors and host cells transfected with such expression vectors. Methods for producing antibodies and polypeptides in prokaryotic and eukaryotic systems, including culturing recombinant host cells under conditions permitting expression of such antibodies and isolating or purifying such antibodies from the recombinant host cells or from culture medium are well-known in the art.

An eukaryotic cell system, as defined in the present invention and as discussed, refers to an expression system for producing peptides or polypeptides by genetic engineering methods, wherein the host cell is an eukaryote. An eukaryotic expression system may be a mammalian system, and the peptide or polypeptide produced in the mammalian expression system, after purification, is preferably substantially free of mammalian contaminants. Other examples of a useful eukaryotic expression system include yeast expression systems.

A preferred prokaryotic system for production of the peptide or polypeptide of the invention uses E. coli as the host for the expression vector. The peptide or polypeptide produced in the E. coli system, after purification, is substantially free of E. coli contaminating proteins. Use of a prokaryotic expression system may result in the addition of a methionine residue to the N-terminus of some or all of the sequences provided for in the present invention. Removal of the N-terminal methionine residue, after peptide or polypeptide production to allow for full expression of the peptide or polypeptide, can be performed as is known in the art, one example being with the use of Aeromonas aminopeptidase under suitable conditions (U.S. Pat. No. 5,763,215).

The present invention also provides a process for selecting entities, e.g., antibodies or fragments thereof or alternatively small inorganic chemical entities, that bind sulfated epitopes. These methods involve panning a library (e.g., a phage display library to identify antibodies or fragments thereof and combinatorial libraries to identify small inorganic chemical entities) against a peptide having a sulfated epitope. Suitable sulfated epitopes for panning may be based on or derived from, for example, PSGL-1, GPIb, α-2-antiplasmin; aminopeptidase B; CC chemokine receptors such as CCR2, CCR5, CCR3, CXCR3, CXCR4, CCR8, and CCR2b; seven-transmembrane-segment (7TMS) receptors; coagulation factors such as factor V, VIII, and IX; fibrinogen gamma chain; heparin cofactor II; secretogranins such as secretogranin I and II; vitronectin, amyloid precursor, α-2-antiplasmin; cholecystokinin; α-choriogonadotropin; complement C4; dermatan sulfateproteoglycan; fibronectin; or castrin. Such peptides can be sulfated at any position. Preferably, the peptide comprises the sulfated epitope is derived from or based on a region of PSGL-1 (especially when sulfated at the tyrosine residue at position 51 from the N-terminus), GPIb (especially when sulfated at the tyrosine residue at position 276 and to a lesser extend the tyrosine residue at position 279), or CCR5 (especially when sulfated at the tyrosine residue at position 10). In one embodiment, the method comprises immobilizing the peptide on a solid support. Optionally, the method comprises competitive panning using a non-sulfated, soluble peptide or a soluble peptide sulfated at an alternate tyrosine position. Panning an appropriate combinatorial library to identify a small inorganic chemical entity can, of course, also be used to carry out these methods.

In a preferred embodiment of the present invention, the process for producing an entity that binds sulfated epitopes (e.g., an antibody or polypeptide of the present invention) comprises the steps of: (a) providing a library (e.g., phage display library); (b) providing a peptide of PSGL-1 of SEQ ID NO:7; (c) panning the library to select for an entity (e.g., phage particle) that binds to the immobilized peptide of SEQ ID NO:7; and (d) producing the selected entity (e.g., antibody or polypeptide comprising the scFv antibody) that binds to the peptide of SEQ ID NO:7.

In a preferred embodiment of the present invention, the process for producing an entity (e.g., antibody or polypeptide of the present invention) comprises the steps of: (a) providing a library (e.g., phage display library); (b) providing an immobilized peptide of PSGL-1 (SEQ ID NO:7); (c) panning the library to select for an entity (e.g., phage particle) that binds to the immobilized peptide of SEQ ID NO:7 in the presence of a soluble unsulfated PSGL-1 peptide (SEQ ID NO:26); and (d) producing the entity (e.g., antibody or polypeptide comprising the scFv antibody) that binds to the peptide of SEQ ID NO:7.

In a preferred embodiment of the present invention, the process for producing an entity (e.g., antibody and polypeptide of the present invention) comprises the steps of: (a) providing a library (e.g., phage display library); (b) providing an immobilized peptide of PSGL-1 (SEQ ID NO:7); (c) panning the library for an entity (e.g., phage particle) that binds to the immobilized peptide of SEQ ID NO:7 in the presence of a soluble sulfated GPIb peptide (SEQ ID NO:44 and/or 50); and (d) producing the entity (e.g., antibody or polypeptide comprising the scFv antibody) that binds to the peptide of SEQ ID NO:7. In a preferred embodiment of the present invention, the process for producing an entity (e.g., antibody or polypeptide of the present invention) comprises the steps of: (a) providing a library (e.g., phage display library); (b) providing an immobilized peptide of PSGL-1 (SEQ ID NO:7); (c) panning the library for an entity (e.g., phage particle) that binds to the immobilized peptide of SEQ ID NO:7 in the presence of a soluble sulfated GPIb peptide (SEQ ID NO:44 and/or 50) or unsulfated PSGL-1 peptide (SEQ ID NO:26); and (d) producing the entity (e.g., antibody or polypeptide comprising the scFv antibody) that binds to the peptide of SEQ ID NO:7.

In a preferred embodiment of the present invention, the process for producing an entity (e.g., antibody or polypeptide of the present invention) comprises the steps of: (a) providing a library (e.g., phage display library); (b) providing an immobilized peptide of PSGL-1 (SEQ ID NO:7); (c) panning the library for an entity (e.g., phage particle) that binds to the immobilized peptide of SEQ ID NO:7 in the presence of a soluble sulfated GPIb peptide (SEQ ID NO:44 and/or 50) and soluble unsulfated GPIb peptide (SEQ ID NO:43); and (d) producing the entity (e.g., antibody or polypeptide comprising the scFv antibody) that binds to the peptide of SEQ ID NO:7.

In a preferred embodiment of the present invention, the process for producing an entity (e.g., antibody or polypeptide of the present invention) comprises the steps of: (a) providing a library (e.g., phage display library); (b) providing an immobilized peptide of PSGL-1 (SEQ ID NO:7); (c) panning the library for an entity (e.g., phage particle) that binds to the immobilized peptide of SEQ ID NO:7 in the presence of a soluble sulfated GPIb peptide (selected from SEQ ID NO:44, 50, 57 and 58 or combination thereof) and/or unsulfated PSGL-1 peptide (SEQ ID NO:43); and (d) producing the entity (e.g., antibody or polypeptide comprising the scFv antibody) that binds to the peptide of SEQ ID NO:7. In another embodiment of the present invention, the process for producing an entity (e.g., antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 and (d) producing the entity (e.g., antibody or polypeptide comprising the scFv antibody) that binds to the peptide of SEQ ID NO:44.

In another embodiment of the present invention, the process for producing an entity (e.g. antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 in the presence of a soluble unsulfated GPIb peptide (SEQ ID NO:43); and (d) producing the entity (e.g., antibody or polypeptide comprising the scFv antibody) that binds to the peptide of SEQ ID NO:44.

In another embodiment of the present invention, the process for producing an entity (e.g., antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 in the presence of a one or more of the soluble sulfated PSGL-1 peptides of SEQ ID NO:7, 48, 49 or 59; and (d) producing the entity (e.g., antibody or polypeptide comprising the scFv antibody) that binds to the peptide of SEQ ID NO:44.

In another embodiment of the present invention, the process for producing an entity (e.g., antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 in the presence of a one or more of the soluble sulfated PSGL-1 peptides of SEQ ID NO:7, 48, 49 or 59 and soluble unsulfated PSGL-1 peptide (SEQ ID NO:26); and (d) producing the entity (e.g., antibody or polypeptide comprising the scFv antibody) that binds to the peptide of SEQ ID NO:44.

In another embodiment of the present invention, the process for producing an entity (e.g., antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 in the presence of a one or more of the soluble sulfated PSGL-1 peptides of SEQ ID NO:7, 48, 49 or 59 and soluble unsulfated GPIb peptide (SEQ ID NO:43); and (d) producing the entity (e.g., antibody or polypeptide comprising the scFv antibody) that binds to the peptide of SEQ ID NO:44.

In another embodiment of the present invention, the process for producing an entity (e.g., antibody or polypeptide of the present invention) comprises the steps of: (a) providing a library (e.g., phage display library); (b) providing an immobilized peptide of GPIb peptide (SEQ ID NO:44); (c) panning the library for an entity (e.g., phage particle) that binds to the immobilized peptide of SEQ ID NO:44 in the presence of a one or more of the soluble sulfated PSGL-1 peptides of SEQ ID NO:7, 48, 49 or 59 and one or more of the soluble sulfated GPIb peptides (SEQ ID NO:44 and/or 50); and (d) producing the entity (e.g., antibody or polypeptide comprising the scFv antibody) that binds to the peptide of SEQ ID NO:44.

In another embodiment of the present invention, the process for producing an entity (e.g., antibody or polypeptide of the present invention) comprises the steps of: (a) providing a library (e.g., phage display library); (b) providing an immobilized peptide of CCR5 (SEQ ID NO:53); (c) panning the library for an entity (e.g., phage particle) that binds to the immobilized peptide of SEQ ID NO:53 and (d) producing the entity (e.g., antibody or polypeptide comprising the scFv antibody) that binds to the peptide of SEQ ID NO:53.

In another embodiment of the present invention, the process for producing an entity (e.g., antibody or polypeptide of the present invention) comprises the steps of: (a) providing a library (e.g., phage display library); (b) providing an immobilized peptide of CCR5 (SEQ ID NO:53); (c) panning the library for an entity (e.g., phage particle) that binds to the immobilized peptide of SEQ ID NO: 53 in the presence of a soluble unsulfated GPIb peptide (SEQ ID NO:43) and/or unsulfated soluble PSGL-1 peptide (SEQ ID NO:26) (d) producing the entity (e.g., antibody or polypeptide comprising the scFv antibody) that binds to the peptide of SEQ ID NO:53.

In another embodiment of the present invention, the process for producing an entity (e.g., antibody or polypeptide of the present invention) comprises the steps of: (a) providing a library (e.g., phage display library); (b) providing an immobilized peptide of CCR5 (SEQ ID NO:53); (c) panning the library for an entity (e.g., phage particle) that binds to the immobilized peptide of SEQ ID NO: 53 in the presence of a soluble sulfated GPIb peptide (SEQ ID NO:44 and/or 50) and/or soluble sulfated PSGL-1 peptides (SEQ ID NO:7, 48, 49 and/or 59) and (d) producing the entity (e.g., antibody or polypeptide comprising the scFv antibody) that binds to the peptide of SEQ ID NO:53.

In another embodiment of the present invention, the process for producing an entity (e.g., antibody or polypeptide of the present invention) comprises the steps of: (a) providing a library (e.g., phage display library); (b) providing an immobilized peptide of CCR5 (SEQ ID NO:53); (c) panning the library for an entity (e.g., phage particle) that binds to the immobilized peptide of SEQ ID NO: 53 in the presence of a soluble sulfated GPIb peptide (SEQ ID NO:44 and/or 50) and/or unsulfated soluble PSGL-1 peptide (SEQ ID NO:26); and (d) producing the entity (e.g., antibody or polypeptide comprising the scFv antibody) that binds to the peptide of SEQ ID NO:53.

In another embodiment of the present invention, the process for producing an entity (e.g., antibody or polypeptide of the present invention) comprises the steps of: (a) providing a library (e.g., phage display library); (b) providing an immobilized peptide of CCR5 (SEQ ID NO:53); (c) panning the library for an entity (e.g., phage particle) that binds to the immobilized peptide of SEQ ID NO: 53 in the presence of a soluble sulfated GPIb peptide (SEQ ID NO:44 and/or 50) and/or unsulfated soluble GPIb peptide (SEQ ID NO:43); and (d) producing the entity (e.g., antibody or polypeptide comprising the scFv antibody) that binds to the peptide of SEQ ID NO:53.

In the alternative approach for therapeutic targeting of sulfated tyrosine epitopes present on proteins, such as GPIb and PSGL-1, a small inorganic chemical entity may be identified by screening of an appropriate combinatorial library. Such a chemical entity may have a number of advantages over a scFv or IgG-based therapeutic agent. For example, an inorganic chemical entity may be administered orally and have an enhanced biosafety profile, including reduced immuno-crossreactivity. It may provide enhanced selectivity towards the target, particularly following rational drug design to optimize an initially selected lead compound. Other advantages include lower production costs, longer shelf-life and a less complicated regulatory approval process.

Since a number of embodiments of the epitope of the invention have been identified, e.g. on GPIb and PSGL-1, a ligand-driven approach may be taken to identify inorganic chemical entities, which have very narrow specificity, or alternately, target more than one sulfated tyrosine epitope for disease states such as re-perfusion injury which involves more than one distinct target each bearing such an epitope. The ligand-driven approach significantly shortens the screening process for identifying targets for therapeutic intervention, and enables simultaneous target validation with lead optimization, which may be carried out with a series of focused libraries.

A library of inorganic chemical entities specialized for targeting sulfated tyrosine epitopes may be designed and developed first by analyzing the three dimensional interaction between an antibody such as Y1 and its known targets such as residues sulfated Tyr-276 and Asp-277 of GPIb. Chemical libraries composed of entities that mimic the Y1 binding site and which provide increased affinity to the target may be developed by computer assisted combinatorial library design.

The present invention also provides a library for identifying human antibodies that bind to sulfated epitopes. The library is of immunoglobulin binding domains comprising a diverse antigen-binding domain for complementary binding, wherein the library has diversity only in heavy chain CDR3. Preferably, the immunoglobulin binding domains are scFv molecules. Also preferably, the immunoglobulin binding domains have heavy chain complementarity determining regions (CDRs) 1 and 2 derived from DP32 and, more preferably, also have light chain variable regions derived from DP32. The immunoglobulin binding domains of the present library can be displayed on the surface of any suitable vector, such as filamentous bacteriophage particles, for example. In one embodiment, the libraries of the present invention can be used to select for sulfated motifs or epitopes.

The antibodies and binding fragments thereof of the subject invention can be associated with, combined, fused, or linked to various agents, such as drugs, toxins, pharmaceuticals, and radioactive isotopes with, optionally, a pharmaceutically effective carrier, to form drug-peptide compositions, fusions or conjugates having anti-disease and/or anti-cancer activity. Such conjugates and fusions may also be used for diagnostic, prognostic, or staging purposes.

Examples of carriers useful in the invention include dextran, HPMA (a hydrophilic polymer), or any other polymer, such as a hydrophilic polymer, as well as derivatives, combinations and modifications thereof. Alternatively, decorated liposomes can be used, such as liposomes decorated with scFv Y1 molecules, such as Doxil, a commercially available liposome containing large amounts of doxorubicin. Such liposomes can be prepared to contain one or more desired agents and be admixed with the antibodies of the present invention to provide a high drug to antibody ratio.

Alternatively, the link between the antibody or polypeptide and the agent may be a direct link. A direct link between two or more neighboring molecules may be produced via a chemical bond between elements or groups of elements in the molecules. The chemical bond can be, for example, an ionic bond, a covalent bond, a hydrophobic bond, a hydrophilic bond, an electrostatic bond, or a hydrogen bond. The bonds can be, for example, amide, carbon-sulfide, peptide, and/or disulfide bonds. In order to attach the antibody to the agent or linker, amine, carboxy, hydroxyl, thiol and ester functional groups may be used, as is known in the art to form covalent bonds.

The link between the peptide and the agent or between the peptide and carrier, or between the carrier and agent may be via a linker compound. As used herein, a linker compound is defined as a compound that joins two or more moieties. The linker can be straight-chained or branched. A branched linker compound may be composed of a double-branch, triple branch, or quadruple or more branched compound. Linker compounds useful in the present invention include those selected from the group having dicarboxylic acids, malemido hydrazides, PDPH, carboxylic acid hydrazides, and small peptides.

More specific examples of linker compounds useful, according to the present invention, include: (a) dicarboxylic acids such as succinic acid, glutaric acid, and adipic acid; (b) maleimido hydrazides such as N-[maleimidocaproic acid] hydrazide, 4-[N-maleimidomethyl]cyclohexan-1-carboxylhydrazide, and N-[maleimidoundecanoic acid] hydrazide; (c) (3-[2-pyridyldithio]propionyl hydrazide); and (d) carboxylic acid hydrazides selected from 2-5 carbon atoms, and derivatives, combinations, modifications, and analogues thereof.

Linking via direct coupling using small peptide linkers is also useful. For example, direct coupling between the free sugar of, for example, the anti-cancer drug doxorubicin and a scFv may be accomplished using small peptides. Examples of small peptides include AU1, AU5, BTag, c-myc, FLAG, Glu-Glu, HA, His6, HSV, HTTPHH, IRS, KT3, Protein C, S-TAG®, T7, V5, VSV-G, and KAK.

Antibodies and polypeptides of the present invention may be bound to, conjugated to, complexed with, or otherwise associated with imaging agents (also called indicative markers), such as radioisotopes, and these conjugates can be used for diagnostic, prognostic, or staging and imaging purposes. Kits having such radioisotope-antibody (or fragment) conjugates are provided.

Examples of radioisotopes useful for diagnostics, prognostics, staging and imaging include ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}telluriunm ¹⁶⁵thulium, ₁₆₇thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium, and ⁶³gallium. Preferred radioactive isotopes, are opaque to X-rays or any suitable paramagnetic ions.

The indicative marker molecule may also be a fluorescent marker molecule. Examples of fluorescent marker molecules include fluorescein, phycoerythrin, or rhodamine, or modifications or conjugates thereof.

Antibodies and polypeptides conjugated to indicative markers may be used to diagnose, prognoses or stage disease states. Moreover, the present invention also provides a method of purging tumor cells from a patient by providing a sample containing cells from the patient and incubating the cells from the patient with an antibody of the present invention. Such activities may be carried out in vivo, in vitro, or ex vivo. Where the diagnosis, prognosis, or staging is carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

The present invention thus provides for a diagnostic kit for in vitro analysis of treatment efficacy before, during, or after treatment, having an imaging agent having a peptide of the invention linked to an indicative marker molecule, or imaging agent. The invention further provides for a method of using the imaging agent for diagnostic localization and imaging of a cancer, more specifically a tumor, having the following steps: (a) contacting the cells with the composition; (b) measuring the radioactivity bound to the cells; and hence (c) visualizing the tumor.

Examples of suitable imaging agents include fluorescent dyes, such as FITC, PE, and the like, and fluorescent proteins, such as green fluorescent proteins. Other examples include radioactive molecules and enzymes that react with a substrate to produce a recognizable change, such as a color change.

In one example, the imaging agent of the kit is a fluorescent dye, such as FITC, and the kit provides for analysis of treatment efficacy of cancers, more specifically blood-related cancers, e.g., leukemia, lymphoma, and myeloma. FACS analysis is used to determine the percentage of cells stained by the imaging agent and the intensity of staining at each stage of the disease, e.g., upon diagnosis, during treatment, during remission and during relapse.

The present invention also provides a method of diagnosing, prognosing, or staging a disease in a patient by providing a sample containing a cell from the patient and determining whether the antibodies of the present invention bind to the cell of the patient, thereby indicating that the patient is at risk for or has the disease. Such activities may be carried out in vivo, in vitro, or ex vivo. Where carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

With respect to cancer, staging a disease in a patient generally involves determining the classification of the disease based on the size, type, location, and invasiveness of the tumor. One classification system to classify cancer by tumor characteristics is the “TNM Classification of Malignant Tumours” (6th Edition) (L. H. Sobin, Ed.), which is incorporated by reference herein and which classifies stages of cancer into T, N, and M categories with T describing the primary tumor according to its size and location, N describing the regional lymph nodes, and M describing distant metastases. In addition, the numbers I, II, III and IV are used to denote the stages and each number refers to a possible combination of TNM factors. For example, a Stage I breast cancer is defined by the TMN group: T1, N0, M0 which mean: T1—Tumor is 2 cm or less in diameter, N0—No regional lymph node metastasis, M0—No distant metastasis. Another system is used to stage AML, with subtypes of classified based on the French-American-British system using the morphology observed under routine processing and cytochemical staining.

In addition, a recently proposed World Health Organization (WHO) staging or classification of neoplastic diseases of the hematopoietic and lymphoid tissues includes (specifically for AMLs) traditional FAB-type categories of disease, as well as additional disease types that correlate with specific cytogenetic findings and AML associated with myelodysplasia. Others have also proposed pathologic classifications. For example, one proposal specific for AML includes disease types that correlate with specific cytogenetic translocations and can be recognized reliably by morphologic evaluation and immunophenotyping and that incorporate the importance of associated myelodysplastic changes. This system would be supported by cytogenetic or molecular genetic studies and could be expanded as new recognizable clinicopathologic entities are described (Arber, Am. J. Clin. Pathol. 115(4): 552-60 (2001)).

Antibodies and polypeptides of the present invention may be bound to, conjugated to, or otherwise associated with anti-cancer agents, anti-neoplastic agents, anti-viral agents, anti-metastatic agents, anti-inflammatory agents, anti-thrombosis agents, anti-restenosis agents, anti-aggregation agents, anti-autoimmune agents, anti-adhesion agents, anti-cardiovascular disease agents, pharmaceutical agents, or other anti-disease agents. An agent refers to an agent that is useful in the prophylactic treatment or diagnosis of a mammal including, but not restricted to, a human, bovine, equine, porcine, murine, canine, feline, or any other warm-blooded animal.

Examples of such agents include, but are not limited to, anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid, and derivatives, combinations and modifications thereof.

Exemplary pharmaceutical agents include anthracyclines such as doxorubicin (adriamycin), daunorubicin, idarubicin, detorubicin, caminomycin, epirubicin, esorubicin, morpholinodoxorubicin, morpholinodaunorubicin, methoxymorpholinyldoxorubicin, methoxymorpholinodaunorubicin and methoxymorpholinyldoxorubicin and substituted derivatives, combinations and modifications thereof. Further exemplary pharmaceutical agents include cis-platinum, taxol, calicheamicin, vincristine, cytarabine (Ara-C), cyclophosphamide, prednisone, fludarabine, idarubicin, chlorambucil, interferon alpha, hydroxyurea, temozolomide, thalidomide and bleomycin, and derivatives, combinations and modifications thereof.

Inhibition of growth of a cancer cell includes, for example, the (i) prevention of cancerous or metastatic growth, (ii) slowing down of the cancerous or metastatic growth, (iii) the total prevention of the growth process of the cancer cell or the metastatic process, while leaving the cell intact and alive, (iv) interfering contact of cancer cells with the microenvironment, or (v) killing the cancer cell.

Inhibition of growth of a leukemia cell includes, for example, the (i) prevention of leukemic or metastatic growth, (ii) slowing down of the leukemic or metastatic growth, (iii) the total prevention of the growth process of the leukemia cell or the metastatic process, while leaving the cell intact and alive, (iv) interfering contact of cancer cells with the microenvironment, or (v) killing the leukemia cell.

In one embodiment, the present invention provides methods of inducing or activating ADCC by administering the present antibodies. Accordingly, the consensus antibody and the antibodies that bind with substantially the same affinity as S15 and A3R of the present invention may activate ADCC and/or stimulate natural killer (NK) cells (e.g. CD56+), γδ T cells, and/or monocytes, which may result in cell lysis. Generally, following administration of an antibody comprising an Fc region or portion of the antibody, the antibody binds to an Fc receptor (FcR) on effector cells, for example, NK cells, triggering the release of perforin and granzyme B and/or induction of FasL expression, which then leads to apoptosis.

Binding of FasL expressed on effector cells to the Fas receptor on the target cell surface may induce target cell apoptosis via activation of the Fas receptor signal transduction pathway. In one embodiment, an IgG antibody comprising the consensus sequence of the invention induces FasL expression on effector cells. Various factors can affect ADCC, including the type of effector cells involved, cytokines (IL-2 and G-CSF, for example), incubation time, the number of receptors present on the surface of the cells, and antibody affinity.

In one embodiment, the present invention provides methods of inhibiting entry of the HIV virus into cells, the method comprising providing an antibody or fragment thereof of the present invention to cells that express either CCR5 and/or CXCR4. As described above, for HIV entry into cells, it is believed that it must first interact with CD4 and then with a co-receptor (e.g. CCR5 or CXCR4) to allow entry of the HIV into the cell. The gp120 envelope glycoprotein binds to CD4, which in turn induces conformational changes in gp120 that create or expose a binding site for a co-receptor. The binding of the gp120/CD4 complex to the co-receptor then drives additional conformational changes that eventually lead to the insertion of the gp41 fusion protein into the host cell membrane, thus provoking fusion and entry. Kwong, et al., J. Virol. 74: 1961-72 (2000). It is believed that when an antibody or fragment thereof, of the present invention binds to the co-receptor, it decreases or inhibits the ability of the co-receptor to associate with gp120 and thus inhibits the necessary conformational changes that are believed to be necessary for entry of the gp41 fusion peptide into the host cell membrane.

In another embodiment, antibodies or fragments of antibodies, of the present invention bind to cells expressing the chemokine receptor CCR5 and/or cells expressing the chemokine receptor CXCR4 with substantially the same or better affinity as S11 scFv. In this embodiment, the antibody or preferably comprise one or more of amino acid sequence of SEQ ID NO:17; SEQ ID NO:18, and SEQ ID NO:31. Preferably these amino acid sequences are in the hypervariable region of the antibody. In particular, the amino acid sequences may be in the CDR3 region, the CDR2 region, or the CDR1 region of the antibody. All or only a portion of the amino acid sequences may be in the CDR3 region, the CDR2 region, or the CDR1 region. Preferably the amino acid sequence of SEQ ID NO:31 is in the CDR3 region, the amino acid sequence of SEQ ID NO:17 is in the CDR2 region, and the amino acid sequence of SEQ ID NO:18 is in the CDR1 region of the antibody.

Examples of anti-disease, anti-cancer, and anti-leukemic agents to which antibodies and polypeptides of the present invention may usefully be linked include toxins, radioisotopes, and pharmaceuticals.

Examples of toxins include gelonin, Pseudomonas exotoxin (PE), PE40, PE38, diphtheria toxin, ricin, or derivatives, combinations and modifications thereof. Examples of radioisotopes include gamma-emitters, positron-emitters, and x-ray emitters that may be used for localization and/or therapy, and beta-emitters and alpha-emitters that may be used for therapy. The radioisotopes described previously as useful for diagnostics are also useful for therapeutics.

Non-limiting examples of anti-cancer or anti-leukemia agents include anthracyclines such as doxorubicin (adriamycin), daunorubicin, idarubicin, detorubicin, caminomycin, epirubicin, esorubicin, morpholinodoxorubicin, morpholinodaunorubicin, methoxymorpholinyldoxorubicin, methoxymorpholinodaunorubicin and methoxymorpholinyldoxorubicin and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof.

In one embodiment, the pharmaceutical compositions of the present invention have an antibody or polypeptide comprising any of the consensus sequences of the present invention and a pharmaceutically acceptable carrier. The antibody or polypeptide can be present in an amount effective to inhibit cell rolling, inflammation, auto-immune disease, metastasis, growth and/or replication of tumor cells or leukemia cells, or increase in number of tumor cells in a patient having a tumor or leukemia cells in a patient having leukemia. Alternatively, the antibody or polypeptide can be present in an amount effective to increase mortality of tumor cells or leukemia cells. Also alternatively, the antibody or polypeptide can be present in an amount effective to alter the susceptibility of diseased cells to damage by anti-disease agents, tumor cells to damage by anti-cancer agents, or leukemia cells to damage by anti-leukemia agents. Further alternatively, the antibody or polypeptide can be present in an amount effective to decrease number of tumor cells in a patient having a tumor or leukemia cells in a patient having leukemia. Yet further alternatively, the antibody or polypeptide can be present in an amount effective to inhibit restenosis in which case the consensus antibody preferably comprises A3R. The antibody, or polypeptide can also be present in an amount effective to inhibit HIV entry and/or treat HIV infection. Alternatively, the antibody or polypeptide, can be used as a targeting agent to direct a therapeutic to a specific cell or site.

Antibodies and polypeptides of the present invention may be administered to patients in need thereof via any suitable method. Exemplary methods include intravenous, intramuscular, subcutaneous, topical, intratracheal, intrathecal, intraperitoneal, intralymphatic, nasal, sublingual, oral, rectal, vaginal, respiratory, buccal, intradermal, transdermal, or intrapleural administration.

For intravenous administration, the formulation preferably will be prepared so that the amount administered to the patient will be an effective amount from about 0.1 mg to about 1000 mg of the desired composition. More preferably, the amount administered will be in the range of about 1 mg to about 500 mg of the desired composition. The compositions of the invention are effective over a wide dosage range and depend on factors such as the particulars of the disease to be treated, the half-life of the peptide, or polypeptide-based pharmaceutical composition in the body of the patient, physical and chemical characteristics of any agent complexed with antibody or fragment thereof and of the pharmaceutical composition, mode of administration of the pharmaceutical composition, particulars of the patient to be treated or diagnosed, as well as other parameters deemed important by the treating physician.

Pharmaceutical composition for oral administration may be in any suitable form. Examples include tablets, liquids, emulsions, suspensions, syrups, pills, caplets, and capsules. Methods of making pharmaceutical compositions are well known in the art (See, e.g., Remington, The Science and Practice of Pharmacy, Alfonso R. Gennaro (Ed.) Lippincott, Williams & Wilkins (pub)).

The pharmaceutical composition may also be formulated so as to facilitate timed, sustained, pulsed, or continuous release. The pharmaceutical composition may also be administered in a device, such as a timed, sustained, pulsed, or continuous release device.

The pharmaceutical composition for topical administration can be in any suitable form, such as creams, ointments, lotions, patches, solutions, suspensions, lyophilizates, and gels.

Compositions having antibodies and polypeptides of the subject invention may comprise conventional pharmaceutically acceptable diluents, excipients, carriers, and the like. Tablets, pills, caplets, and capsules may include conventional excipients such as lactose, starch, and magnesium stearate. Suppositories may include excipients such as waxes and glycerol. Injectable solutions comprise sterile pyrogen-free media such as saline, and may include buffering agents, stabilizing agents or preservatives. Conventional enteric coatings may also be used.

The antibodies and polypeptides of the present invention and pharmaceutical compositions thereof, can be used in methods of treating a disease (e.g., treating can include ameliorating the effects of a disease, preventing a disease, or inhibiting the progress of a disease) in patients in need thereof. Such methods include inhibiting cell rolling, inflammation, autoimmune disease, metastasis, growth and/or replication of tumor cells or leukemia cells, or increase in number of tumor cells in a patient having a tumor or leukemia cells in a patient having leukemia. In addition, such methods include increasing the mortality rate of tumor cells or leukemia cells, alter the susceptibility of diseased cells to damage by anti-disease agents, tumor cells to damage by anti-cancer agents, or leukemia cells to damage by anti-cancer agents. Such methods also include decreasing number of tumor cells in a patient having tumor or leukemia cells in a patient having leukemia. Such methods also include inhibiting or decreasing HIV entry in cells and also, as a result of such inhibition, blocking replication of HIV and thereby treating HIV infection. Such methods further include preventing or inhibiting cardiovascular diseases such as restenosis.

The present invention moreover provides antibodies and polypeptides for use in manufacturing a medicament for the treatment of various disease states such as, e.g., AML, T-ALL, B-leukemia, B-CLL, Pre-B-ALL, multiple myeloma, metastasis, HIV infection, cardiovascular diseases, or other diseases in which such cellular functions or actions as cell rolling, inflammation, immune reactions, infection, autoimmune reactions, metastasis, play a significant role. Such medicament comprises the antibodies and the polypeptides of the present invention.

Throughout this application, reference has been made to various publications, patents, and patent applications. The teachings and disclosures of these publications, patents, and patent applications in their entireties are hereby incorporated by reference into this application to more fully describe the state of the art to which the present invention pertains.

### EXAMPLES

The following examples are set forth to aid in understanding and to further illustrate the invention, but are not intended and should not be construed to limit its scope in any way. Although specific reagents and reaction conditions are described, modifications can be made that are encompassed by the scope of the invention.

### Example 1

The present example demonstrates selection, production, and initial characterization of S15 scFv antibody fragments, including the binding capabilities of S15 antibody fragments. Briefly, a phage display library based on a specific scaffold of VH-VL with a random CDR3-VH of six amino acids was utilized to identify a scFv antibody that binds sulfated PSGl-1. The scFv antibody was obtained by panning against a synthetic sulfated peptide having the sequence of amino acids 1-17 of the mature PSGL-1 molecule from N-terminus to C-terminus or from C-terminus to N-terminus (corresponding to amino acids 42-58 of the immature PSGL-1 molecule i.e. including the signal sequence). Flow cytometry, particularly fluorescence-activated cell sorting (FACS) and ELISA was used for identifying and characterizing specific phage clones that bind to the synthetic sulfated peptide or to whole cells expressing PSGL-1.

The phage display library was constructed from a scaffold of a clone isolated from a combinatorial phage antibody library (CAT) in a pHEN vector. The scaffold contained a VH3 (1-3, 3-20) and a VL (11-7). The library was constructed by randomization of the CDR3 hypervariable loop of six amino acids in length. In order to provide a construct having a single Eag1 site at the 5′ region of the CDR3 (instead of two Eag1 sites as found in pHEN-Y1), the Eag1 restriction site at the 3′ end of the VL was mutated by excising an Xma1-Not1 fragment from the pMEN-Y1 and inserting a new fragment (by annealing an oligonucleotide of SEQ ID NO:20 and an oligonucleotide of SEQ ID NO:21) with a mutation of the ligation part of the Not1 site. The mutation was required in order to generate two unique sites (Eag1 and Xma1) and to introduce a PCR product of the same size but with randomized CDR3. After ligation, the plasmid was sequenced at the insertion region and the mutation was confirmed. The new pHEN-Y1-mut was restricted with Eag1 and Xma1 and the large fragment was the vector for subsequent ligation.

The template for the preparation of the variable CDR3 was made by PCR with an oligonucleotide of SEQ ID NO:22 and an oligonucleotide of SEQ ID NO:23. The PCR product (template A) was isolated from a SDS-polyacrylamide gel and purified for further amplifications. Template A was used for amplification with oligonucleotides of SEQ ID NO:24 and SEQ ID NO:23, the product was purified and designated template B. Template B was amplified with oligonucleotides of SEQ ID NO:25 and SEQ ID NO:23, purified, and restricted with Eag1 and Xma1 restriction enzymes. The restricted vector pHEN-Y1-mut with Eag1 and Xma1 was purified and ligated to the last PCR product restricted with the same enzymes.

The product of the ligation was used to transform TG1 cells. The yield of transformation from 3 ligations yielded 2.4×10⁶ independent colony-forming units (CFUs). The library was amplified by plating in 10 (13 cm) SOBAG (20 g Bacto-tryptone, 5 g Bacto-yeast, 8.5 mM NaCl, 10 mM MgCl₂, 0.1M glucose, 0.1 mg/ml ampicillin per liter; for plates 15 g of Bacto-agar) plates. The bacteria was resuspended in SOBAG medium from plates and kept at −70° C. in 20% glycerol. The titer of the amplified library was 1.5×10⁹ CFU/ml. An aliquot (˜10⁸ CFU) of the amplified library was infected with helper phage M13KO7 in order to rescue the library in a form of phage to carry out the biopanning experiments. This library contains on the order of 2.5×10⁶ members, as opposed to a theoretical library that contains 20⁶ or 6.4×10⁷ members.

Biopanning was carried out by incubating immobilized peptide of SEQ ID NO:7 with the phage display library, removing unbound phage by washing, and specifically eluting the bound phage. The eluted phage clones were optionally amplified before additional cycles of binding and optional amplification, enriching the pool of specific sequences in favor of those phage clones bearing antibody fragments which best bind to the peptides. After several cycles of panning, individual phage clones were characterized, and the sequences of the clones were determined.

In the present invention, the S15 antibody clone was identified by panning a phage display library in solution with streptavidin-magnetic beads bound to a biotinylated sulfated peptide of SEQ ID NO:7. This peptide was chemically synthesized and is based on the highly acidic sequence found at amino acids 42 to 58 within PSGL-1, including sulfation of the 3rd tyrosine residue. The N-terminus of the synthetic peptide was extended with aminocaproic acid and biotinylated at the amino group of the caproic acid to avoid steric hindrance of the sulfated epitope. The synthetic sulfated peptide of SEQ ID NO:7 was immobilized on streptavidin-magnetic beads (Dynal) by incubation of the peptide in excess followed by washing with PBST (PBS+0.05% Tween-20) and blocking by PBST-M (PBST supplemented with 5% low fat milk). For panning, the peptide-bound beads were incubated with 2×10¹¹ phages. Peptide of the same linear sequence (SEQ. ID. No:45) that was neither biotinylated nor sulfated was added to the panning solution in order to avoid isolating scFv clones that bind to non-sulfated peptides.

Three cycles of panning were performed using high stringency washes in PBST (20 minutes each at 37° C.). After washing, the bound phages were eluted by glycine 0.2M (pH 2.2) and neutralized by Tris 1M (pH 9.1). The eluted phage was amplified by infecting TG1 bacteria and rescued by the helper phage M13KO7. Enrichment of up to 5000 fold was achieved upon three cycles of panning. After the 3rd cycle of panning, individual clones were analyzed for amino acid sequence in the CDR3 region. The CDR3 region amino acid sequences (SEQ ID. NOS:9, 27-33, 35, 4) of those selected clones are listed in Table 1.

TABLE 1

| | |
|---|---|
| Sequence of Clones | SEQ ID NO: |

| | | | | | | | |
|---|---|---|---|---|---|---|---|
| L | R | D | P | I | M | (55%) | (SEQ ID NO:27) |
| L | R | P | P | F | L | (10%) | (SEQ ID NO:28) |
| L | R | R | P | S | I | (6%) | (SEQ ID NO:9) |
| L | T | Y | P | H | L | (6%) | (SEQ ID NO:29) |
| L | K | W | P | H | L | (3%) | (SEQ ID NO:30) |
| L | R | Y | P | F | F | (3%) | (SEQ ID NO:31) |
| L | R | S | P | V | L | (3%) | (SEQ ID NO:32) |
| V | R | H | P | I | M | (3%) | (SEQ ID NO:33) |
| L | R | H | P | V | A | (3%) | (SEQ ID NO:34) |
| M | R | A | P | V | 1 | (3%) | (SEQ ID NO:4) |

The results shown in Table 1 indicate that a strong consensus sequence was obtained: hydrophobic residues at the first and sixth positions, usually a basic residue at the second position, and a proline at the fourth position in all CDR3 sequences.

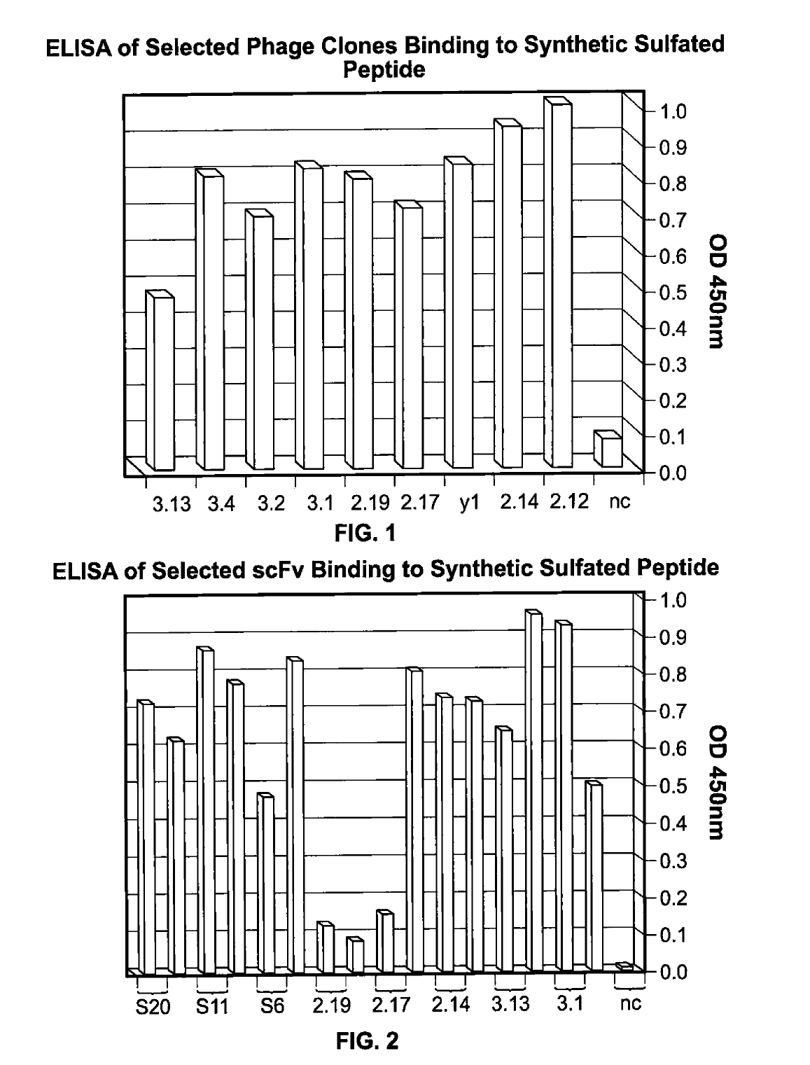

To further analyze binding to the synthetic sulfated peptide, based on a sulfated portion of PSGL-1, ELISA analyses using phage (FIG. 1) and non-purified scFv supernatants (FIG. 2) from panned and selected clones were carried out. These experiments included analyses of the scFv antibodies Y1 and L32, both previously identified and found to bind to PSGL-1. Y1 is described in U.S. patent application Ser. No. 10/029,926 and International Patent Application No. PCT/US01/49,440 and L32 is described in U.S. application Ser. No. 10/189,032). The ELISA analyses were carried out in parallel using synthetic sulfated peptide of SEQ ID NO:7 in “test” wells) and synthetic non-sulfated peptide of SEQ ID NO:26 in “background” wells. Peptides were coated on NH-CovaLink™ plates (NUNC) as recommended by the manufacturer. Binding data obtained from the background wells were subtracted from the binding data from the corresponding systems in the test wells, to generate the results shown in FIGS. 1 and 2. A randomly chosen phage clone was used as the negative control (NC) for binding to the synthetic sulfated peptide.

FIG. 1 shows that all of the panned and selected phage clones tested specifically bound to the synthetic sulfated peptide. FIG. 2 shows that all of the scFv expressed on clones selected after 3 cycles of panning specifically bound to the synthetic sulfated peptide.

Table 2 provides a summary of all the clones used in the study of Example 1 and the amino acid sequences of their CDR3 regions.

TABLE 2

| | | |
|---|---|---|
| Clone | | |
| designation | CDR3 sequence | SEQ ID NO |

| | | | | | | | |
|---|---|---|---|---|---|---|---|
| 3.1 | L | R | D | P | I | M | (SEQ ID NO:27) |
| S1 | L | R | P | P | F | L | (SEQ ID NO:28) |
| 3.13 | L | R | R | P | S | I | (SEQ ID NO:9) |
| (S15) | | | | | | | |
| S6 | L | T | Y | P | H | L | (SEQ ID NO:29) |
| S20 | L | K | W | P | H | L | (SEQ ID NO:30) |
| S11 | L | R | Y | P | F | F | (SEQ ID NO:31) |
| 3.2 | L | R | S | P | V | L | (SEQ ID NO:32) |
| 3.4 | V | R | H | P | I | M | (SEQ ID NO:33) |
| S9 | L | R | H | P | V | A | (SEQ ID NO:34) |
| Y1 | M | R | A | P | V | I | (SEQ ID NO:4) |
| 2.12 | L | R | F | P | I | A | (SEQ ID NO:36) |
| 2.14 | L | R | S | P | V | L | (SEQ ID NO:37) |
| 2.15 | L | R | S | P | P | I | (SEQ ID NO:38) |
| 2.17 | L | A | Q | P | F | G | (SEQ ID NO:39) |
| 2.18 | M | R | S | P | Y | K | (SEQ ID NO:40) |
| 2.19 | F | S | S | P | H | A | (SEQ ID NO:41) |
| NC | L | P | S | F | R | V | (SEQ ID NO:42) |

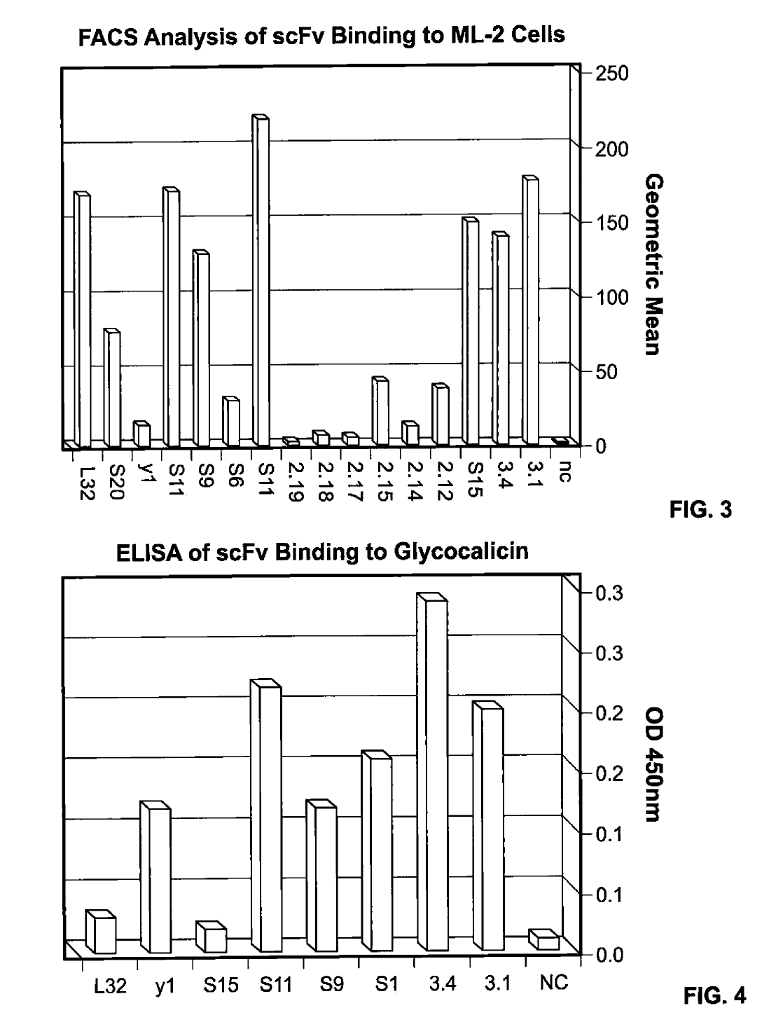

scFv from selected clones were analyzed by FACS to evaluate binding to PSGL-1 on ML-2, an AML M4 cell line which expresses PSGL-1. As shown in FIG. 3, the scFv from all of the selected clones, as well as L32 bound to PSGL-1 on ML-2 cells at different sensitivities.

ELISA analysis using non-purified scFv supernatants from panned and selected clones was carried out to analyze binding to glycocalicin, a glycoprotein expressed on platelets. The results, shown in FIG. 4., indicate that one clone, having a CDR3 sequence of SEQ ID NO:9 and designated as S15 had lower affinity to glycocalicin as compared to the other selected clones and the previously identified scFv L32.

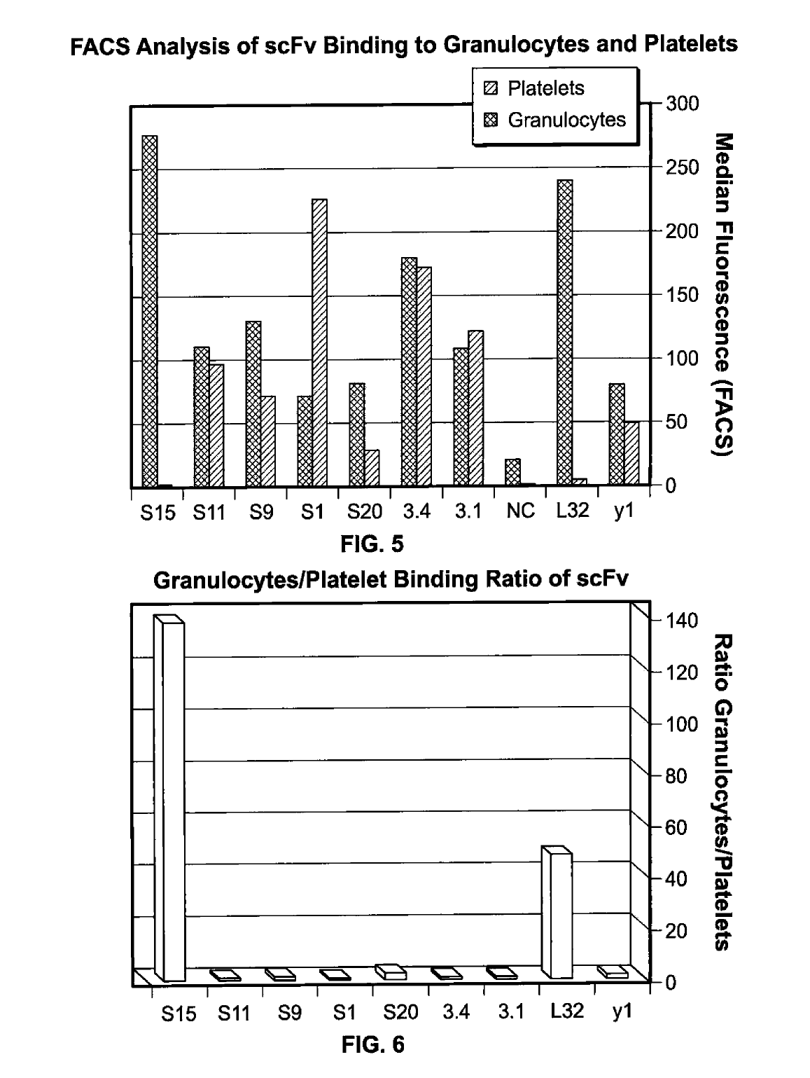

Taken together, the results shown in FIGS. 3 and 4, indicate that S15 displays high affinity for ML-2 cells that express PSGL-1 and low affinity for platelets, based on binding to glycocalicin. This conclusion was confirmed by FACS analysis of scFv binding to granulocytes (which are PSGL-1 expressing cells) and to platelets, the results of which are shown in FIG. 5. FIG. 5 clearly shows that S15 binds strongly to granulocytes, but only weakly to intact platelets.

FIG. 6 shows a comparison of the granulocyte/platelet binding ratio of scFv. Strikingly, S15 has a granulocyte/platelet binding ratio significantly higher than that of S11, S9, S1, S20, 3.4, 3.1, Y1 and L32.

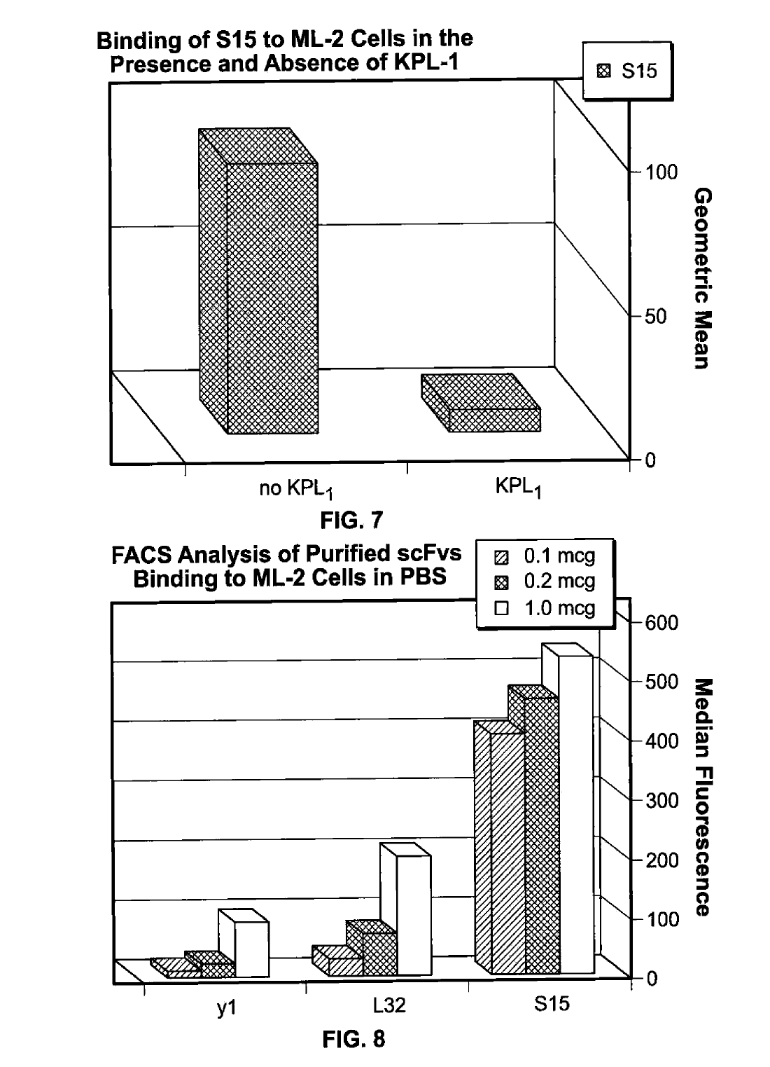

FIG. 7 shows the results of analysis of the binding of S15 to PSGL-1 on ML-2 cells in the presence and absence of KPL-1, a murine antibody directed against PSGL-1. S15 bound to ML-2 cells with high affinity in the absence of KPL-1, but such binding was essentially eliminated in the presence of KPL-1. These results confirm the specificity of S15 binding PSGL-1 cells on ML-2 cells.

S15 was purified on a Protein A affinity column for further experimentation described in Example 2.

### Example 2

The present example describes further characterization of the purified S15 scFv antibody, including its binding capabilities, compared to similarly purified L32 and Y1 scFv.

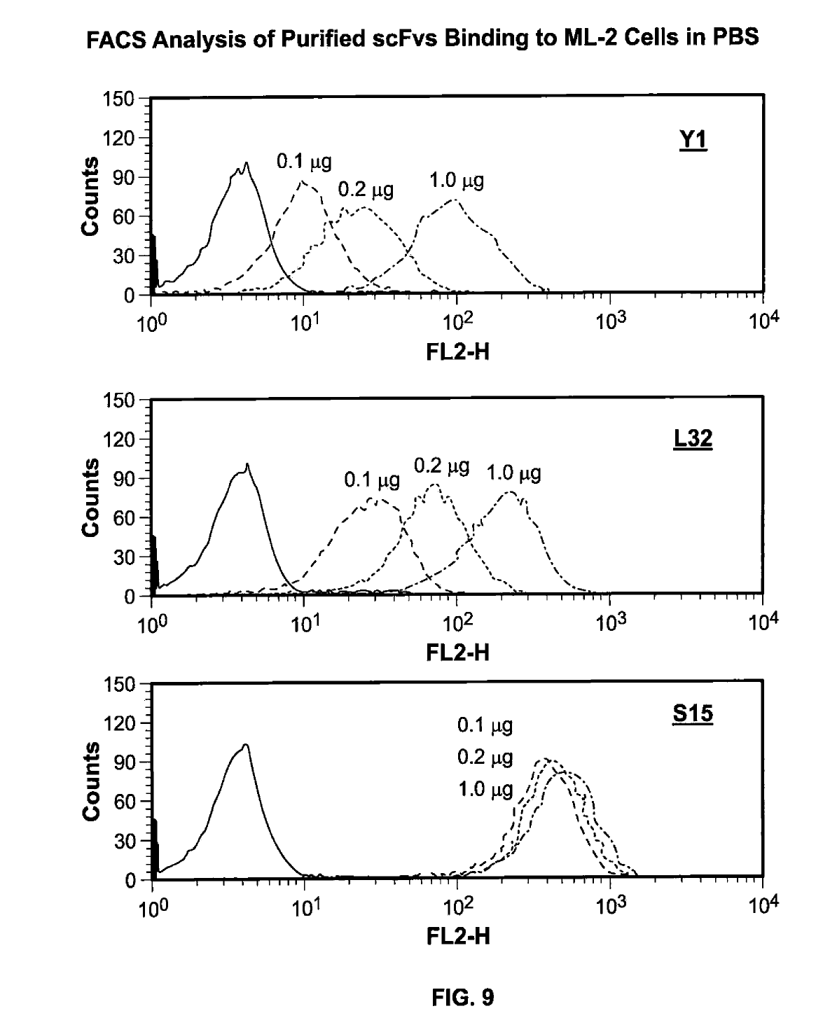

FACS analysis was carried out to analyze the binding of purified S15, Y1 and L32 scFv to ML-2 cells in the presence of PBS. The results, shown in FIGS. 8 and 9 indicate that S15 exhibits at least 100 fold greater binding to ML-2 cells as compared to L32 and Y1.

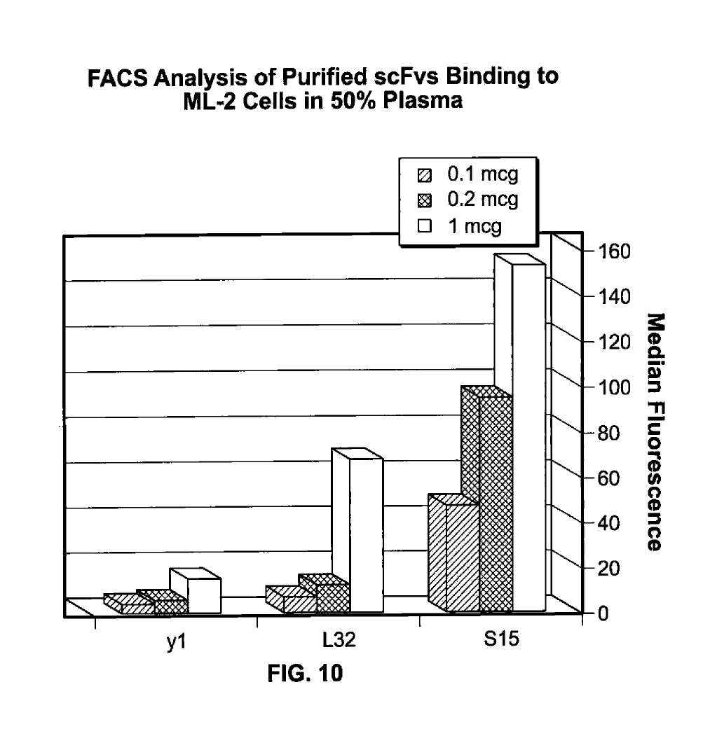

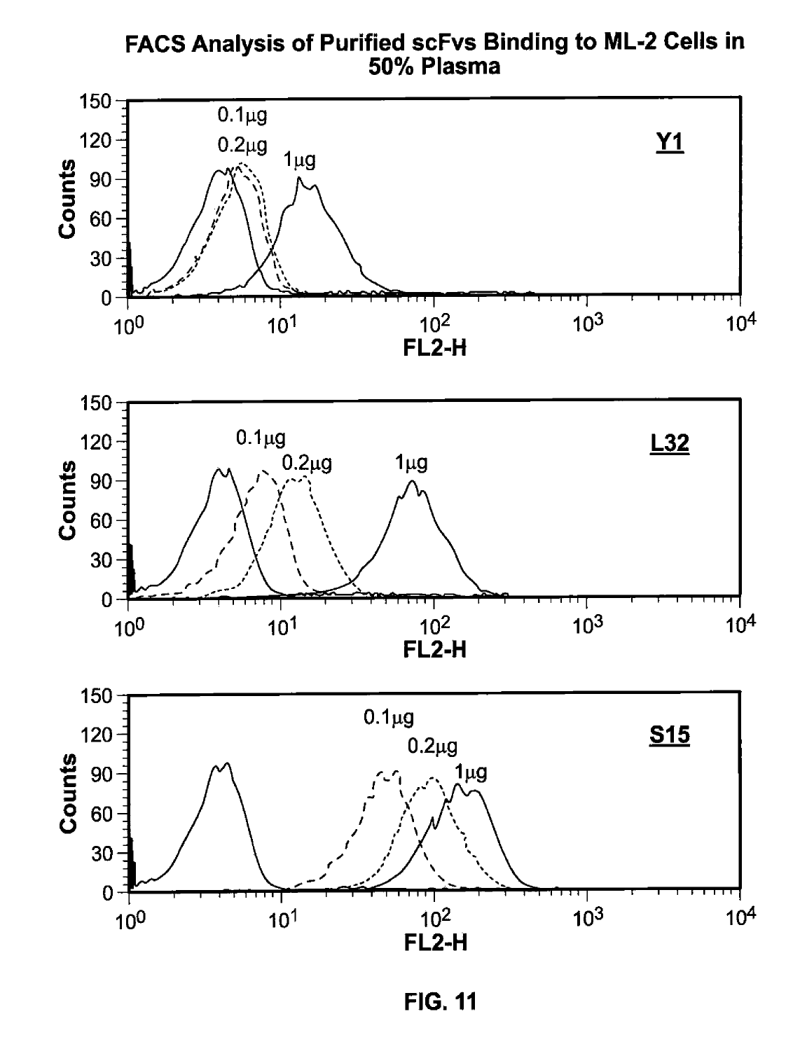

FACS analysis was carried out to analyze the binding of purified S15, Y1 and L32 scFv to ML-2 cells in the presence of 50% plasma. The results, shown in FIGS. 10 and 11, indicate that the absolute value of binding by S15 was significantly higher (by at least 10 fold) as compared to L32 and Y1.

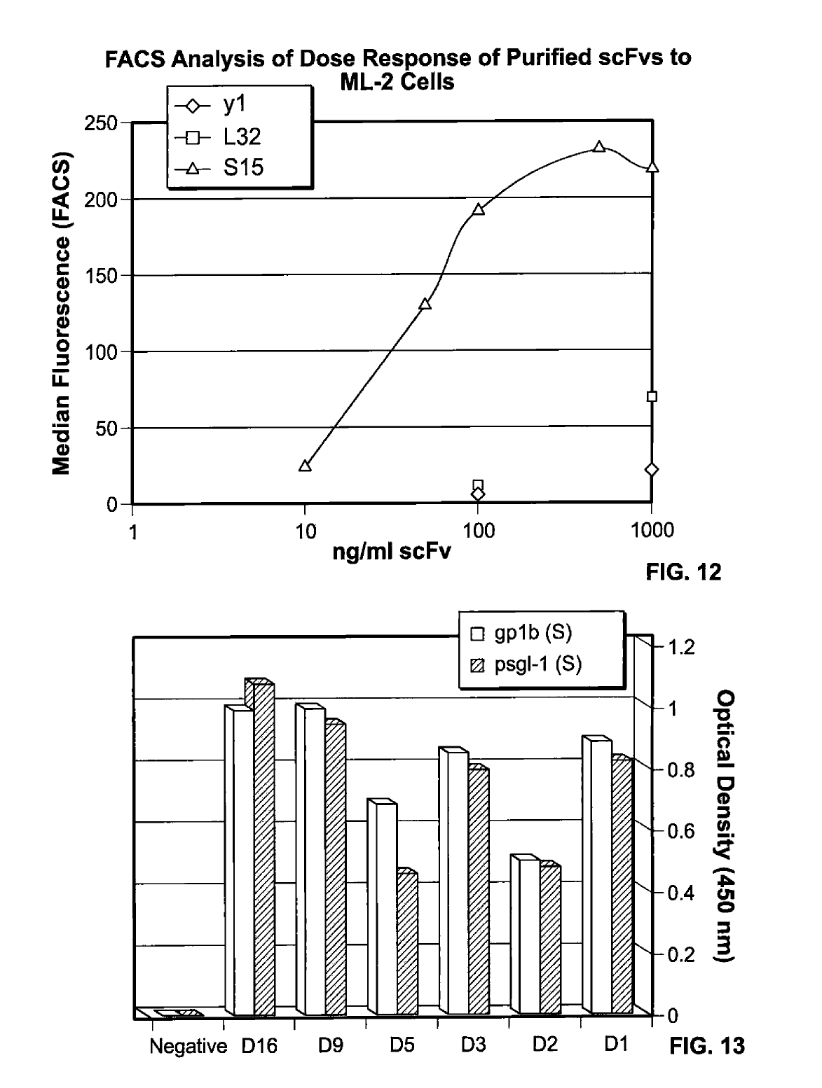

The dose response of purified S15 scFv binding to ML-2 cells was also analyzed and compared to that of L32 and Y1. As shown in FIG. 12, the dose response of S15 is significantly greater than that of L32 and Y1, even at 10 nanograms (ng).

### Example 3

This example describes the identification of two additional antibodies comprising the consensus sequence which were obtained by panning the library described in Example 1 against a synthetic peptide corresponding to residues 268-285 of GPIb including sulfation at the first tyrosine position.

The present example demonstrates selection, production, and initial characterization of D1 and D3 scFv antibody fragments, including the binding capabilities of D1 and D3 antibody fragments. Briefly, a phage display library based on a specific scaffold of VH-VL with a random CDR3-VH of six amino acids was utilized to identify an scFv antibody that binds sulfated GPIb. The scFv antibody was obtained by panning against a synthetic sulfated peptide having the sequence of amino acids 268-285 (GDEGDTDLY(SO₄)DYYPEEDTE) (SEQ ID NO:44) of the mature GPIb molecule from N-terminus to C-terminus (corresponding to amino acids 284-301 of the immature GPIb molecule, i.e., including the signal sequence). Flow cytometry, particularly fluorescence-activated cell sorting (FACS) and ELISA was used for identifying and characterizing specific phage clones that bind to the synthetic sulfated peptide or to platelets expressing GPIb.

The phage display library used is described in Example 1.

Biopanning was carried out by incubating immobilized peptide of SEQ ID NO:44 (GDEGDTDLY^{S}DYYPEEDTE) with the phage display library, removing unbound phage by washing, and specifically elating the bound phage. The elated phage clones were optionally amplified before additional cycles of binding and optional amplification, enriching the pool of specific sequences in favor of those phage clones bearing antibody fragments which best bind to the peptides. After several cycles of panning, individual phage clones were characterized, and the sequences of the clones were determined.

In the present invention, the D1 and D3 antibody clones were identified by panning a phage display library in solution with a chemically synthesized sulfated peptide of SEQ ID NO:44 covalently bound to magnetic beads. The sulfated peptide of SEQ ID NO:44 is based on the highly acidic sequence found at amino acids 268-285 within mature GPIb, including sulfation of the first tyrosine residue. The synthetic sulfated peptide of SEQ ID NO:44 was covalently bound to amine-magnetic beads (Dynal) by a reaction involving EDC/NHS, according to the manufacturer instructions. The beads were washed with PBST (PBS+0.05% Tween-20) and blocked by PBST-M (PBST supplemented with 5% low fat milk). For panning, the peptide-bound beads were incubated with 2×10¹¹ phages. Peptide of the same linear sequence (SEQ. ID. NO:43) but lacking sulfation was added to the panning solution in order to avoid isolating scFv clones that bind to non-sulfated peptides.

Three cycles of panning were performed using high stringency washes in PBST (20 minutes each at 37° C.). After washing, the bound phages were eluted by glycine 0.2M (pH 2.2) and neutralized by Tris 1M (pH 9.1). The eluted phage was amplified by infecting TG1 bacteria and rescued by the helper phage M13KO7. Enrichment of up to 5000 fold was achieved upon three cycles of panning. After the 3rd cycle of panning, individual clones were analyzed for amino acid sequence in the CDR3 region. The CDR3 region amino acid sequences (SEQ ID. NOS:11-16) of those selected clones are listed in Table 3.

TABLE 3

| | | |
|---|---|---|
| Sequence of Clones | Clone | SEQ IN NO: |

| | | | | | | | | |
|---|---|---|---|---|---|---|---|---|
| L | V | M | P | V | M | (50%) | D2 | SEQ ID NO:11 |
| L | W | Y | P | F | G | (15%) | D5 | SEQ ID NO:12 |
| L | R | V | P | F | L | (15%) | D3 | SEQ ID NO:13 |
| L | R | S | P | F | G | (10%) | D1 | SEQ ID NO:14 |
| L | S | P | P | I | F | (5%) | D9 | SEQ ID NO:15 |
| L | L | P | P | Y | G | (5%) | D16 | SEQ ID NO:16 |

The results shown in Table 3 indicate that a strong consensus sequence was obtained: hydrophobic residues at the first and sixth positions, usually a basic residue at the second position, and a proline at the fourth position in all CDR3 sequences. To further analyze binding to the synthetic sulfated peptide, based on a sulfated portion of GPIb, ELISA analyses using phage (FIG. 13) from panned and selected clones were carried out. The ELISA analyses were carried out in parallel using synthetic sulfated peptide of SEQ ID NO:44 GPIb sulfated (GDEGDTDLY^{S}DYYPEEDTE) in “test” wells and synthetic GPIb non-sulfated peptide of SEQ ID NO:43 (GDEGDTDLYDYYPEEDTE) in “background” wells as well as sulfated and non-sulfated peptides derived from PSGLl-1 respectively SEQ ID NO:7 (QATEYEYLDY^{S}DFLPETE) and SEQ ID NO:26 (QATEYEYLDYDFLPETE). Peptides were coated on NH-CovaLink™ plates (NUNC) as recommended by the manufacturer. Binding data obtained from the background wells were subtracted from the binding data from the corresponding systems in the test wells, to generate the results shown in FIG. 13. A randomly chosen phage clone was used as the negative control (NC) for binding to the synthetic sulfated peptide.

FIG. 13 shows that each of the selected phage clones specifically bound to both of the synthetic sulfated peptides i.e. GPIb and PSGL-1. While the clones exhibited a range of binding strength to the synthetic sulfated peptides, each clone displayed about the same behavior with respect to both GPIb and PSGL-1.

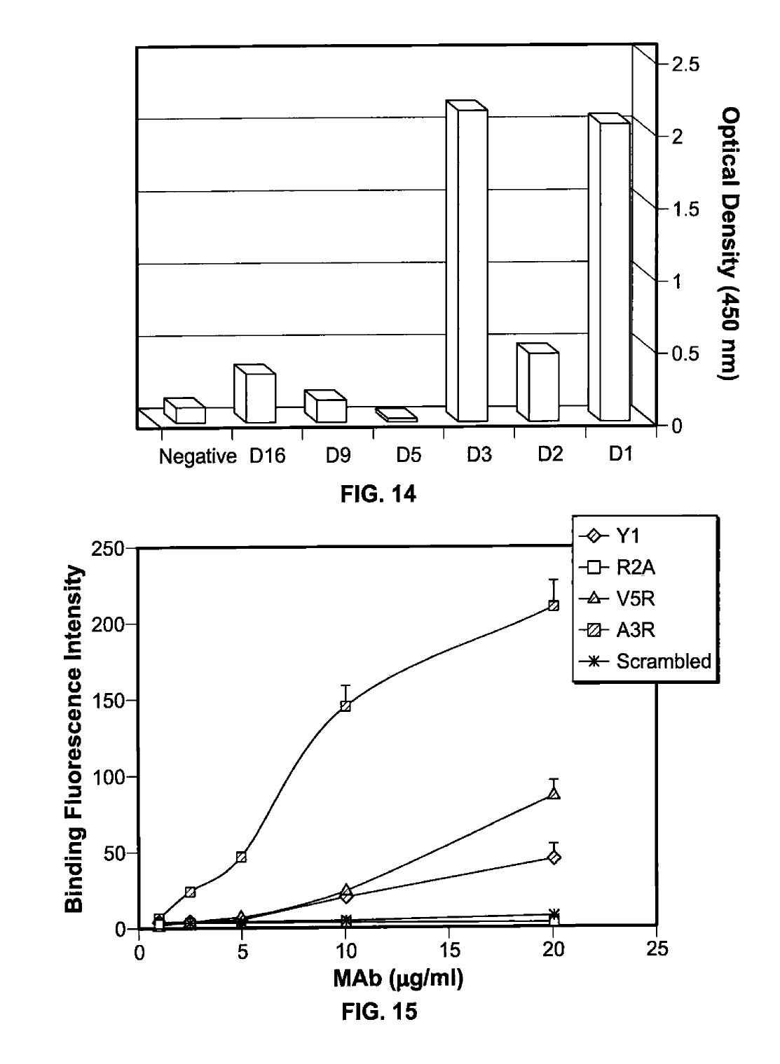

scFv from selected clones were analyzed by ELISA to evaluate binding to glycocalicin (the outer membrane portion derived from GPIb expressed in platelets). As shown in FIG. 14, scFvs D1 and D3 exhibited significant binding, while D2 and D16 exhibited weak binding and D5 and D9 were similar to the negative control.

scFvs (1 μg per experiment) from selected clones were analyzed by FACS to evaluate binding to ML-2, an AML M4 cell line expressing PSGL-1. As shown in Table 4, all of the scFvs bound to ML-2 cells, at different sensitivities. D1 and D3 exhibited the strongest binding to ML-2, presumably via interaction with an epitope of PSGL-1.

TABLE 4

| Clone | Geo Mean |
|---|---|
| Negative | 4.4 |
| D1 | 26.7 |
| D2 | 18.7 |
| D3 | 44.1 |
| D5 | 5.14 |
| D9 | 10.5 |
| D16 | 17.0 |

### Example 4

The present example demonstrates binding of several scFv antibodies having CDR3 sequences falling within the consensus sequence of the present invention. Briefly, using the CDR3 sequence of the Y1-scFv as a scaffold, mutant scFv's were produced and the effect of these mutations on the binding to platelets and granulocytes was assessed. Using site directed mutagenesis in the CDR3 of the heavy chain (CDR3H) of Y1-scFv, additional CDR3 regions falling within the consensus sequence were generated. These additional scFvs were then tested to determine the relative binding to platelets and granulocytes.

Y1-scFv is known to bind the negatively charged GPIbα epitope found on platelets. Within the six amino acid sequences comprising the CDR3H of Y1 there is an arginine residue i.e. a positively charged amino acid, in the second position. To examine the possible electrostatic effect of this arginine residue in the binding to GPIbα, four mutant scFvs were constructed. Mutant R2A has the arginine residue replaced by alanine; mutant A3R has an additional arginine at position 3, replacing alanine; mutant V5R has an additional arginine at position 5, replacing valine. The fourth scFv was a scrambled mutant. The scFv mutants are summarized in Table 5.

TABLE 5

| | | Sequence |
|---|---|---|
| | CDR3 | Identification |
| MAb | Sequence | No. |
| Y1 | MRAPVI | (SEQ ID NO:4) |
| R2A | MAAPVI | (SEQ ID NO:45) |
| A3R | MRRPVI | (SEQ ID NO:10) |
| V5R | MRAPRI | (SEQ ID NO:46) |
| scrambled | IPMARV | (SEQ ID NO:47) |

Comparative analysis of the four mutant scFvs and Y1 scFv in binding to platelets was carried out by FACS analysis, as follows.

Anti-scFv antibody was generated by immunization of a rabbit with 400 μg of a mixture of scFvs, and labeled using the Phycolink™. R-phycoerythrin conjugation kit (ProZyme, San Leandro, Calif.) according to the manufacturer's instructions. Aliquots (10 μg/mL) of scFvs were incubated with 10⁷ washed platelets for 1 hour at room temperature. The platelets were then washed in PBS containing 1% BSA and incubated with R-phycoerythrin-anti-scFv antibody for 1 hour at room temperature. Platelets were then washed, resuspended in PBS and samples (104 platelets) were analyzed by FACS (VACScan, Becton-Dickinson, CA).

FIG. 15 shows the average binding+SEM from 3 experiment using platelets from different donors. Mutant R2A does not exhibit binding to platelets, compared to the Y1 parent scFv, suggesting that the arginine in the second position of CDR3H may play a role in the platelet binding function. Mutant V5R bound to platelets in a manner similar to wild type Y1-scFv, in the concentration range between 1 to 10 μg/ml. At a concentration of 20 μg/ml however, binding of VSR was two fold higher as compared to Y1 scFv (FIG. 15). In contrast, mutant A3R scFv exhibited nine-fold higher binding to platelets compared to Y1 scFv at all the concentrations tested, suggesting that an additional arginine residue adjacent to the arginine residue at position 2 can augment the binding to platelets. The scrambled scFv failed to bind to platelets (FIG. 15), as would be expected of a mutant lacking arginine at the second position.

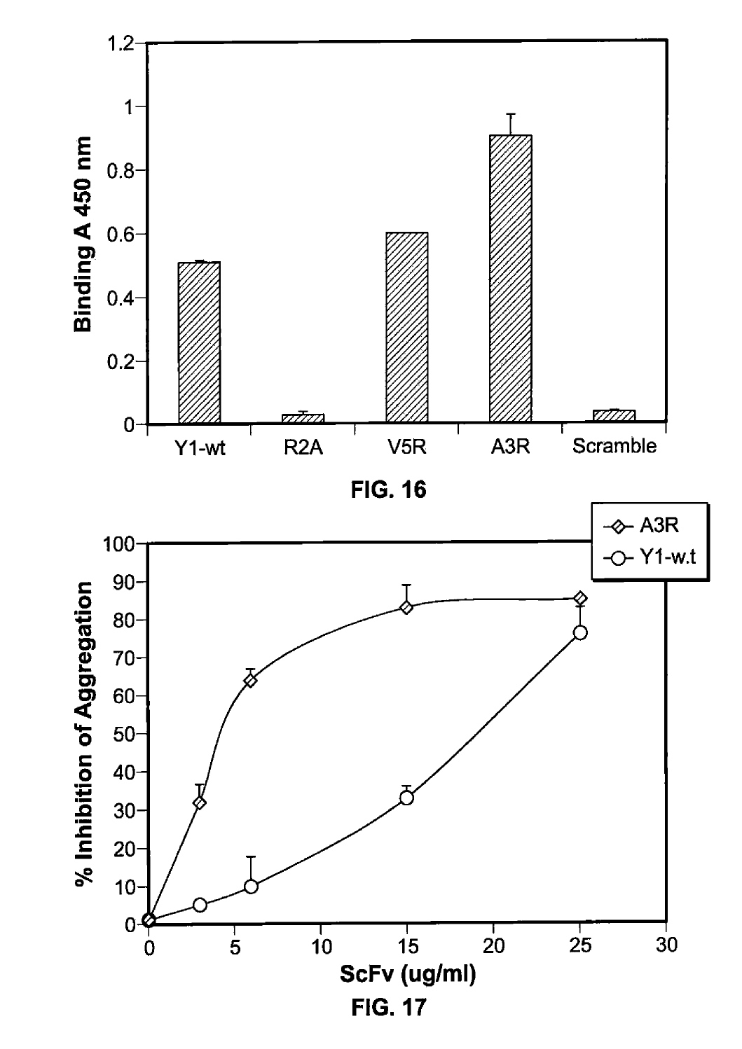

The scFv were also analyzed for their binding capacity to purified glycocalicin and to GPIbα derived peptides using ELISA assays. FIG. 16 shows these results as the average binding of 5 μg/ml scFv+STDV from 2 experiments. Mutant R2A scFv and the scrambled scFv did not bind to purified GPIbα nor to any of the GPIbα-derived peptides. V5R bound to purified glycocalicin and to GPIbα-derived peptides in a fashion similar to Y1. Mutants A3R exhibited enhanced binding to glycocalicin compared to Y1.

The effect of mutant scFv on platelet aggregation was evaluated in studies carried out as follows. Washed platelets were stirred at 500 rpm at 37° C. in a Lumiaggregometer (Chronolog, Havertown, Pa.). The difference in light transmission through the platelet suspension and suspending medium was taken as 100% aggregation. The effect of mAb-scFvs on platelet aggregation was evaluated by incubation with various concentrations of mAb-scFvs for 2 minutes at room temperature before addition of agonists and recording aggregation for four minutes.

The effect of A3R scFv on platelet aggregation was found to be consistent with its enhanced platelet binding capacity. FIG. 17 shows that this mutant exhibits more effective inhibition of ristocetin-induced, vWF-dependent platelet aggregation in washed platelets, as compared to Y1 scFv (IC₅₀ of 0.2 μM and 0.8 μM respectively).

Taken together, these experiments demonstrate that an arginine residue at position 2 in the Y1-CDR3H may be relevant for binding to platelet GPIbα. Furthermore, such binding may involve electrostatic interaction since addition of an additional arginine residue at position 3 augmented both the platelet binding capacity and the anti-aggregation function.

### Example 5

The biological activity of A3R and of Y1 scFvs was further assessed in a Cone and Plate(let) Analyzer (CPA) assay, which is a new method for clinically evaluating whole blood platelet adhesion and aggregation on a polystyrene surface under high shear rates, thus mimicking physiological conditions (Varon et al., (1997) Thromb. Res. 85(4): 283-294; Shenkman et al., (2000) Thromb. Res. 99(4): 353-361).

These experiments were performed as follows. Blood samples (0.2 ml) were placed on non-tissue culture four-well polystyrene plates (unc, Rockilde, Denmark) and subjected to flow for 1 minutes at shear of 1300 s⁻¹, using a rotating Teflon cone, which was specifically designated for this system. The cone had a diameter of 14 mm with an angle of 2.45°, which induced a constant fluid shear stress over the entire plate surface. Wells were then thoroughly washed with PBS, stained with May-Grunwald stain and analyzed with an inverted light microscope (Olympus, Tokyo, Japan) connected to an image analysis system (Galai, Migdal Haemek, Israel).

The CPA assay was performed to evaluate the effect 1 μM (25 g/ml) and 21M (50 μg/ml) of scFv antibodies on platelet adhesion in CPA assay. As shown in FIG. 18, while the A3R mutant demonstrated a decrease in surface coverage from 13% in the control to 7% and 3% at concentration of 1 μM and 2 μM respectively. In comparison, Y1-scFv had no effect on surface coverage and was similar as in the control in both concentrations. These results indicated that A3R mutant inhibits platelet adhesion to polystyrene surface under flow conditions by the CPA via its binding to the GPIb receptor on platelets.

The average size (AS) of both antibodies was slightly decreased (data not shown). These results indicated that A3R scFv may inhibit platelet adhesion to polystyrene surface under flow conditions via its binding to the GPIb receptor on platelets.

On the basis of the observations that A3R binds sulfated GPIb and prevents interaction between GPIb and vWF which occurs exclusively under high shear stress conditions, as occurs in small arteries or as created by arterial stenosis, this antibody may be of therapeutic use for preventing and/or treating atherosclerotic disease.

### Example 6

The present example describes a comparison of the binding characteristics of S15, A3R, and Y1 to healthy platelet rich plasma (PRP).

FACS analysis was performed on PRP samples prepared from two healthy donors to analyze the binding of Y1, A3R, and S15 at different concentrations to platelets, the results of which are depicted in Table 6.

TABLE 6

| | | | |
|---|---|---|---|
| 0.1 μg | 0.5 μg | 1 μg | 2 μg |

PRP Sample #1

| | | | | |
|---|---|---|---|---|
| Y1 (P03) | Neg | Neg | Negative | 18 |
| A3R | Neg | Neg | 126 | |
| S15 | Neg | Neg | 30 | 120 |

PRP Sample #2

| | | | | |
|---|---|---|---|---|
| Y1 (P03) | Neg | Neg | Neg | 13 |
| A3R | Neg | Neg | 96 | |
| S15 | Neg | Neg | 23 | 68 |

*“Neg” stands for negative and is defined as when Geo Mean is below 10.

The results of Table 6 indicate that at concentrations of 1 μg, A3R binds to platelets with a stronger affinity that S15, which binds to platelets with a stronger affinity than Y1. At 1 μg, the average geo mean of both PRP samples was 116 for A3R, 26 for S15, and negative for Y1.

Selected scFv (0.5 μg per experiment) were analyzed by FACS to evaluate binding to PRP (Platelet Rich Plasma) expressing GPIb. As shown in Table 7, D1 and D3 exhibited significant binding to PRP, with D1 binding to a relatively greater extent. The level of D1 binding to PRP was similar to that exhibited by scFv S11 (LRYPFF) (SEQ ID NO:31), the selection of which is described in Example 1 of U.S. Ser. No. 10/611,238.

TABLE 7

| Clone | Geo Mean |
|---|---|
| D1 | 33.0 |
| D3 | 19.6 |
| S11 | 36.2 |

### Example 7

The present example describes a comparison of the binding characteristics of S15, A3R, and Y1 to healthy whole blood cells containing granulocytes (G), lymphocytes (L), and monocytes (M).

FACS analysis was performed on human whole blood samples from two healthy donors to analyze the binding of Y1, A3R, and S15 at different concentrations to whole blood cells, the results of which are depicted in Table 8.

TABLE 8

| | 0.1 μg | | | 0.5 μg | | | 1 μg | | | 2 μg | | |
|---|---|---|---|---|---|---|---|---|---|---|---|---|
| Antibody | L | G | M | L | G | M | L | G | M | L | G | M |

Whole Blood Sample #1

| | | | | | | | | | | | | |
|---|---|---|---|---|---|---|---|---|---|---|---|---|
| Y1 (P03) | neg | neg | neg | neg | neg | neg. | neg | neg | 30 | 20: | 30: | 57 |

| | | | | | | | | | | |
|---|---|---|---|---|---|---|---|---|---|---|
| A3R | neg | neg | neg | neg | 11 | 30 | 34 | 18 | 56 | ND |
| S15 | 36 | 26 | 56 | 81 | 100 | 192 | 120 | 150 | 290 | Saturated |

Whole Blood Sample #2

| | | | | | | | | | | | | |
|---|---|---|---|---|---|---|---|---|---|---|---|---|
| Y1 (P03) | neg | neg | neg | neg | neg, | neg. | neg | neg | 20 | 12 | 20 | 62 |

| | | | | | | | | | | |
|---|---|---|---|---|---|---|---|---|---|---|
| A3R | neg | neg | neg | neg | 14 | 30 | 25 | 53 | 120 | ND |
| S15 | 18 | 44 | 69 | 78 | 151 | 248 | 112 | 220 | 340 | Saturated |

The results of Table 8 indicate that the binding affinity of S15 to whole blood cells is higher than the binding affinity of A3R and Y1. At a 0.1 μg concentration, the average geo mean of S15 binding of both whole blood samples was medium to high, while the average geo mean of A3R and Y1 of both whole blood samples was negative or slightly positive at 0.5 μg.

scFvs D1, D3 and S11 (0.5 μg per experiment) were analyzed by FACS to evaluate binding to whole blood (gated on lymphocytes, monocytes and platelets). As shown in Table 9, D1 exhibited the greatest binding to all cell types, relative to the other two scFvs.

TABLE 9

| | Lymphocytes | Monocytes | Platelets |
|---|---|---|---|
| D1 | 23 | 90 | 36 |
| D3 | 3.8 | 21 | 6 |
| S11 | 12 | 38 | 17 |

### Example 8

This example describes the development of a non-human in vivo animal model system to further study the properties of the antibodies comprising the consensus sequence. An initial goal was to identify a non-human mammalian species in which one (or more) of the antibodies both (a) binds to platelets and (b) is capable of inhibiting platelet aggregation.

The ability of various scFv antibodies to bind to platelets isolated from different mammalian species was determined by FACS analysis and the relative binding results are summarized in Table 10.

TABLE 10

| Platelet Origin | Y1 | A3R | S15 | S11 | S1 | D1 |
|---|---|---|---|---|---|---|
| Human | + | ++++ | ++++ | +++++++ | +++++++ | +++++++ |
| Dog | +++ | ND | +++++++ | ++++++++ | ++++++++ | ++++++++ |
| Pig | + | ND | ++ | ND | ND | ND |
| Guinea pig | + | ++ | ++ | ++++ | ++++ | ++++ |
| Monkey | − | ND | − | ND | ND | ND |
| Mice | − | ND | − | ND | ND | ND |
| Rabbit | − | ND | − | ++++ | ++++ | ++++ |

ND = not determined.

Within each species tested, scFv Y1 showed the weakest relative binding among the antibodies comprising the consensus sequence. This observation supports the conclusion that the mutant (A3R) and library selected (S15, S11, S1 and D1) scFvs all have enhanced binding capacity for sulfated epitopes, as occur on platelet GPIb.

For example, scFvs A3R and S15 exhibit 5-9 fold greater (relative to Y1) binding capacity on human and guinea pig platelets, while binding capacity of S11, S1 and D1 to those species is about 25-fold greater than Y1.

With respect to the species array of non-human platelets bound by the panel of scFvs, S11, S1 and D1 each bind platelets derived from dog, guinea pig and rabbit. S15 binds platelets from dog (highest affinity), guinea pig and pig, but not from rabbit, mice or monkey (baboon and cynomalogus monkey). Importantly, guinea pig is the single non-human species identified in which all the antibodies comprising the consensus sequence are capable of binding platelets.

### Effect on Platelet Aggregation

Although S1, S11 and D1 bind to guinea pig derived platelets at much higher affinity relative to S15, all of these scFvs exhibit similar capacity with respect to inhibition of platelet aggregation.

The inhibitory effect of scFv S15 on guinea pig platelet aggregation (IC₅₀ 80-160 ug/ml) was found to be about 8-10 times lower relative to its inhibitory effect on human derived platelets. This may be due to the relative lower binding of scFv S15 antibody to guinea pig derived-platelets.

The binding of IgG1 S15 and IgG Y1 antibodies to human and to dog derived-platelets was found to be similar. The two antibodies induced platelet aggregation in human and in dog derived-platelets at the same concentration.

Pharmacokinetics of scFv in Guinea Pig

In order to assess the pharmacokinetics of scFv antibody in guinea pig, 10 mg/kg of scFv A3R was intravenously administrated to guinea pigs as a single bolus. The level of the scFv A3R in the blood of guinea pig at different time points was determined using ELISA assay and the level of the platelet bound scFv was determined using FACS analysis.

Three male guinea pigs (weight ˜500 g) were anesthetized with intraperitoneal injection of ketamine (50 mg/kg) mixed with xylazine (20 mg/kg). scFv A3R was administered by a bolus intravenous injection at 10 mg/kg (˜5 mg/guinea pig). Blood samples were collected at time 0 (before antibody administration) and at 3, 10 20, 30, 60, 90, 120, 180, and 360 minutes after administration of the antibody. At each time point, 0.5 ml blood was drawn. Sodium citrate at 3.8% was used as anticoagulant. Plasma was obtained by centrifuging blood samples at 3000×g for 15 minutes and stored at −20° C. and PRP was obtained by centrifuging blood samples at 150×g for 10 minutes. Platelet bound-A3R scFv was tested in guinea pig PRP by FACS analysis using anti-scFv PE-labeled antibodies. Plasma scFv A3R antibody concentrations were be assayed by a specific ELISA.

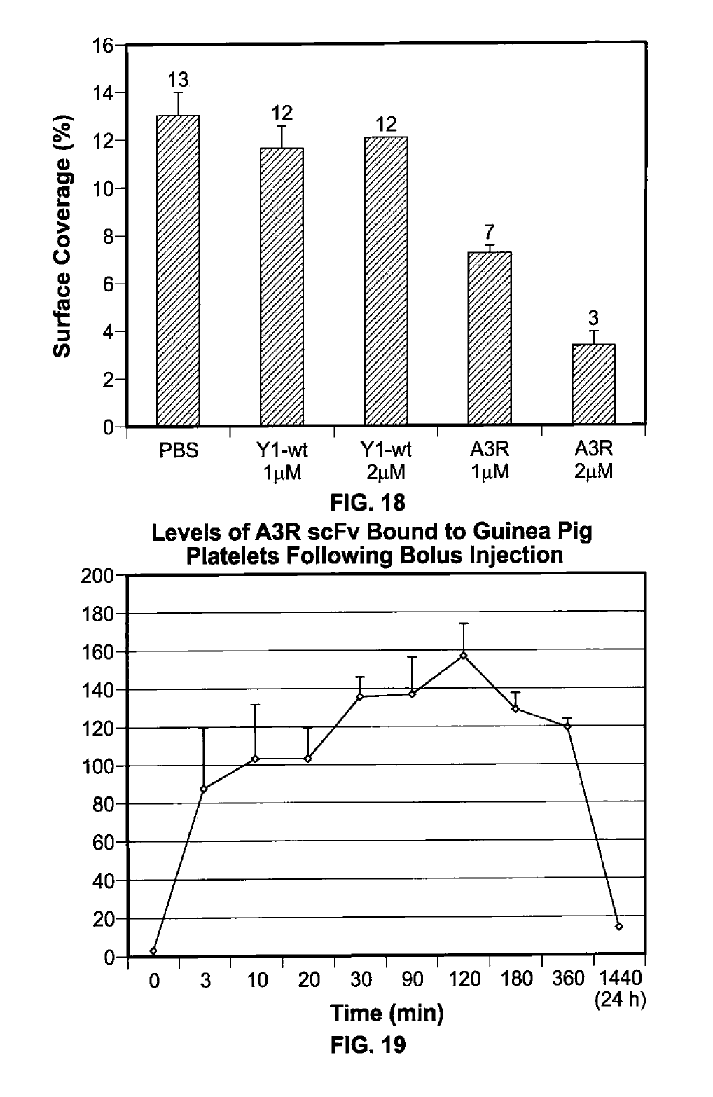

FACS analysis was used to evaluate the level of bound scFv-A3R antibody to guinea pig platelets at different time points post bolus administration. The results (FIG. 19) showed that at 10 minutes post-bolus administration of 10 mg/kg scFv-A3R, the level of bound scFv to platelets was 40-80% of the maximum level and reached the maximum level after 120 minutes The binding of scFv-A3R antibody to platelets remained high for 360 minutes (6 hour), and decreased by 90% by 24 hours post bolus administration.

Plasma level of A3R-scFv was measured by ELISA using plates coated with GPIb peptide having sulfated tyrosine at Tyr-276 (1 μM/well). Bound scFv was detected by addition of rabbit anti-VL (variable light) antibody, followed by anti-rabbit HRP (Sigma) and 3,3′,5,5′-tetramethyl-benzidine (TMB) (Sigma, St Louis, Mo.) substrate. The intensity of the color produced was read by an ELISA plate reader (Anthos, Salzburg) at O.D.450. The plasma concentration of the scFv in each sample was calculated from standard curves constructed by adding known amount of these antibodies to guinea pig plasma or to PBS containing 0.05% Tween 20 and 2% skim milk.

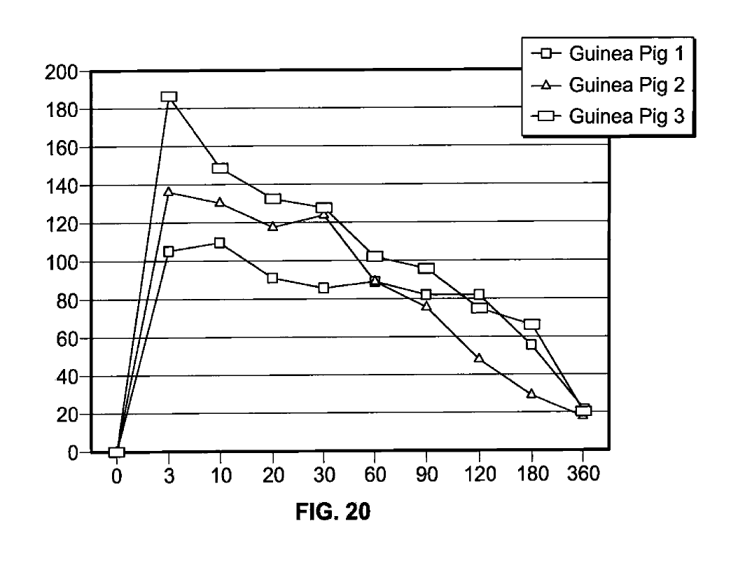

Results show that the plasma concentration of scFv A3R peaked 3 minutes after bolus injection and gradually declined over 6 hours (FIG. 20). The half-life of A3R in guinea pig was 139±9 minutes.

In summary, the above study provides a preliminary indication that A3R has a favorable pharmokinetic profile, based on the rapid achievement of saturation level on the target platelets and the relatively long half-life.

### Example 9

The BIAcore biosensor uses surface plasmon resonance detection and permits real-time kinetic analysis of two interacting species. This system was used to measure the binding kinetics of the scFvs Y1, A3R and S15 to glycocalicin, a polypeptide derived from platelet GPIb.

The binding affinities of antibodies for glycocalicin were determined using a BIAcore 3000 apparatus (BIAcore, Uppsala, Sweden), Glycocalicin (immobilized ligand) was covalently immobilized onto a CM5 chip (BIAcore) in 10 mM sodium acetate, pH 4.6, at a flow of 20 μl min⁻¹. All binding experiments were performed in RBS buffer, pH 7.4 (10 mM HEPES, 150 mM NaCl, 3.4 mM EDTA) including 0.005% (v/v) of the nonionic detergent, P20 (polyoxyethylene sorbitan). Binding affinities (Kᵢ) of the antibodies for glycocalicin were determined using BIAevaluation 3.1 software (BIAcore), from average Kₐ (association rate) and K_{d} (dissociation rate) kinetics.

A3R-scFv binds to platelet glycocalicin with higher affinity than the Y1-scFv (˜7 fold), as indicated by its more rapid association rate (Table 11). These results are in accordance with results obtained by FACS analysis on intact platelet. S15-scFv also binds to glycocalicin with higher affinity than the Y1-scFv but lower than A3R.

TABLE 11

| Immobilized | | Apparent | Apparent | K_{D} |
|---|---|---|---|---|
| Ligand | Analyte | Kₐ (μ⁻¹s⁻¹) | K_{d} (s⁻¹) | (M) |
| Glycocalicin | Y1-scFv | 2.7 × 10⁵ | 1.2 × 10⁻¹ | 4.5 × 10⁻⁷ |
| Glycocalicin | A3R-scFv | 1.99 × 10⁶ | 1.3 × 10⁻¹ | 6.7 × 10⁻⁸ |
| Glycocalicin | S15-scFv | 2.4 × 10⁶ | 4.3 × 10⁻¹ | 1.8 × 10⁻⁷ |

### Example 10

This example describes direct binding of antibodies comprising the consensus sequence to synthetic peptides based on those regions of PSGL-1, GPIb and CCR5 which undergo sulfation at tyrosine residues.

The synthetic peptides used were as follows.

| PSGL-1 (residues 42-58) derived | | |
|---|---|---|
| QATEYEYLDYDFLPETE | (non-sulfated) | SEQ ID NO:26 |
| QATEY^{S} EYLDYDFLPETE | (sulfated at 1^{st} tyrosine position) | SEQ ID NO:48 |
| QATEYEY^{S} LDYDFLPETE | (sulfated at 2^{nd} tyrosine position) | SEQ ID NO:49 |
| QATEYEYLDY^{S} DFLPETE | (sulfated at 3^{rd} tyrosine position) | SEQ ID NO:7 |
| GPIb (residues 268-285) derived | | |
| GDEGDTDLYDYYPEEDTE | (non-sulfated) | SEQ ID NO:43 |
| GDEGDTDLY^{S} DYYPEEDTE | (sulfated at 1^{st} tyrosine position) | SEQ ID NO:44 |
| GDEGDTDLYDYY^{S} PEEDTE | (sulfated at 3^{rd} tyrosine position) | SEQ ID NO:50 |
| CCR5 (residues 1-18) derived | | |
| MDYQVSSPIYDINYYTSE | (non-sulfated) | SEQ ID NO:51 |
| MDY^{s} QVSSPIYDINYYTSE | (sulfated at 1^{st} tyrosine position) | SEQ ID NO:52 |
| MDYQVSSPIY^{s} DINYYTSE | (sulfated at 2^{nd} tyrosine position) | SEQ ID NO:53 |
| MDYQVSSPIYDINY^{s} YTSE | (sulfated at 3^{rd} tyrosine position) | SEQ ID NO:54 |

The scFvs used were N06 (negative control), Y1 (PO3), S15, S11, S1, S9, s.c.3.1 and S11.

The various sulfated and non-sulfated peptides were adhered to NH CovaLink™ microtiter plates (Nunc, Denmark) following pre-treatment of plates with Sulfo-NHS (3.48 mg/ml) and EDC (3.07 mg/ml) for 30 minutes at room temperature, followed by one wash in water and three washes in PBS containing 0.05% Tween. Plates were blocked with PBS buffer containing 5% skim milk and 0.05% Tween using gentle shaking at room temperature for 1 hour. Synthetic peptides (100 μM per well) were added, followed by washing 5 times with PBS buffer containing 0.05% Tween and the plates were dried at room temperature. For binding, protein-A purified scFv antibodies (0.5 μg/well) were added to the plates for incubation for 1 hour at room temperature. For ELISA, rabbit anti-human V_{L} polyclonal antibody (anti-scFv) in blocking buffer was added to the plates for incubation at 25° C. for 60 minutes. Excess anti-scFv was removed by washing 5 times with PBS buffer containing 0.05% Tween. Goat anti-rabbit HRP-labeled antibody in blocking buffer was added for incubation for 1 hour at room temperature, and the excess was removed by washing 10 times. TMB Developer was added for 5 minutes and neutralized with of 0.5M H₂SO4 (100 μl/well). The color reaction was read by an ELISA plate reader at 450-nm wavelength. Each sample was assayed in duplicate and the average was calculated.

TABLE 12

| | PSGL-1 | | | | GPIb | | | CCR5 | | | |
|---|---|---|---|---|---|---|---|---|---|---|---|
| Sulfation | — | 1^{st} | 2^{nd} | 3^{rd} | — | 1^{st} | 3^{rd} | — | 1^{st} | 2^{nd} | 3^{rd} |
| N06 | 0.06 | 0.06 | 0.06 | 0.06 | 0.07 | 0.07 | 0.07 | 0.06 | 0.06 | 0.06 | 0.06 |
| P03 | 0.07 | 0.08 | 0.08 | 0.50 | 0.07 | 0.50 | 0.10 | 0.07 | 0.07 | 0.10 | 0.07 |
| S15 | 0.17 | .020 | 0.20 | 0.60 | 0.20 | 0.60 | 0.30 | 0.12 | 0.12 | 0.30 | 0.16 |
| S11 | 0.01 | 0.16 | 0.12 | 0.70 | 0.10 | 0.60 | 0.30 | 0.08 | 0.12 | 0.70 | 0.11 |
| S1 | .0.07 | 0.10 | 0.08 | 0.80 | 0.07 | 0.80 | 0.15 | 0.06 | 0.07 | 0.30 | 0.07 |
| S9 | 0.07 | 0.09 | 0.08 | 0.80 | 0.08 | 0.80 | 0.13 | 0.07 | 0.07 | 0.18 | 0.07 |
| s.c 3.1 | 0.07 | 0.09 | 0.09 | 0.80 | 0.08 | 0.80. | 0.13 | 0.07 | 0.08 | 0.25 | 0.08 |

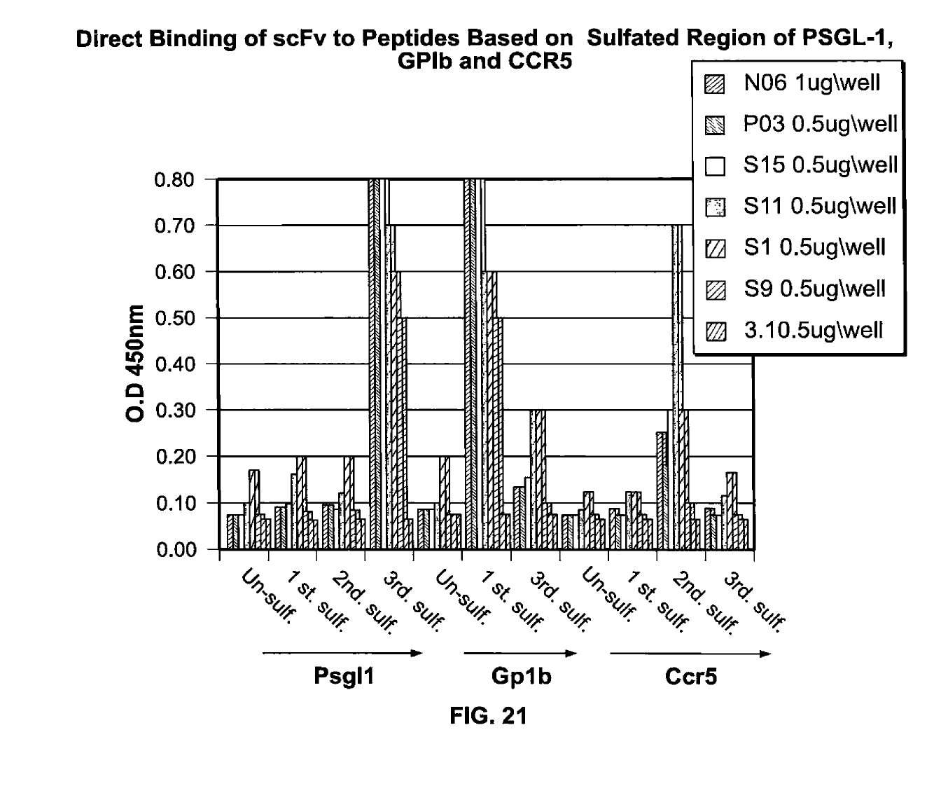

Table 12 and FIG. 21 indicate that all scFv antibodies tested comprising the consensus sequence bound significantly to PSGL-1-derived peptide sulfated at the third tyrosine position, but not to that sulfated at the first or second tyrosine position. Single chain antibodies S1, S9 and 3.1 showed the strongest binding to the PSGL-1-derived peptide sulfated at the third tyrosine position. These antibodies also bind to GPIb-derived peptide sulfated at the first tyrosine position, with scFvs S1, S9 and 3.1 showing the strongest binding and scFvs S15 and S11 showing intermediate binding to the GPIb derived peptide sulfated at the third tyrosine position. Significant binding of these antibodies to the CCR5-derived peptide sulfated at the second tyrosine position, but not to that sulfated at the first or third tyrosine position was also observed. Single chain antibody S11 showed the strongest binding to the CCR5 derived peptide sulfated at the second tyrosine position. No binding was observed with control antibody, scFv.

The results obtained with the CCR5 derived peptide sulfated at the second tyrosine position suggest that at least scFv S11 and possibly other antibodies comprising the consensus sequence may have potential as inhibitors of HIV infectivity in human cells.

### Example 11

Studies were carried out (in the laboratory of Dr. Tatjana Dragic, Albert Einstein College of Medicine, Yeshiva University) to evaluate the interaction of antibodies S11 and S15 with the HIV-1 coreceptors CCR5 and CXCR4.

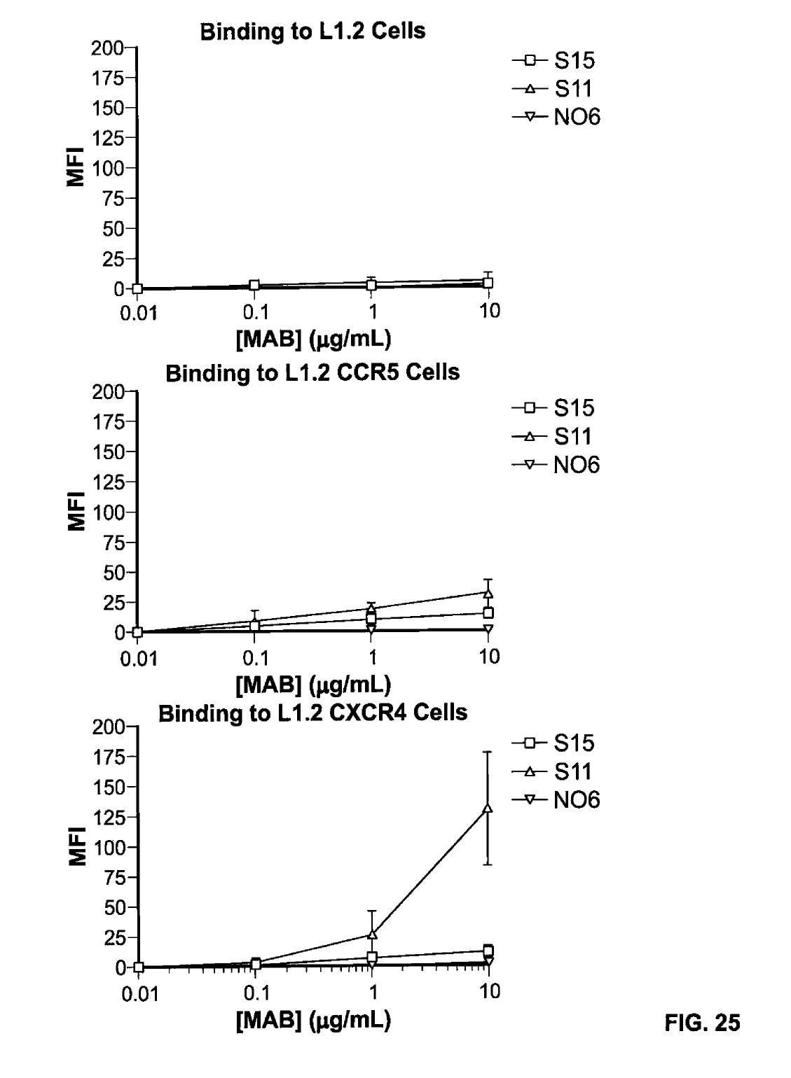

Binding of S1 (scFv) and S15 (IgG) to murine L1.2 hybridoma cells transfected to express either human CCR5 or human CXCR4 was analyzed by flow cytometry (for methods see Carnec et al. (2005) J. Virol. 79(3): 1930-1933). Detection of antibody binding was carried out using the phycoerythrin-labeled polyclonal anti-scFv antibody described in Example 4. The results, shown min FIG. 25, indicate that neither of the antibodies cross-react with murine CXCR4 or with murine PSGL-1, as evidenced by minimal binding to parental L1.2 cells. S11 binds with high affinity to cells expressing human CXCR4, and with low to moderate affinity to cells expressing human CCR5. S15 binds both coreceptors with low affinity, while the negative control (N06) exhibits negligible binding.

Next, binding to human cell lines (HeLa, endothelial cell line; CEMx174, T-B hybrid cell line; Jurkat, T-cell line; PM1, T-cell line; THP-1, monocytic cell line) that naturally express CXCR4 was examined (Carnec et al. (2005) J. Virol. 79(3):1930-1933). S11 and S15 bind with moderate to high affinity to each of the cell lines (with the exception of HeLa), and S11 binds with relatively greater affinity (FIG. 26).

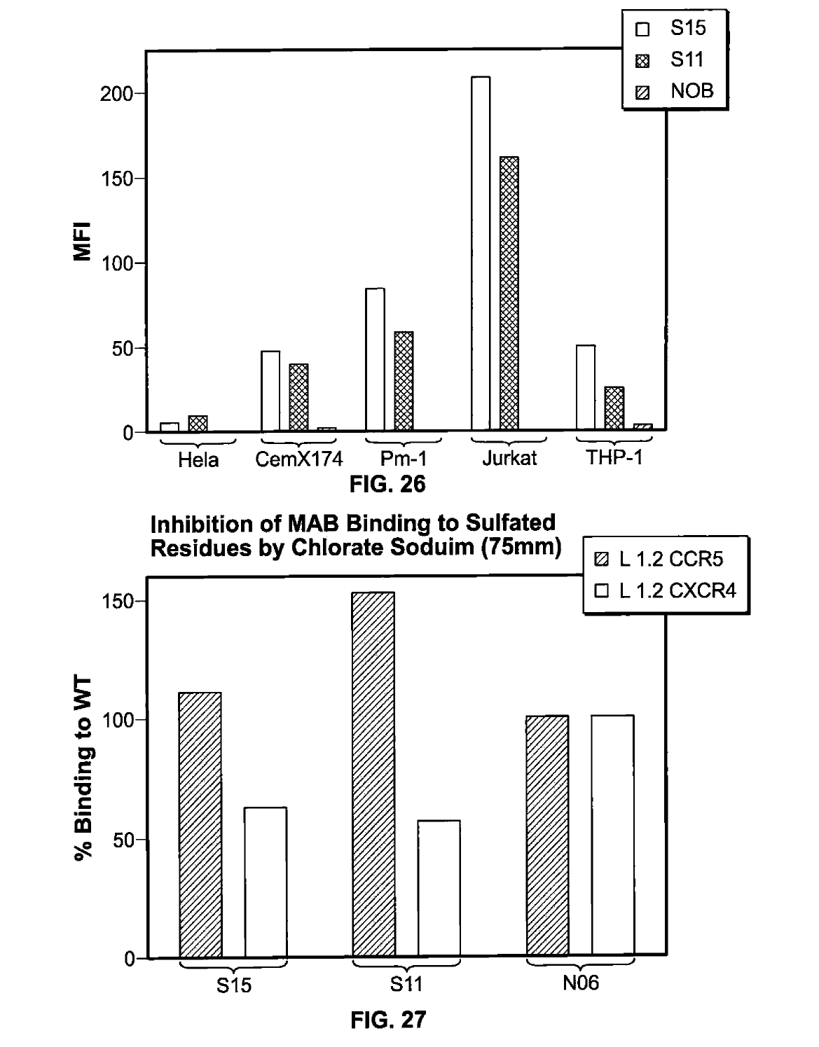

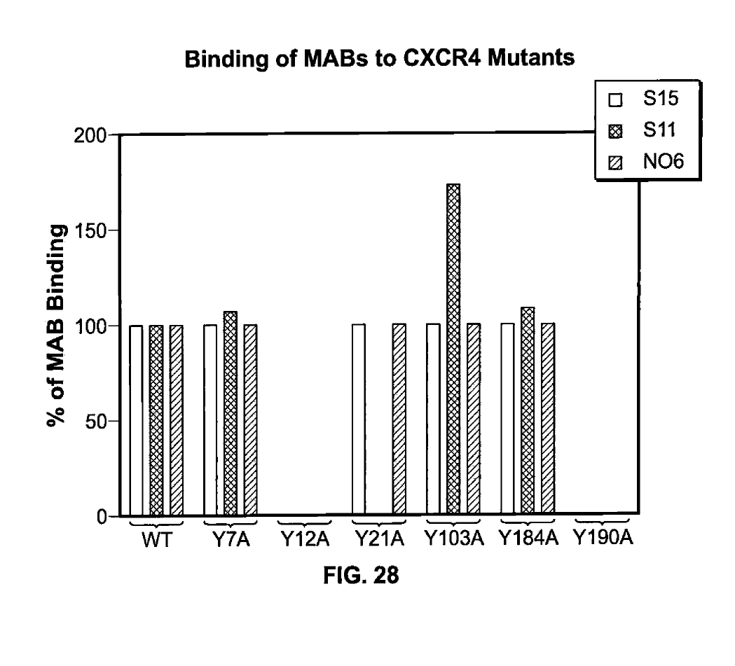

To evaluate the role of tyrosine sulfation on S11 and S15 binding, the L1.2 cells described above were grown in sulfate-free medium in the presence of 75 mM sodium chlorate to inhibit sulfation (Baeuerle et al. (1986) Biochem Biophys Res Commun 141 (2):870-877) and then examined for antibody binding by flow cytometry. As shown in FIG. 27, binding of S11 to L1.2 cells expressing CXCR4 was reduced by 50%, compared to control cells grown in complete medium lacking sodium chlorate. Similarly, binding of S15 to the same cells was reduced by about 40%. These results imply that sulfation is important for S11 and S115 binding to their epitopes on CXCR4.

Epitope mapping studies were then carried out with a panel of tyrosine-to-alanine point mutants of CXCR4, as described in Carnec et al (2005). Binding of S11 to CXCR4 was abolished when Tyr-21 was mutated to alanine, while mutation at Tyr-7, Tyr-103 and Tyr-184 had no effect on S11 binding. This suggests that Tyr-21, located in the N-terminal region of CXCR4, is a critical residue in the S11 epitope, whereas the other tyrosine residues examined, are not essential.

### Example 12

Studies were carried out to demonstrate that an IgG antibody comprising the consensus sequence of the invention is capable of mediating antibody dependent cell cytotoxicity (ADCC) of target cells, particularly B-CLL cells derived from patient samples. In addition, hyper cross-linking of S15-IgG with secondary anti-human Fc antibodies demonstrated that an apoptotic mechanism also contributes to cell killing.

Antibody-Dependent Cell Cytotoxicity (ADCC) experiments were as follows. Mononuclear effector and target cells were separated on FICOLL® and the target cells were then labeled with PKH26, which stably incorporates a fluorescent dye within the lipid regions of the cell membranes. Cells were then washed and incubated with effector cells at various Effector:Target (E:T) ratios, in the absence or in the presence of different concentrations of S15 IgG or control antibodies for 24 hours. Dead cells were stained by TOPRO® (Molecular Probes, Inc., Eugene, Oreg.) and analyzed by FACS on gated target cells.

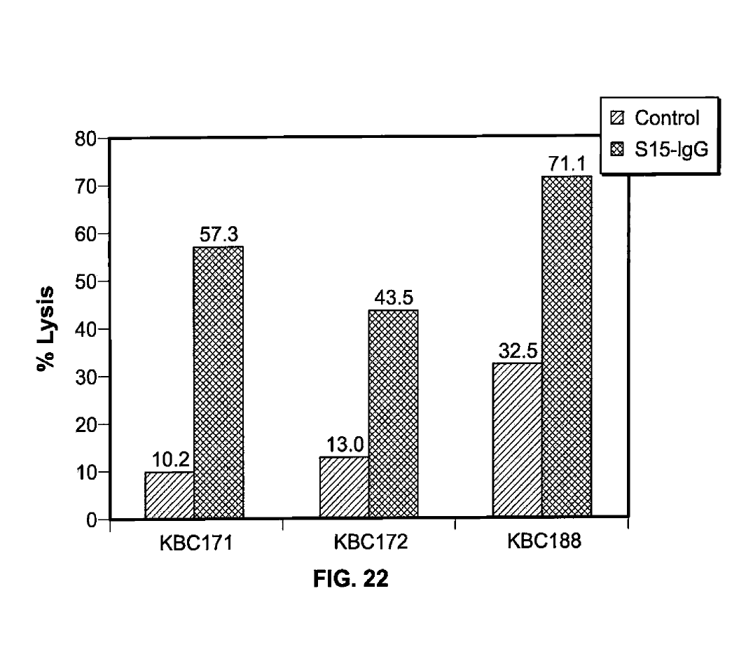

Mononuclear effector cells from healthy donors and B-CLL target cells from three patients were co-incubated at various E:T ratios in the presence and absence of S15-IgG for 24 hours, followed by FACS analysis. FIG. 22B shows that S15 IgG mediated effector cell cytotoxicity in all three samples, with 30-500% ADCC as compared to control.

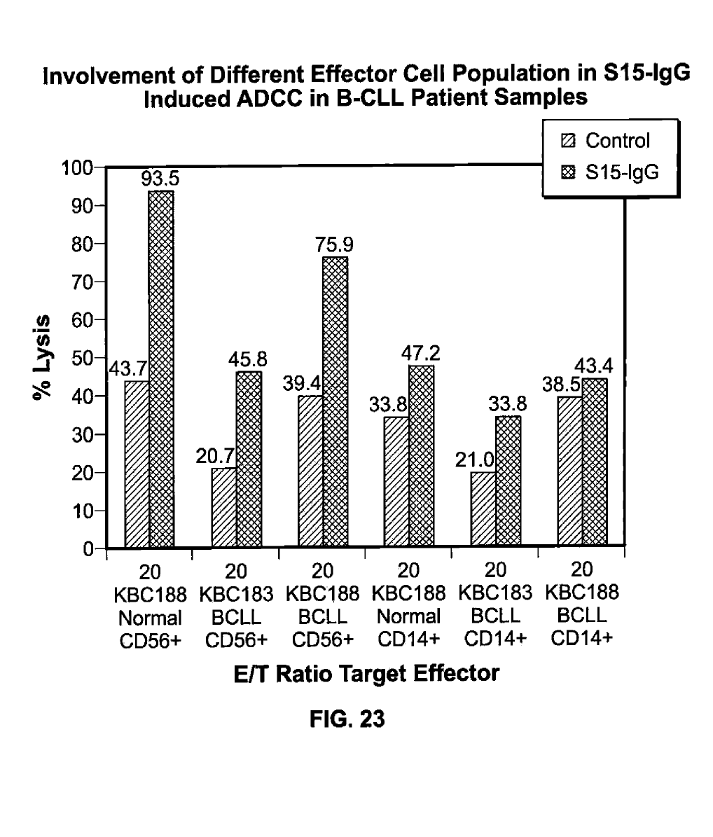

S15-IgG ADCC is mediated by natural killer and monocytic cells. Effector cells were analyzed for their capability of effecting S15 IgG-mediated ADCC of B-CLL cells. Natural killer (CD56+) and monocytic (CD14+) cells from both normal donors and B-CLL patients were isolated using commercially available magnetic beads. FIG. 23 shows that NK cells from a normal donor and B-CLL patients were capable of effecting ADCC, resulting in about 50% and 35% killing respectively. Monocytes from both a normal donor and from B-CLL patients were also capable of effecting ADCC (about 5-13%). However, CD56+NK cells constituted the more significant effector cell population for S15 IgG-mediated ADCC of B-CLL cells.

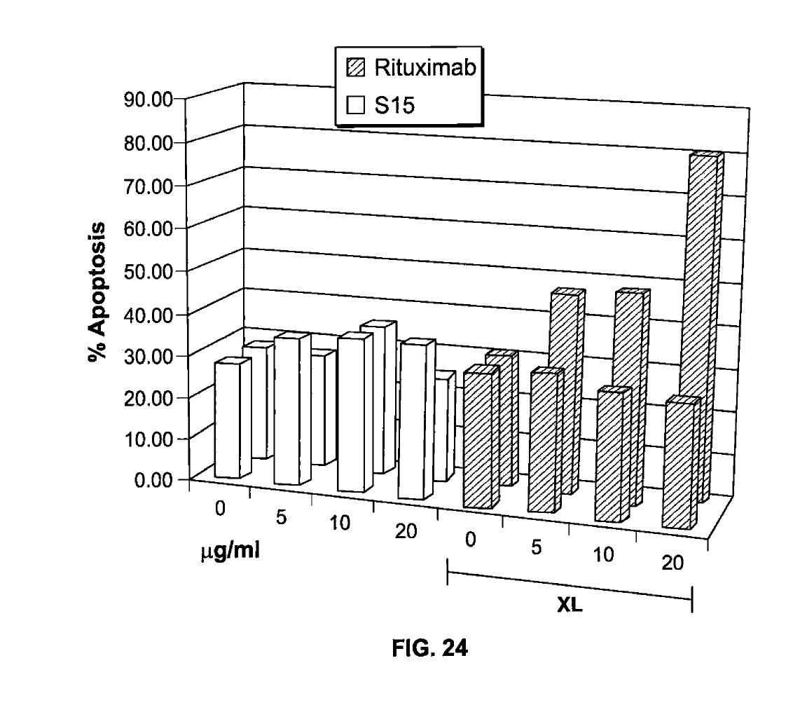

Apoptosis experiments were as follows. Mononuclear cells from B-CLL patients were separated on FICOLL® and the cells were incubated in the presence or in the absence of S15-IgG or control antibodies for 10 minutes at 37° C. Anti-human Fc antibodies were then added and incubated for 4-24 hours at 37° C. Diseased cells (CD19+,CD5+) were then stained with apoptotic markers Annexin and TOPRO® and analyzed by FACS. S15-IgG induced apoptosis. FACS analysis showed that mononuclear cells (CD19+, CD5+) from B-CLL patients incubated in the presence of S15-IgG exhibited about 10% apoptosis within 5 hours (FIG. 24). Addition of secondary antibodies, which cross-link the S15-IgG, elicited an additional 50% of apoptosis within 5 hours (FIG. 24).

This constitutes strong evidence that cross-linking of an antibody directed to a sulfated epitope on PSGL-1 triggers signals for apoptosis of primary B-CLL cells. This implies that PSGL1 can be a target for inducing apoptosis in B-CLL patients in vivo, wherein the cross-linking effect may be mediated by Fc receptor bearing cells e.g. monocytes, CD56⁺ NK-cells and γδ⁺ T-cells.

In contrast to S15-IgG, the humanized antibody Rituximab, which is used extensively for treatment of various lymphoid malignancies including B-CLL, showed no augmentation of apoptosis upon cross-linking (FIG. 25).

The apoptotic and cross-linking effects described above for S15-IgG may be inhibited using the murine antibody KPL1 directed against PSGL-1, which on its own does not induce apoptosis (data not shown). This provides confirmation that the apoptotic signal is mediated via an epitope on PSGL-1.

Furthermore, FACS analysis showed that S15 IgG binds to all B-CLL samples tested, in contrast to its marginal binding to normal B-cells. Taken together, the results suggest that S15-IgG is a promising candidate as a therapeutic agent in the treatment of B-CLL, as its cytotoxic and apoptotic effects appear to be mediated via specific recognition of a PSGL-1 sulfated epitope expressed on these diseased cells.

### Example 13

### Screening of Inorganic Compound Library

Synthetic sulfated peptide (sulfated on a given specific tyrosine residue within the known amino acid sequence of the peptide) derived from a specific receptor (protein) can be prepared with a biotin tag (biotinylated) coupled to the synthetic peptide via a short linker such as caproic acid. Control peptides using the same synthetic peptide can be prepared without sulfation and without the biotin tag (“B”). In addition, synthetic sulfated peptides derived from other, non-related proteins can be prepared without having the biotin tag (“C”) as additional controls.

The biotinylated peptide above (“A”) can be coupled to strepavidin-coated magnetic beads and excess unbound biotinylated peptide then washed away. The biotin-stretavidin peptide conjugate (“D”) can be screened against a small chemical entity library in the presence of large excess of non-sulfated control peptide (“B”) under physiological conditions (37° C., pH 7.0-7.4, salts concentration, conductivity etc.) for molecules that bind to “A”. The coupled-magnetic beads are then washed twice with buffer, each time centrifuged to remove excess unbound molecules. Molecules bound to the magnetic beads (“E”) can be eluted, chemically identified and prepared in larger quantity for further screening.

Confirmation of binding to biotinylated sulfated peptides by the selected chemical compounds (“E”) can be carried out by a further screening process. This process includes either competition with unrelated sulfated peptides that are biotinylated (process 1) or competition with an antibody or fragments thereof (e.g. scFv) that bind specifically to the biotinylated peptide, “A” (process 2).

Re-screening by competition with unrelated biotinylated sulfated peptides (Process 1). In order to ensure that the compounds bind specifically to “A”, a second round of screening can be carried out. Biotin-streptavidin peptide conjugate (“D”) can be re-screened with the selected compounds “E” in the presence of large excess of unrelated biotinylated sulfated peptides, “C”. The tube is then centrifuged, the biotin-stretavidin peptide conjugate coupled magnetic beads washed twice with buffer and centrifuged each time to remove excess unbound molecules. Compounds that bound to the magnetic beads can be eluted for chemical identification. Larger quantities of the chemical compound can be prepared for further studies, such as validation of selective binding to “A”, and efficacy testing in vitro and in vivo.

Re-screening by competition with specific scFv anti-sulfated antibody (Process 2) was as follows. Compounds with preferred binding affinity to “A” can be re-screened by competing the binding of biotin-streptavidin peptide conjugate (“D”) to each of the selected compounds “E” in the presence of large excess of specific scFv antibody that specifically recognizes and binds to “A”. Chemical compounds that are specifically inhibited from binding to “A” by the scFv antibody can be prepared for further studies, such as validation of selective binding to “A”, and efficacy testing in vitro and in vivo.

The foregoing description and examples have been set forth merely to illustrate the invention and are not intended to be limiting. Since modifications of the disclosed embodiments incorporating the spirit and substance of the invention may occur to persons skilled in the art, the invention should be construed to include everything within the scope of the appended claims and equivalents thereof.

We claim:

1. An antibody or fragment thereof comprising a consensus sequence:
X₁-X₂-X₃-Pro-X₅-X₆
wherein X₁ and X₆ are hydrophobic amino acids and X₂ X₃ and X₅ are any amino acid.

2. The antibody or fragment thereof of claim 1, wherein the antibody or fragment thereof comprises a first heavy chain complementarity determining region (CDR) selected from the group consisting of SEQ ID NO:9, 10, 13, 14, 28 and 31; a second heavy chain CDR comprising an amino acid sequence of SEQ ID NO:17 and a third heavy chain CDR comprising an amino acid sequence of SEQ ID NO:18.

3. The antibody or fragment thereof of claim 1, wherein the antibody or fragment thereof binds to one or more epitopes, sulfated at one or more positions present on a protein selected from the group consisting of: α-2 antiplasmin; aminopeptidase B; a CC chemokine receptor; a seven-transmembrane-segment (7TMS) receptor; coagulation factors V, VIII, and IX; fibrinogen gamma chain; heparin cofactor II; secretogranin I and II; vitronectin, amyoid precursor, α-2-antiplasmin; cholecystokinin; α-choriogonadotropin; complement C4; dermatan sulfateproteoglycan; fibronectin; and castrin.

4. The antibody or fragment thereof of claim 18, wherein the CC-chemokine receptor is selected from the group consisting of CCR2, CCR5, CCR3, CXCR3, CXCR4, CCR8, and CCR2b.

5. A pharmaceutical composition comprising the antibody or fragment thereof of claim 1 and a pharmaceutically acceptable carrier.

6. A method of treating an infection caused by HIV comprising administering to a patient in need thereof of pharmaceutical composition of claim 5.

7. The method of claim 6, wherein the administration prevents entry of HIV.
